(19) United States
(12) Patent Application Publication
(10) Pub. No.: US 2026/0034355 A1
(43) Pub. Date: Feb. 5, 2026

(54) METHODS OF IMPROVING MOTOR LEARNING

(71) Applicants: Diego ARIAS, Charleston, SC (US); Christopher BUNEO, Gilbert, AZ (US)

(72) Inventors: Diego ARIAS, Charleston, SC (US); Christopher BUNEO, Gilbert, AZ (US)

(73) Assignee: Arizona Board of Regents on Behalf of Arizona State University, Scottsdale, AZ (US)

(21) Appl. No.: 19/284,556

(22) Filed: Jul. 29, 2025

Related U.S. Application Data
(60) Provisional application No. 63/677,370, filed on Jul. 30, 2024.

(51) Int. Cl. A61N 1/36

(52) U.S. Cl. CPC A61N 1/36003; A61N 1/36025; A61N 1/36034

(57) ABSTRACT

The present disclosure relates a novel method of improving motor learning by stimulating the trigeminal nerves and/or the occipital nerves. In particular implementations, the method of improving motor learning in a subject comprising stimulating the trigeminal nerves and/or the occipital nerves of the subject with electrical stimulation of 50-150 Hz or 1-5 kHz. The electrical current is administered as a biphasic symmetric square waveform in 30 s intervals.

### RELATED APPLICATIONS

This application claims priority to and the benefit of U.S. provisional patent application 63/677,370, filed Jul. 30, 2024, titled “Methods of Improving Motor Learning,” the entirety of the disclosure of which is hereby incorporated by this reference.

### STATEMENT REGARDING FEDERALLY SPONSORED RESEARCH OR DEVELOPMENT

This invention was made with government support under N66001-17-2-4018 awarded by DARPA (Defense Advanced Research Projects Agency). The government has certain rights in the invention.

### TECHNICAL FIELD

This document relates to nerve stimulation methods for improving motor learning.

### BACKGROUND

A current thrust in neurorehabilitation research involves exogenous neuromodulation of peripheral nerves to enhance neuroplasticity and maximize recovery of function.

Pharmacological and non-pharmacological techniques have been used to modulate activity in the brain. Among the non-pharmacological methods, one approach is to stimulate the cortex directly to evoke a cortical response. Transcranial Direct Current Stimulation (tDCS) and Transcranial Magnetic Stimulation (TMS) are examples of these techniques. These methods try to depolarize brain areas in a top-down fashion using electric current and magnetic fields, respectively. Another approach is to activate the cortex indirectly via the stimulation of bottom-up pathways. Several studies have shown that it is possible to generate brain excitability by applying electrical stimulation to specific peripheral nerves such as the vagus (Cranial Nerve X), trigeminal (Cranial Nerve V), and occipital nerves.

Vagus Nerve Stimulation (VNS) is currently approved for stroke rehabilitation, setting a precedent for this bottom-up mechanism to modulate motor behavior. This makes sense, as the vagus nerve is both a sensory nerve as well as a motor nerve. However, it is unknown whether stimulation of the trigeminal and occipital nerves, which share anatomical projections, would produce similar results. Unlike the vagus nerve, the trigeminal nerves and occipital nerves are primarily sensory nerves. Only the mandibular nerve branch of the trigeminal nerve and the third occipital comprise motor fibers.

### SUMMARY

In one aspect, the present invention provides for a method of improving motor learning in a subject comprising delivering an electrical current to the subject's trigeminal nerves. In certain embodiments, the electrical current to the subject's trigeminal nerves is 2.0-4.5 mA. In certain embodiments, the electrical current delivered to the subject's trigeminal nerves has a frequency of 50-150 Hz or about 3 kHz. In certain embodiments, the electrical current is delivered as a biphasic symmetric square waveform in 30 s intervals. In certain embodiments, the electrical current is delivered a pulse width 250 μs and an interphase interval is 1 μs. In certain embodiments, the electrical current is delivered to the left and right supraorbital branches of the subject's trigeminal nerve. In certain embodiments, the subject's visuomotor adaptation is improved. In certain embodiments, the subject does not have a brain injury or has not been diagnosed with a brain disease or disorder. In certain embodiments, the subject is a healthy subject. In certain embodiments, the subject is recovering from a stroke.

In another aspect, the present invention provides for a method of improving motor learning in a subject comprising delivering an electrical current to the subject's occipital nerves. In certain embodiments, the electrical current to the subject's trigeminal nerves is 9.5-17 mA. In certain embodiments, the electrical current delivered to the subject's trigeminal nerves has a frequency of 1-5 Hz or about 3 kHz. In certain embodiments, the electrical current is delivered as a biphasic symmetric square waveform in 30 s intervals. In certain embodiments, the electrical current is delivered a pulse width 250 μs and an interphase interval is 1 μs. In certain embodiments, the electrical current is delivered to target the subject's C2/C3 dermatomes. In certain embodiments, the subject's visuomotor adaptation is improved. In certain embodiments, the subject does not have a brain injury or has not been diagnosed with a brain disease or disorder. In certain embodiments, the subject is a healthy subject. In certain embodiments, the subject is recovering from a stroke.

The foregoing and other aspects, features, and advantages will be apparent from the DESCRIPTION and DRAWINGS, and from the CLAIMS if any are included.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The features and advantages of the present disclosure, and the manner of attaining them, will become more apparent and the present disclosure will be better understood by reference to the description of the present disclosure taken in conjunction with the accompanying drawings.

FIG. 1 depicts, in accordance with certain embodiments, the semi-immersive 3D VR environment used during the experiments. Subjects were seated with their head positioned on a chinrest and their eyes aligned with the center of the mirror as their arm movements were captured by a motion tracking system.

FIG. 2A depicts, in accordance with certain embodiments, the location of targets relative to the starting position. FIG. 2B depicts, in accordance with certain embodiments, the 3D environment showing the hand position (green sphere), target (red sphere), cage, and performance feedback given to participants (top).

FIGS. 3A-3C depict, in accordance with certain embodiments, characteristic trajectories during baseline (FIG. 3A), rotation (FIG. 3B), and washout block (FIG. 3C). FIG. 3D depicts, in accordance with certain embodiments, the expected directional error profile during a visuomotor rotation task.

FIG. 4 depicts, in accordance with certain embodiments, the stimulation parameters of the disclosed nerve stimulation methods.

FIG. 5A depicts, in accordance with certain embodiments, parameters obtained from velocity profile. FIG. 5B depicts, in accordance with certain embodiments, illustration of directional error.

FIG. 6A depicts, in accordance with certain embodiments, directional error (DE) during the rotation block fitted to a double exponential model. FIG. 6B depicts, in accordance with certain embodiments, DE during the washout block fitted to a single exponential model.

FIG. 7 depicts, in accordance with certain embodiments, experimental design for offline TNS experiments.

FIG. 8 depicts, in accordance with certain embodiments, mean reaction time (±SEM) for the 120 Hz (red), 60 Hz (black) and sham (blue) groups across experiment 1A.

FIG. 9 depicts, in accordance with certain embodiments, mean peak velocity (±SEM) for the 120 Hz (red), 60 Hz (black) and sham (blue) groups across experiment 1A.

FIG. 10A depicts, in accordance with certain embodiments, learning curves for the 120 Hz (red), 60 Hz (black) and sham (blue). FIGS. 10B and 10C respectively depict, in accordance with certain embodiments, fast and slow rates obtained from a double exponential model fitted to the average DE data for each group.

FIG. 11A depicts, in accordance with certain embodiments, DE during washout block fitted to a single-exponential model. FIG. 11B depicts, in accordance with certain embodiments, forgetting rates were not significantly different between groups.

FIG. 12 depicts, in accordance with certain embodiments, learning curve for the sham group was split into participants who experienced the stimulation (Sham-felt, dashed blue line) and who did not (Sham-no, dotted blue line).

FIG. 13 depicts, in accordance with certain embodiments, side effects reported during experiment 1A. Specifically, subjects were consulted for blurry vision, headache, dizziness, and skin itching at the electrode site.

FIG. 14 depicts, in accordance with certain embodiments, mean reaction time (±SEM) for the 3 kHz (maroon) and sham (gold) groups across experiment 1B.

FIG. 15 depicts, in accordance with certain embodiments, mean peak velocities (±SEM) for the 3 kHz (maroon) and sham (gold) groups across experiment 1B.

FIG. 16A depicts, in accordance with certain embodiments, learning curves for the 3 kHz (maroon) and sham (blue). FIGS. 16B and 16C respectively depict, in accordance with certain embodiments, fast and slow rates obtained from a double exponential model fitted to the average DE data for each group.

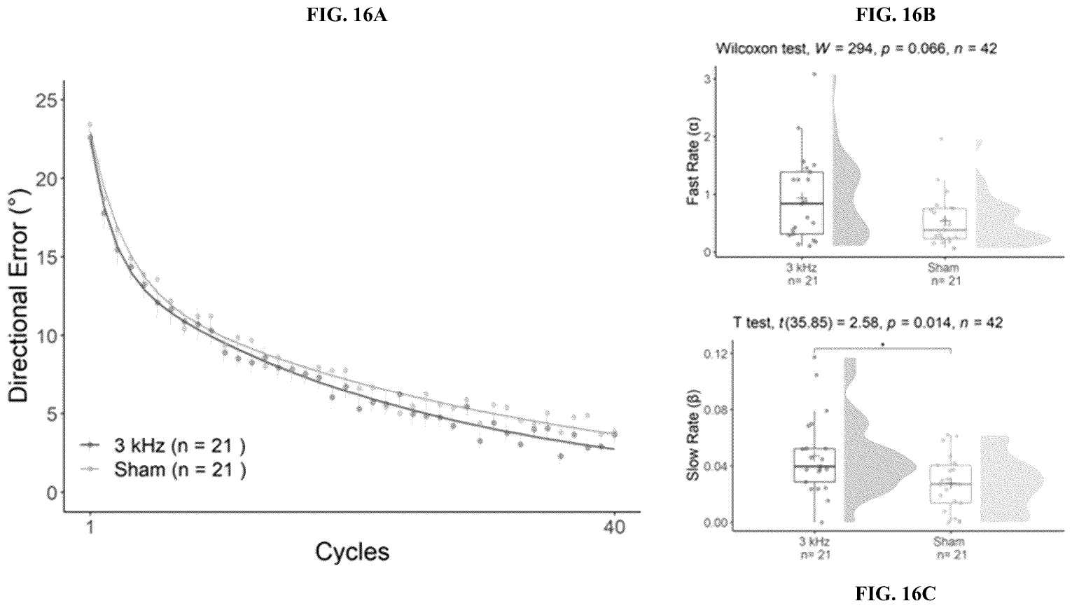

FIGS. 17A-17D depict, in accordance with certain embodiments, bootstrap distributions (FIGS. 17A and 17B) and results of permutation tests (FIGS. 17C and 17D) performed on the fast and slow rate coefficients for experiment 1B.

FIG. 18A depicts, in accordance with certain embodiments, directional error during the washout block. FIG. 18B depicts, in accordance with certain embodiments, forgetting rate.

FIG. 19 depicts, in accordance with certain embodiments, learning curve for the sham group in experiment 1B split into participants who experience the stimulation (Sham-felt) and who did not (Sham-no).

FIG. 20 depicts, in accordance with certain embodiments, side effects reported during experiment 1B. Subjects were consulted for blurry vision, dizziness, headache, and skin itching at the electrode position during or after the experiments.

FIG. 21 depicts the study design for experiment 2. TNS was applied online.

FIG. 22 depicts, in accordance with certain embodiments, mean reaction time (±SEM) for the 120 Hz (red), 60 Hz (black) and sham (blue) groups across experiment 2.

FIG. 23 depicts, in accordance with certain embodiments, mean peak velocity (±SEM) for the 120 Hz (red), 60 Hz (black) and sham (blue) groups across experiment 2.

FIG. 24A-24C depict, in accordance with certain embodiments, learning curves (FIG. 24A), fast rate (FIG. 24B) and slow rates (FIG. 24C) analysis for the online study.

FIG. 25A depicts, in accordance with certain embodiments, single exponential model fitted to the DE washout data. FIG. 25B depicts, in accordance with certain embodiments, rate analysis.

FIG. 26 depicts, in accordance with certain embodiments, post-study responses sorted by group when participants were consulted for headaches, blurry vision, dizziness and skin itching.

FIG. 27 depicts, in accordance with certain embodiments, sham directional error was split into subjects who felt (Sham-felt) the stimulation and who did not (Sham-no). Learning curves for the active groups and the subgroups of the sham are displayed (Sham-no: blue-dotted line, Sham-felt: dashed-line).

FIG. 28A depicts, in accordance with certain embodiments, learning curves fitted to the DE for the 120 Hz group applied online (maroon bold line) and offline (red bold line). FIGS. 28B and 28C respectively depict, in accordance with certain embodiments, fast and slow rates comparison between 120 Hz applied offline and online.

FIG. 29A depicts, in accordance with certain embodiments, learning curves fitted to the DE for the 60 Hz group applied online (gray bold line) and offline (black bold line). FIGS. 29B and 29C respectively depict, in accordance with certain embodiments, fast and slow rates comparison between 60 Hz applied offline and online.

FIG. 30 depicts the experimental design for the ONS/TNS study.

FIG. 31 depicts the schematic diagram of TNS/ONS setup.

FIGS. 32A-32D depict, in accordance with certain embodiments, the stimulation protocol for TNS+ONS (FIG. 32A), TNS (FIG. 32B), ONS (FIG. 32C), and sham groups (FIG. 32D). Stimulation applied on the forehead and the back of the head was delivered using two DS8R stimulators (Stim 1 and 2), one for each location.

FIG. 33 depicts, in accordance with certain embodiments, current tolerated during ONS delivered independently (ONS) and concurrently with TNS (TNS+ONS) (left) and current tolerated during TNS delivered independently (TNS) and concurrently with ONS (TNS+ONS) (right). No significant differences were found between the groups.

FIG. 34 depicts, in accordance with certain embodiments, mean reaction time (±SEM) for the TNS (red), ONS (black), TNS+ONS (maroon), and sham (blue) groups across experiment 3.

FIG. 35 depicts, in accordance with certain embodiments, mean peak velocity (±SEM) for the TNS (red), ONS (black), TNS+ONS (maroon), and sham (blue) groups across experiment 3.

FIG. 36A depicts, in accordance with certain embodiments, learning curves. FIGS. 36B and 36C respectively depict, in accordance with certain embodiments, fast and slow rates comparison.

FIGS. 37A and 37B respectively depict, in accordance with certain embodiments, the forgetting curve and the forgetting rates comparison.

FIG. 38 depicts, in accordance with certain embodiments, post-study responses sorted by group when participants were consulted for blurry vision, dizziness, headache and skin itching at the electrodes position during or after the experiments.

FIG. 39A depicts, in accordance with certain embodiments, a sham group split into participants who perceived the stimulation and improvement in motor performance (Sh-felt-imprv), who perceived the stimulation but no motor improvements (Sh-felt-no-imprv), and who did not perceive the stimulation at all (Sh-no). FIG. 39B depicts, in accordance with certain embodiments, sham learning curve after the removal of the subgroup Sh-felt-imprv compared to active groups.

FIG. 40 depicts, in accordance with certain embodiments, learning curves for all the passive sham interventions in experiments 1A, 1B, and 2 (blue line) and the active sham approach (red line).

### DETAILED DESCRIPTION

Detailed aspects and applications of the disclosure are described below in the following drawings and detailed description of the technology. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth in order to provide a thorough understanding of the various aspects of the disclosure. It will be understood, however, by those skilled in the relevant arts, that embodiments of the technology disclosed herein may be practiced without these specific details. It should be noted that there are many different and alternative configurations, devices and technologies to which the disclosed technologies may be applied. The full scope of the technology disclosed herein is not limited to the examples that are described below.

The singular forms “a,” “an,” and “the” include plural referents unless the context clearly dictates otherwise. Thus, for example, reference to “a step” includes reference to one or more of such steps.

The word “exemplary,” “example,” or various forms thereof are used herein to mean serving as an example, instance, or illustration. Any aspect or design described herein as “exemplary” or as an “example” is not necessarily to be construed as preferred or advantageous over other aspects or designs. Furthermore, examples are provided solely for purposes of clarity and understanding and are not meant to limit or restrict the disclosed subject matter or relevant portions of this disclosure in any manner. It is to be appreciated that a myriad of additional or alternate examples of varying scope could have been presented but have been omitted for purposes of brevity.

When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

Throughout the description and claims of this specification, the words “comprise” and “contain” and variations of the words, for example “comprising” and “comprises”, mean “including but not limited to”, and are not intended to (and do not) exclude other components.

As required, detailed embodiments of the present disclosure are included herein. It is to be understood that the disclosed embodiments are merely exemplary of the invention that may be embodied in various forms. Therefore, specific structural and functional details disclosed herein are not to be interpreted as limits, but merely as a basis for teaching one skilled in the art to employ the present invention. The specific examples below will enable the disclosure to be better understood. However, they are given merely by way of guidance and do not imply any limitation.

The present disclosure may be understood more readily by reference to the following detailed description taken in connection with the accompanying figures and examples, which form a part of this disclosure. It is to be understood that this disclosure is not limited to the specific materials, devices, methods, applications, conditions, or parameters described and/or shown herein, and that the terminology used herein is for the purpose of describing particular embodiments by way of example only and is not intended to be limiting of the claimed inventions. The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

Disclosed herein are methods of improving motor learning, for example visuomotor learning, through stimulation of the trigeminal nerves or the occipital nerves. Thus, the methods improve human performance, for example as measured by reaction time or peak velocity of movement. In one aspect, a method of reducing reaction times of a subject is disclosed. The method comprises delivering an electrical current to the subject's trigeminal nerves or occipital nerves.

The disclosed methods are usable to improve motor learning in patients with disease or disorders related to the brain (for example, stroke) or in patients recovering from brain injury (for example, traumatic brain injury). The improvement in human performance is also observable when the disclosed methods of stimulation the trigeminal nerve or the occipital nerve are administered to subjects without any diseases or disorders of the brain or injury to the brain. Thus, in one aspect, the subject being administered nerve stimulation does not have a brain injury or has not been diagnosed with a brain disease or disorder, for example, a healthy subject. In another aspect, the subject being administered nerve stimulation is recovering from a brain injury or has been diagnosed with a brain disease or disorder. In a particular implementation, the subject is recovering from a stroke. The beneficial effects on motor learning from stimulation of the trigeminal nerves or the occipital nerves depends on the frequency of stimulation and timing relative to the execution of a task.

In yet another aspect, the subject being administered nerve stimulation is an older adult, for example a human over the age of 50 years, over the age of 60 years, over the age of 65 years, over the age of 70 years, over the age of 75 years, over the age of 80 years, or over the age of 85 years. In some implementation, the subject is a human between the ages of 18 years and 36 years, between the ages of 24 years and 45 years, between the ages of 35 years and 45 years, between the ages of 35 years and 50 years, between the ages of 35 years and 55 years, between the ages of 35 years and 60 years, between the ages of 35 years and 65 years, between the ages of 35 years and 70 years, between the ages of 35 years and 75 years, between the ages of 35 years and 80 years, between the ages of 35 years and 85 years, between the ages of 40 years and 60 years, between the ages of 40 years and 65 years, between the ages of 40 years and 70 years, between the ages of 40 years and 75 years, between the ages of 40 years and 80 years, between the ages of 40 years and 85 years, between the ages of 50 years and 85 years, between the ages of 55 years and 85 years, between the ages of 60 years and 85 years, between the ages of 65 years and 85 years, between the ages of 70 years and 85 years.

For trigeminal nerve stimulation (TNS), the method comprises administration of electrical stimulation of 50-150 Hz, 50-125 Hz, 60-125 Hz, 60-120 Hz, about 60 Hz, about 120 Hz, about 125 Hz, 1-5 kHz, 2-4 kHz, or about 3 kHz as a biphasic symmetric square waveform in 30 s intervals. The electrical stimulation is applied via electrodes placed to bilaterally stimulate the supraorbital branches of the trigeminal nerve. For example, the electrical stimulation is applied in order to bilaterally stimulate the left and right supraorbital branches of the trigeminal nerve. In some implementations, the electrodes are placed on the left and right side of the forehead. In other implementations, the electrodes are placed over the forehead. In some aspects, the interphase pulse is set at 1 μs. In some aspects, the pulse width is 250 μs. In some implementations, the interval period between nerve stimulation is 30 s. During the transition between the stimulation period (ON) and rest period (OFF), the current can be ramped up/down over 5 seconds to make the transitions more comfortable. The current administered is a level tolerable to the subject that is greater than 0 mA but less than the established maximum safety level (for example, 6 mA at 60 Hz or 120 Hz or 13 mA for 3 kHz). In some implementations, the current administered is 2.0-4.5 mA, 2.9±0.7 mA, 3.1±0.7 mA, or 3.4±0.8 mA.

TNS may be administered offline (which is before the execution of a task) or administered online (which is during the execution/performance of a task). Where TNS is administered online, the subject receives TNS for the entire duration of the task. The task may involve reaching with perturbed hand visual feedback or some other physical movement in response to an instruction (whether visual or oral). Therapeutics effects observed in TNS have been shown to require weeks or months of treatment. Therefore, the efficacy of the method can be increased by increasing the session length or applying multiple sessions across several days of TNS.

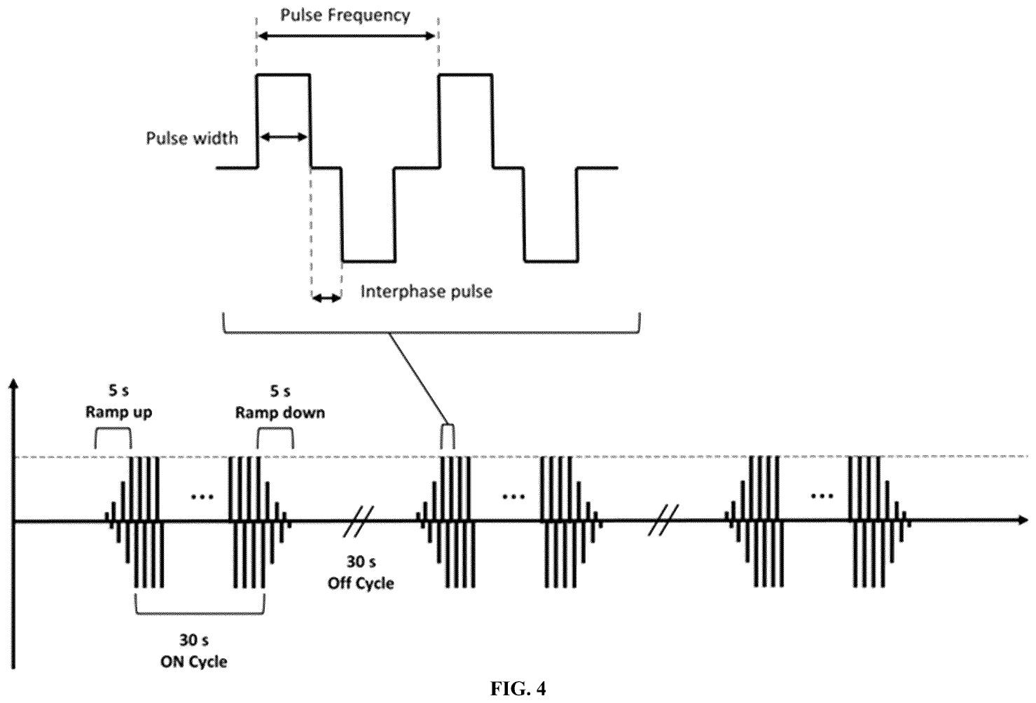

In some implementations, TNS is applied using two round, 3.2 cm diameter, surface electrodes placed on either side of the forehead to bilaterally stimulate the supraorbital branches of the trigeminal nerve. Before the electrodes' placement, the subject's forehead is cleaned with an alcohol prep pad to reduce the impedance between the electrode and the skin. FIG. 4 depicts the stimulation parameter of the described method to stimulate the trigeminal nerves or the occipital nerves for improving motor learning. A biphasic symmetric square waveform was delivered using a peripheral nerve stimulator approved for human research (DS8R, Digitimer Ltd.). The stimulator was triggered using a function generator (AFG 3022B, Tektronix Ltd.), controlled by a custom graphical user interphase written in MATLAB (The MathWorks, Inc.) that also allowed setting stimulation parameters such as the frequency, current amplitude, and pulse width. TNS was delivered in cycles of 30 s ON and 30 s OFF. The current was ramped up/down over 5 s to make the ON/OFF transitions more comfortable.

In some implementations of TNS, the pulse width of TNS is 250 μs, and interphase interval is 1 μs. In such implementations, the electrical stimulation is between 60 Hz and 120 Hz. In other implementations, the pulse width of TNS is 250 μs, and the electrical stimulation is about 3 kHz.

For occipital nerve stimulation (ONS), the method comprises administration of electrical stimulation of 1-5 kHz, 2-4 kHz, or about 3 kHz as a biphasic symmetric square waveform in 30 s intervals. Electrodes for delivering electrical stimulation are placed at the back of the subject's head to target the bilateral branches of the greater occipital nerve. In some embodiments, the electrodes are placed to target the C2/C3 dermatomes. In some aspects, the interphase pulse is set at 1 μs. In some aspects, the pulse width is 250 μs. In some implementations, the interval period between nerve stimulation is 30 s. During the transition between the stimulation period (ON) and rest period (OFF), the current can be ramped up/down over 5 seconds to make the transitions more comfortable. The current administered is a level tolerable to the subject that is greater than 2 mA but less than the established maximum safety level (for example, 16 mA at 3 kHz). In some implementations, the current administered is 9.5-17 mA, 12.8±3.0 mA, or 14.0±2.8 mA.

In some implementations, occipital nerve stimulation (ONS) is administered offline. In some implementations, ONS is administered with TNS, and both nerves are stimulated offline.

In some implementations, ONS was applied using two round 1.5 in (3.81 cm) diameter carbon conductive rubber electrodes (Caputron Medical Products LLC) combined with soaked-saline sponges. Rubber electrodes were placed 2 cm laterally from the occipital protuberance (Inion) targeting the bilateral branches of the greater occipital nerve and C2/C3 dermatomes. Each sponge was soaked with 6 ml of 0.9% sodium chloride solution (saline). A biphasic symmetric square waveform was delivered using a peripheral nerve stimulator approved for human research (DS8R, Digitimer Ltd.). The stimulator was triggered using a function generator (AFG 3022B, Tektronix Ltd.), controlled by a custom graphical user interphase written in MATLAB (The MathWorks, Inc.) that also allowed setting stimulation parameters such as the frequency, current amplitude, and pulse width. ONS was delivered in cycles of 30 s ON and 30 s OFF. The current was ramped up/down over 5 s to make the ON/OFF transitions more comfortable.

In some implementations of ONS, for example where electrical stimulation is about 3 kHz, the pulse width of 50 μs.

The Examples herein demonstrate the effectiveness of single-session transcutaneous electrical stimulation of the trigeminal and occipital nerves on visuomotor adaptation in healthy adults. As such, these neuromodulatory techniques can use useful as an adjuvant to conventional neurorehabilitation of sensorimotor dysfunction. TNS and ONS parameters and protocols disclosed herein can be optimized for improving sensorimotor performance in older adults, augmenting conventional neurorehabilitation, and enhancing human sensorimotor performance in the industrial, athletic, military, and performing arts settings.

TNS can modulate the rate at which a motor task can be learned. Offline TNS using a 60 Hz frequency slowed down the learning of a visuomotor rotation task, compared to sham TNS. This behavioral observation might be explained by the ability of TNS at 60 Hz to alter high or low levels of tonic firing of neurons in the Locus Coeruleus (LC), which has been shown to be associated with poor performance. On the other hand, when TNS at 60 Hz was delivered online, we observed much faster learning compared to offline TNS using the same frequency. This timing-dependent effect suggests different neural mechanisms may be at play during offline vs online TNS. Although Vagus Nerve Stimulation (VNS) has been shown to alter LC activity within milliseconds in animal models, fMRI studies in humans designed to assess brain areas activated by Transcutaneous Auricular Vagus Nerve Stimulation (taVNS) have shown that maximum activation of the Nucleus Tractus Solitarius (NTS) and other brain regions happened several minutes post-stimulation. This suggests that the activation of brain stem nuclei and subsequent neurotransmitter release is a more suitable mechanism for offline stimulation. On the other hand, the effects produced by online stimulation might then be explained by a different mechanism, i.e., increased cortical excitability, which has been postulated to underlie transcranial effects. In support of this idea, simulation studies have suggested that part of the current applied by supraorbital TNS can pass to frontal areas and affect cortical excitability driving behavioral changes.

On the other hand, participants that received offline 3 kHz TNS showed more rapid learning compared to those receiving sham TNS. Similar to the rationale for offline 60 Hz TNS, it is possible that 3 kHz TNS might have optimized LC activity by facilitating phasic LC firing, which has been linked to optimal performance. It is important to note that while the results of our studies employing 60 Hz TNS supports stimulation and timing-dependent effects, neither online nor offline stimulation at 60 Hz resulted in significantly faster learning than sham stimulation. In contrast, participants receiving the offline 3 kHz protocol demonstrated significantly faster learning than those receiving the sham protocol, suggesting it is a better candidate for TNS modulation.

The results also indicate that both the frequency and timing of TNS can influence rates of motor learning in healthy adults. This suggests that optimization of one or both parameters could potentially increase learning rates, which would provide new avenues for enhancing performance in healthy individuals and augmenting rehabilitation in patients with sensorimotor dysfunction resulting from stroke or other neurological disorders.

The Examples suggest that ONS might be more suitable for enhancing learning. ONS showed the fastest adaptation rate among groups. Combining ONS and TNS resulted in more rapid learning than TNS and sham but showed slower learning rates than ONS, which suggest no synergistic effects in combining ONS and TNS. Surprisingly, TNS was associated with the slowest learning rates among active groups, which was unexpected due to the encouraging results observed in experiment 1B, and was even associated with slightly lower learning rates than sham. This surprising result relates to a subgroup of sham group participants that perceived and experienced performance improvement due to the stimulation showed similar learning rates compared to the fastest group, which suggests a placebo effect. Removing this subgroup from the overall sham resulted in faster learning for TNS, similar to the results shown in experiment 1B.

Though the magnitude of the effects on motor learning behavior were modest, the magnitude may be due to a floor effect on visuomotor learning in healthy young adults. The degree of visuomotor adaptation is reduced and more variable in older adults. Thus, an older population can gain greater benefits to the disclosed nerve stimulation methods.

The Examples also show that TNS and ONS for a single session of approximately 20 minutes duration (10 min effective) were safe and tolerable. By using Kilohertz Electrical Stimulation (KES), the submaximal tolerable threshold reaches higher intensities and can be delivered safely. Adverse events were in line with what has been reported in the literature where headaches and dizziness were the most common complaints but were only observed in a relatively small percentage of participants. In just 2 cases, subjects felt lightheaded, and the stimulation was stopped. However, in both cases, participants did not disclose previous fainting episodes which were part of the exclusion criteria. Thus, fainting and vasovagal syncope episode conditions should continue to be excluded to avoid these episodes.

### EXAMPLES

The present disclosure is further illustrated by the following examples that should not be construed as limiting. The contents of all references, patents, and published patent applications cited throughout this application, as well as the Figures, are incorporated herein by reference in their entirety for all purposes.

### Example 1 Methods

The following describes the common methods employed in the studies of Examples 2-7 to investigate effects of trigeminal nerve stimulation on motor learning. Each study exhibited variations in the experimental design (e.g., stimulation delivered offline v/s online, different stimulation parameters, etc.), but the overall methodology employed is similar. For instance, the conducted studies adhered to the same recruitment process, used the same task and apparatus for data collection, and shared data analysis procedures.

### 1. Subject Recruitment

Right-handed individuals without prior history of neurological or psychiatric disorders participated in this series of experiments. All potential candidates completed an online questionnaire to determine eligibility for the study. Written consent was obtained from all participants in accordance with the Declaration of Helsinki. All subjects declared themselves as right-handed, but their handedness was also assessed using the Edinburgh Handedness Inventory-short form. All participants reported having normal or corrected-to-normal vision. Eligible individuals participated in only one experiment. Monetary compensation was provided to participants for their involvement in a given study. All experimental protocols received approval from the Institutional Review Board at Arizona State University.

These studies were designed as single-blind randomized sham-controlled experiment. Block randomization was used to ensure equal sample sizes across groups using Sealed Envelope™

### 2. Apparatus

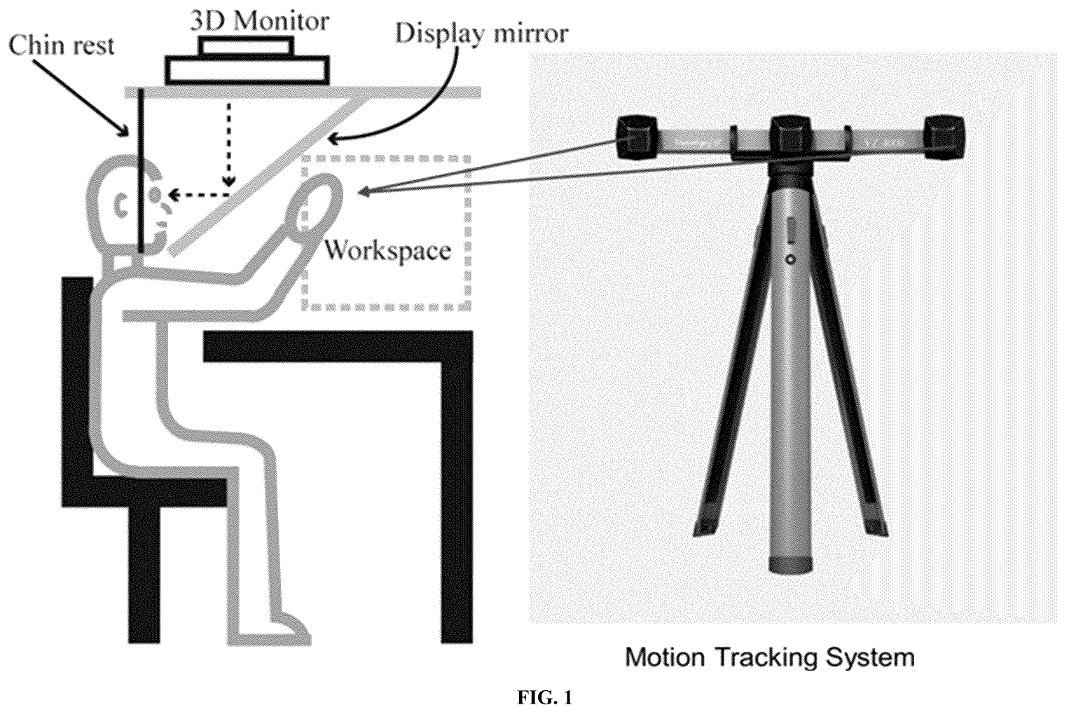

FIG. 1 shows the setup used for these experiments. Participants performed visually-guided reaching movements within a semi-immersive 3D virtual reality (VR) environment. A motion tracking system (Visualeyez VZ-3000; Phoenix Technologies, Inc.) was used to track arm movements. To this end, an LED sensor was attached to the right index finger of each participant. Visual images rendered in Vizard 3.0 (WorldViz Inc.) were displayed on a stereoscopic 3D monitor (Dimension Technologies Inc.) and projected onto a mirror that was embedded within a metal plate oriented at a 45° angle with respect to the monitor. During the experiment, participants were seated with their heads positioned on a chin rest and their eyes aligned with the center of the mirror. To prevent the arm from being directly viewed, movements were performed behind the metal plate, but visual feedback of the fingertip was provided to the participants in the form of a virtual cursor that was projected onto the mirror. Participants across all experiments used this apparatus and performed the task in the same room under the same light conditions.

### 3. Behavioral Paradigm

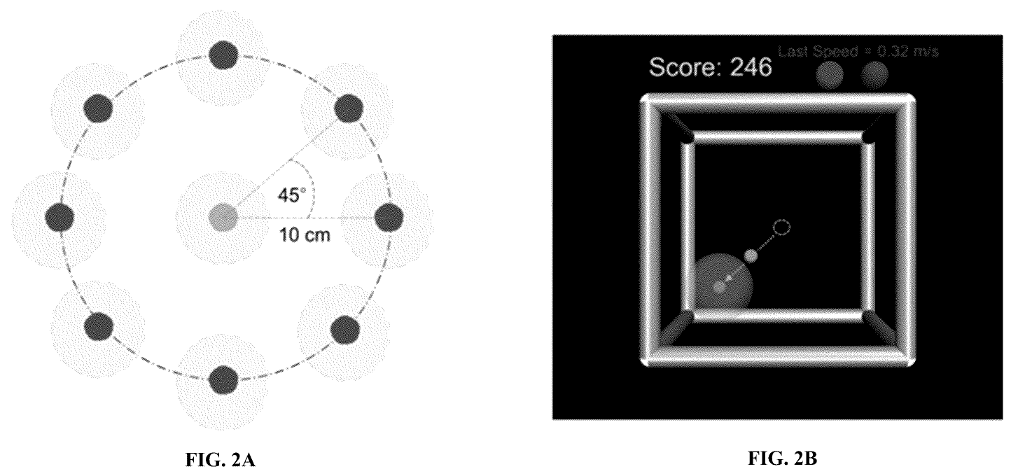

Participants performed a visuomotor rotation task, which requires gradually adapting to an imposed discrepancy between hand motion and corresponding visual feedback (J. W. Krakauer and P. Mazzoni, “Human sensorimotor learning: Adaptation, skill, and beyond,” Current Opinion in Neurobiology, vol. 21, no. 4, pp. 636-644, 2011). To this end, center-out reaching movements were made to eight targets, that appeared one at a time in a pseudorandom order, located 10 cm away from the starting position and 45° apart within a vertical plane (FIG. 2A). Some cues that facilitated depth perception within the 3D environment were implemented. For instance, targets were surrounded by a yellow transparent sphere and appeared inside a cage (FIG. 2B). Subjects were instructed to move as fast and accurately as possible and received visual feedback of their movements as well as auditory feedback for correctly hitting the target. To ensure that participants used approximately the same range of movement velocities, during the trial they also received visual feedback about movement speed and were encouraged to maintain a peak velocity that was greater than or equal to 0.5 m/s. Feedback about movement accuracy was also provided using a point-based scoring system designed to make the task more engaging. Additionally, participants were encouraged to take a break every 25 trials to ameliorate the fatigue in the right arm. To this end, a warning sign appeared on the screen with the message ‘Take a break’. Resting periods could be more frequent if needed.

Although the specific design varied across the experiments, they all followed a similar structure. The study sessions began with a familiarization block, where participants were instructed on how to perform the task. Subsequently, the task progressed through the three main blocks of trials in a standard visuomotor rotation paradigm: Baseline, rotation, and washout (V. Della-Maggiore, S. M. Landi, and J. I. Villalta, “Sensorimotor adaptation: Multiple forms of plasticity in motor circuits,” Neuroscientist, vol. 21, no. 2, pp. 109-125, 2015).

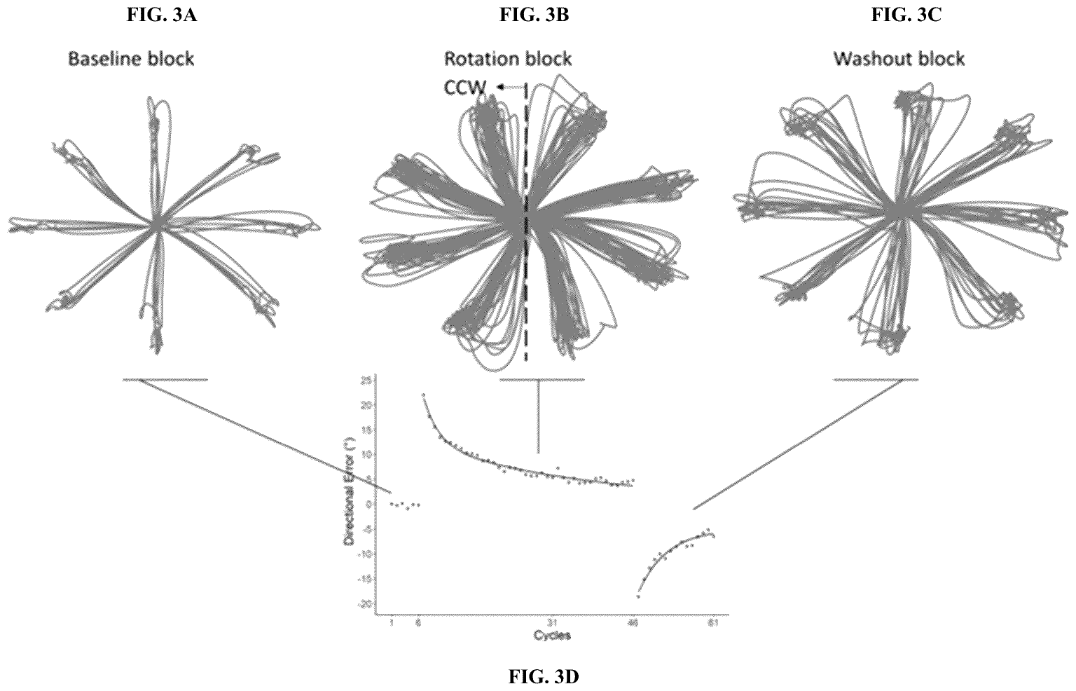

FIG. 3 shows hand trajectories on a trial-by-trial basis and the expected error profile during a visuomotor task. During the baseline block, subjects performed reaching movements with veridical visual feedback, meaning their actual movements matched the visual feedback provided. As expected, arm trajectories were characterized by mostly straight paths to the targets with approximately zero error (FIG. 3A). In the rotation block, which represented the learning phase of the experiment, participants encountered a 30° counterclockwise (CCW) rotation of hand visual feedback. This perturbation produced a mismatch between the actual movement and the visual feedback, resulting in errors in initial movement direction. Hand trajectories pointed toward the cue target location during the early trials, but as the block progressed, trajectories gradually rotated toward the direction that counteracted the rotated visual feedback (FIG. 3B). The directional error produced by the rotation decayed exponentially, converging toward baseline levels. Finally, in the washout block, the rotation of the visual feedback was removed. However, subjects still experienced a discrepancy between the movements and the visual feedback leading to errors in the opposite direction, known as ‘aftereffects’. These aftereffects occurred due to the novel mapping between movement and visual feedback that was learned in the rotation block. Thus, in the washout block initial trajectories pointed toward the learned direction that counteracted the rotation in the previous block. After several movements were executed, hand trajectories gradually rotated toward the actual target direction, restoring the original mapping (FIG. 3C). Thus, the error over time gradually decayed in an exponential fashion that converged to baseline levels. Note that participants did not receive any prior information about the introduction or removal of the rotated visual feedback, nor were they provided with strategies to counteract it.

The experiment was controlled using a custom program designed in LabVIEW (National Instruments Corp.) that allowed the record of kinematic data from the motion tracker and the export of the data into MATLAB files for further analysis. Additionally, the LabVIEW program controlled experimental parameters such as the number of trials, rotation angle, number of targets, etc., and provided the coordinates of the targets to be displayed in the 3D environment.

### 4. Stimulation Protocol

a. TNS Intervention

TNS was applied using two round (diameter: 3.2 cm) surface electrodes (Axelgaard Manufacturing Co., Ltd.) placed on either side of the forehead to bilaterally stimulate the supraorbital branches of the trigeminal nerve. Before the electrodes' placement, the forehead was cleaned using an alcohol prep pad to reduce the impedance between the electrode and the skin. The stimulation was delivered using a peripheral nerve stimulator approved for human research (DS8R, Digitimer Ltd.). The stimulator was triggered using a function generator (AFG 3022B, Tektronix Ltd.), controlled by a custom Graphical User Interface (GUI) written in MATLAB that also allowed setting stimulation parameters such as the frequency, current, and pulse width, among others. FIG. 4 shows a biphasic symmetric square waveform used for TNS. Parameters such as pulse frequency and pulse width were different depending on the corresponding experiment. The interphase pulse was set at 1 μs for all studies. All stimulation protocols were delivered in cycles of 30 seconds ON and 30 seconds OFF, similar to previous studies. During the ON periods, the current was ramped up/down over 5 seconds to make the ON/OFF transitions more comfortable.

Before the stimulation session began, participants were told that a comfortable level of stimulation would be selected, and they might or might not feel it. Following this, the experimenter gradually increased the current until it reached either 1) a tolerable level that did not produce pain or discomfort or 2) an established maximum safety level. Once the intensity level was determined, it was used throughout the experiment, but participants could still request to reduce or stop the stimulation at any time.

### 5. Sham Intervention

For the sham group, electrodes were placed in the same location as the experimental groups, but no stimulation was applied. However, the stimulator emitted the same clicking sounds as when the current was delivered. In all experiments, immediately after the stimulation session was completed (either active or sham), subjects were asked whether they felt the stimulation and, if so, the location of the sensations they experienced.

### 6. Post Study Survey

An online post-study survey was conducted using Google Forms to assess participants' overall experience during the study. The survey aimed to gather feedback on various aspects of the experiments such as discomfort level, side effects experienced, and attention level during the task.

Participants were asked to rate their comfort level during the experiment on a scale of 1 to 10, with 1 indicating feeling uncomfortable and anxious, and 10 indicating feeling very relaxed. They were also requested to rate the level of discomfort or pain experienced during the experiment, if any, using a 1-10 scale. A rating of 1 represented barely noticeable discomfort, while a rating of 10 indicated unbearable discomfort.

Also, participants were asked about potential side effects observed in previous studies that the stimulation might evoke. Specifically, they were asked about headaches, blurry vision, dizziness, and skin itching at the electrode site. If participants had experienced any of these effects, they were instructed to rate their discomfort on a scale of 1 to 10, where 1 represented barely noticeable discomfort, and 10 represented unbearable discomfort.

Finally, participants were also asked to rate their level of relaxation using a 1-10 scale, where 1 indicated mild relaxation, and 10 represented a deep trance-like or meditative state. Similarly, they were asked to rate their attention level during the task on a scale of 1 to 10, where 1 represented not paying attention and 10 represented being engaged and anticipating stimuli.

### 7. Data Analysis

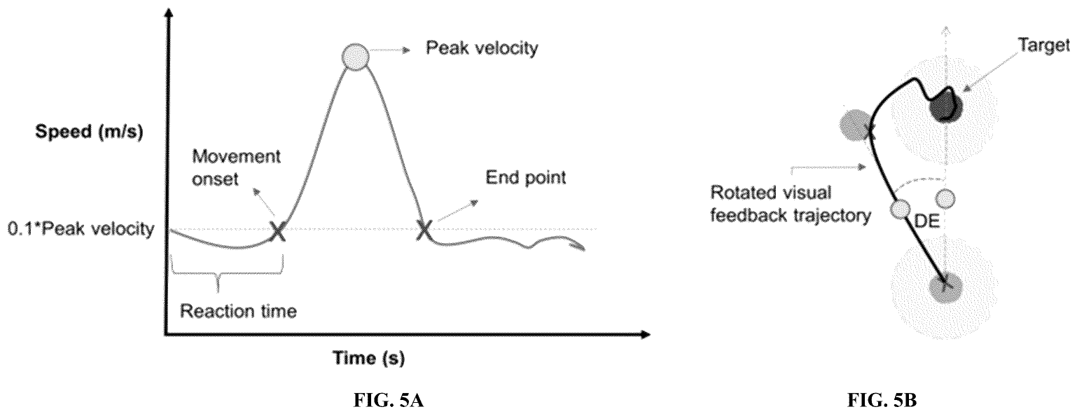

Kinematic analyses were performed in MATLAB (MathWorks, Inc.). Hand movements were sampled at 125 Hz, filtered using a low-pass 2nd order Butterworth filter with a cutoff frequency of 6.25 Hz, and differentiated to obtain hand movement velocities. As shown in FIG. 5A, tangential profiles were used to obtain parameters such as movement onset, Peak Velocity (PV), and Reaction Time (RT). Movement onset was estimated as the first point in time at which the movement exceeded 10% of the PV. The reaction time was measured from the moment the visual target was presented until movement onset. To determine the PV, movement onset, and RT, a semi-automatic script written in MATLAB was used. Each trial was reviewed, and if the movement onset was incorrectly detected, it was adjusted accordingly. Additionally, if the trial presented a velocity profile different from a typical bell shape or the movement trajectory did not follow a straight path, the trial was deleted. Data sets with more than 10% of the total movements deleted were not analyzed further.

Visuomotor performance was quantified by the Directional Error (DE), defined as the angular difference between the rotated visual feedback at PV and the target direction (see FIG. 5B). Cycles were formed by binning 8 consecutive trials to each target direction. Within the cycles, any trial DE, PV, or RT exceeding two standard deviations from the mean was deleted. Subsequent analyses were performed on the binned cycle data.

Individual directional errors during the rotation and washout block were corrected based on the intrinsic bias observed during baseline. Intrinsic bias was calculated based on the procedure. To this end, the mean directional error during baseline for each direction was first calculated. Subsequently, these biases were subtracted from the DE of each direction during the rotation and washout blocks.

Statistical analyses were performed in R (R Core Team, 2014). Following the convention of several previous studies, participants' mean DEs during the rotation block were fitted to a double exponential model (see Eq. 1). This allowed for quantification of the fast and slow learning processes that drive motor adaptation. To this end, a nonlinear least squares procedure based on the Levenberg-Marquardt algorithm was used (nlsLM function in R) to fit the following model:

$$= {C}_{1} {e}^{- α i} + {C}_{2} {e}^{- β i} \qquad ( 1 )$$

where, [symbol] is the estimated directional error during the rotation block, α and β represent the fast and slow learning rate, C₁ and C₂ are the magnitudes of each exponential and i is the i-th cycle during the rotation block. We assumed that α and β>0, α>β and C₁ and C₂>0.

On the other hand, mean DEs for the washout block were fitted to the following single exponential model:

$$= {Ae}^{- γ i} + C \qquad ( 2 )$$

where, [symbol] is the estimated directional error during the washout block, γ represents the forgetting rate, A is the magnitude of the exponential, i is i-th cycle during the washout block and C is a constant. We assumed that A<0 (Error in the opposite direction) and γ>0.

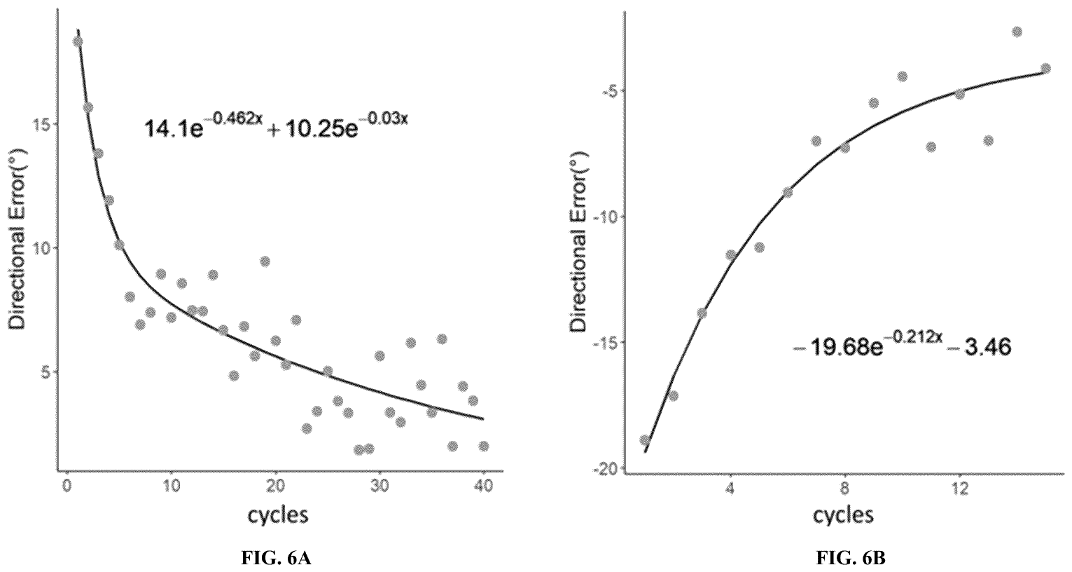

FIGS. 6A and 6B show DEs from a representative subject during the rotation (FIG. 6A) and washout (FIG. 6B) blocks. A double and a single exponential model were fitted to the rotation and washout block, respectively.

To compare the learning and forgetting rates across different groups, various statistical tests were used based on the distribution of the data. Parametric or nonparametric tests were chosen depending on the presence or absence of normality in the data. Normality was assessed using the Shapiro-Wilk test and further verified by examining Q-Q plots. In some cases, bootstrap and permutation tests were utilized to augment these analyses.

Changes in RT and PV were assessed throughout the rotation block, focusing on early and late adaptation phases. To estimate early adaptation, the average RT and PV across the first 15 cycles were calculated for each subject's dataset. Similarly, to estimate late adaptation, the mean values across the last 15 cycles were computed. Comparisons were performed using a two-factor mixed ANOVA (between-subjects factor: experimental groups; within-subjects factor: early and late adaptation phase). A similar approach was used for the washout block. The early phase of the deadaptation was estimated using the average of the first 6 cycles, while the late phase was estimated based on the mean of the last 6 cycles. A two-factor mixed ANOVA was used on the averaged data (between-subjects factor: experimental groups; within-subjects factor: early and late de-adaptation phase). All p-values were adjusted using the Bonferroni correction.

Correlations between learning rate coefficients and attention levels obtained from the post study survey were also explored, as were correlations between the rate coefficients and the amounts of current received.

For all statistical tests, a p-value<0.05 was considered as significant.

### Example 2 Effects of Offline TNS on Visuomotor Learning

Two studies were developed to assess the effects of single-session offline TNS on motor learning in healthy adults. Each study focused on investigating a specific stimulation protocol. In experiment 1A, clinically tested frequencies, i.e., 120 and 60 Hz, were used. In experiment 1, a novel 3 kHz TNS protocol was used. These two pilot studies provide preliminary evidence of the effects of single-session offline TNS on visuomotor adaptation. Clinically tested frequencies and an experimental protocol in the kilohertz range aimed were examined to characterize which provides the best option to enhance motor learning.

### 1. Experiment 1A: Offline TNS Using 120 Hz and 60 Hz

A total of sixty-seven (67) participants were recruited for this study. All of them were self-reported as right-handed; however, three participants were classified as mixed-handed according to the Edinburgh Handedness Inventory. Two of them scored 50, and one scored 37.5, which is below the threshold of 62.5 for right-handedness. Despite this classification, all three participants were included in the analysis. Four data sets were discarded: one (1) participant abandoned in the middle of the experiment due to a conflict in her schedule, and three (3) subjects had noisy kinematic data, resulting in the removal of over 10% of the movements. As described in Example 1, subjects whose data had more than 10% of the movements removed during the data cleaning process were not included in the analysis. The remaining sixty-three (63) young adults (18-36 y/o; 22.75±4.6 y/o; 32 female and 31 men) were randomly assigned to one of three different groups: 120 Hz, 60 Hz, and sham.

A different cohort of forty-three (43) healthy participants was recruited for this study. Subjects were self-reported and classified as right-handed by the Edinburgh Handedness Inventory (score>62.5). Data from one subject was discarded because she was not capable to complete the experiment due to light-headiness after receiving 3 minutes of stimulation. She had fainted once in the past and had a family history of fainting which was not reported, even though the consent form and eligibility criteria survey explicitly asked for it. The participant recovered fully after stopping the stimulation. The remaining 42 subjects (18 40 y/o; 23.4±4.6 y/o; 10 female and 32 male) were randomly assigned to one of two different groups: 3 kHz stimulation and sham.

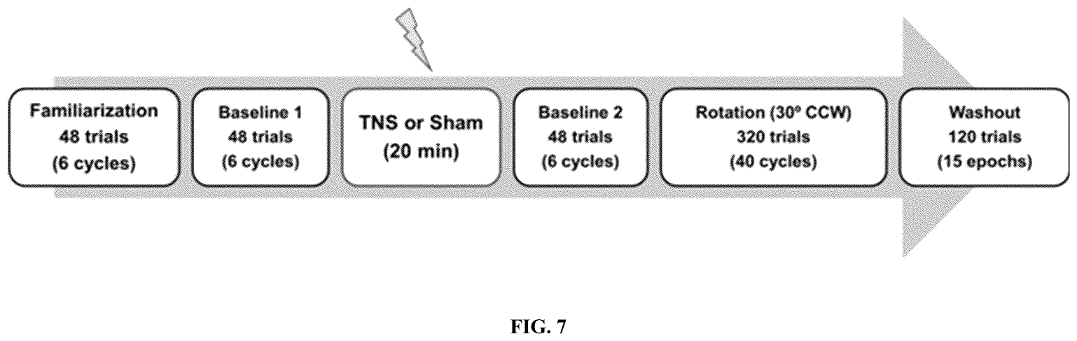

FIG. 7 shows the experimental design for Experiments 1A and 1B. Participants performed a visuomotor rotation task using the apparatus described in Example 1. The experiment began with a familiarization block, followed by one baseline block of 48 trials performed with veridical visual feedback. After the baseline block, subjects received either TNS or sham for 20 minutes under resting conditions. During this period, they viewed images of neutral valence and arousal (131 images with mean valence scores: 5.17±0.4 and mean arousal scores: 3.36±0.76) obtained from the International Affective Picture System (IAPS), in order to minimize variations in emotional states across participants. Each image was displayed for 5 seconds followed by a black screen with a white crosshair in the center for 4.2 seconds. Once the stimulation session was completed, subjects were asked to mark in a diagram of the face anatomy where they felt the stimulation. Immediately after, a second baseline block of 48 trials with veridical feedback and no stimulation was performed to determine whether the stimulation affects baseline motor performance. After the second baseline block, a single adaptation block of 320 trials involving a 30° CCW rotation of the hand visual feedback and no stimulation was performed. The experiment ended with a washout block of 120 trials, where the rotation was removed, and no stimulation was applied.

Regarding the stimulation parameters, the frequencies under study in experiment 1A were 60 and 120 Hz, both with a pulse width of 250 μs. On the other hand, for experiment 1B, the stimulation was delivered at 3 kHz with a pulse width of 50 μs. Stimulation over the supraorbital branches of the trigeminal nerve was delivered in cycles of 30 seconds ON and 30 seconds OFF for 20 minutes in both experiments. Therefore, participants received 10 minutes of effective TNS. The current applied was self-determined by each individual and set to a submaximal tolerable threshold. For the sham groups, electrodes were located on the forehead like those of the active groups, but they did not deliver any current.

Questions regarding attention level, side effects, and discomfort described in Example 1 were applied to participants after the experiment. In addition, participants were inquired about the content of the images displayed during the stimulation. To this end, they rated the content of the images in terms of how it made them feel, using a 1-10 scale, where 1 indicated unpleasant, 5 was neutral, and 10 was pleasant.

a. Data Analysis
i. Analyses of Baseline Performance

A two-factor mixed ANOVA was conducted to examine the effects of the experimental groups (between-subjects factor) and pre/poststimulation (within-subjects factor) on baseline performance. For each subject's data set, the mean Reaction Time (RT), Peak Velocity (PV), and Directional Error (DE) across cycles were calculated separately for baseline 1 and 2 and analyzed statistically.

ii. Analyses of Adaptation and Post-Adaptation Performance

During the rotation and washout blocks, RT and PV were compared between early and late adaptation and deadaptation phase using a two-factor mixed ANOVA.

Changes in DE during the rotation and washout block involved different statistical tests for each experiment. For instance, in experiment 1A, α, β, and γ coefficients were not normally distributed (Shapiro-Wilk test, p<0.05). For this reason, the nonparametric Kruskal-Wallis test was used. Pairwise multiple comparisons using the Dunn's test were performed if significant results were found. P-values were adjusted using the Benjamini-Hochberg correction.

b. Results

On average, participants from the 120 Hz and 60 Hz groups received 3.1±0.8 mA and 3.4±0.8 mA, respectively. No differences were found between intensities among groups (T-test, t=−1.24, df=39.68, p=0.22). Men (n=18, 3.7±0.7 mA) were able to tolerate higher stimulation intensities (T-test, t=−3.6, df=37.7, p=0.00091) than women (n=24, 2.9±0.7 mA).

i. Reaction Time

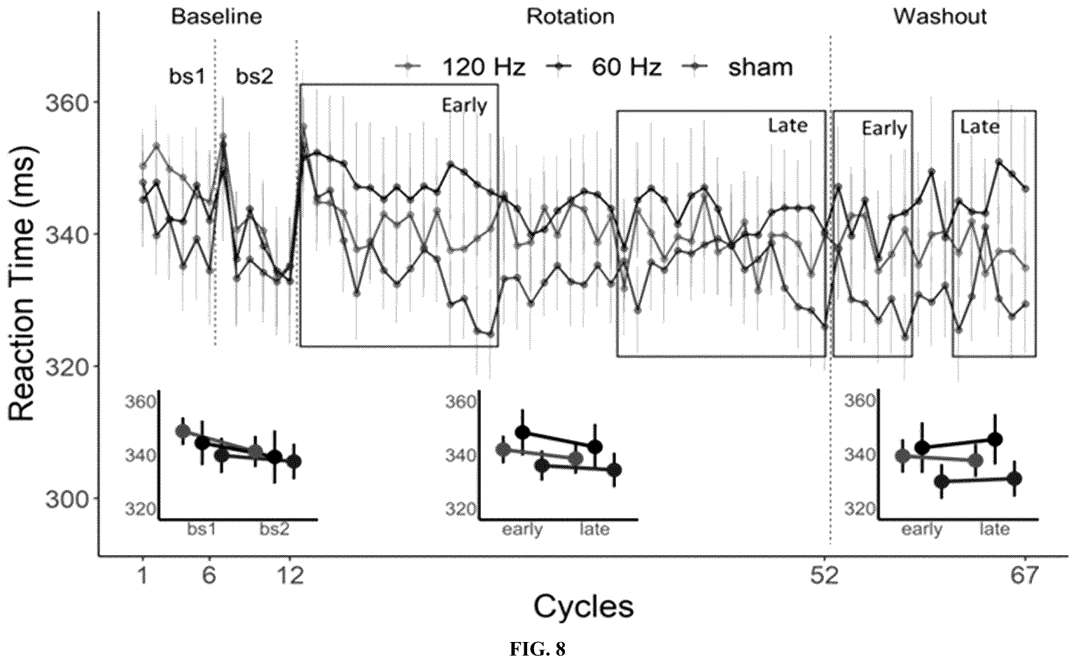

FIG. 8 shows the reaction times for each group throughout the experiment. This figure shows that reaction times fluctuated somewhat from cycle to cycle but did not appear to consistently increase or decrease within or across epochs. In baseline 1, the average reaction time for each group was distributed as follows: 120 Hz (348.8±23.9 ms), 60 Hz (344.5±38.2 ms), and sham (339.8±30.4 ms). In baseline 2, all groups showed a slight reduction in RT. The average RT had the following distribution among groups: 120 Hz (341.3±26.9 ms), 60 Hz (339.2±45.6 ms), and sham (337.5±30.7 ms). A two-factor mixed ANOVA was used to assess the effects of groups and pre/poststimulation on RT. This analysis showed that neither group (F=0.21, p=0.81) nor pre/post-stimulation baselines (F=3.82, p=0.055) had a statistically significant effect on RTs. This two-factor ANOVA revealed that there was not a statistically significant interaction between group and pre/post-stimulation (F=0.36, p=0.701)

During the early adaptation phase (cycles 1-15), the mean reaction time for each group was as follows: 120 Hz (342.0±23.9 ms), 60 Hz (348.4±39.7 ms), and sham (336.0±25.9 ms). In the late adaptation (cycles 25-40), mean RT was the following: 120 Hz (338.6±26.1 ms), 60 Hz (343.0±39.6 ms), and sham (334.4±29.8 ms). A two-factor mixed ANOVA showed no statistically significant differences main effects of groups (F=0.61, p=0.545) or early/late adaptation (F=3.41, p=0.07) on RT. Additionally, no interaction was found between group and early/late adaptation (F=0.34, p=0.72). Finally, for the early washout phase (cycles 1-6), the mean RT was as follows: 120 Hz (348.8±23.9 ms), 60 Hz (344.5±38.2 ms) and sham (339.8±30.4 ms). In the late phase (cycles 9-15), participants presented the following mean RT: 120 Hz (341.5±26.9 ms), 60 Hz (339.2±45.6 ms), and sham (337.5±30.7 ms). A two-factor mixed ANOVA determined that there were no statistically significant main effects of groups (F=0.85, p=0.432) or early/late deadaptation phases (F=0.27, p=0.602) on RT. There was no statistically significant interaction between group and early/late deadaptation.

ii. Peak Velocity

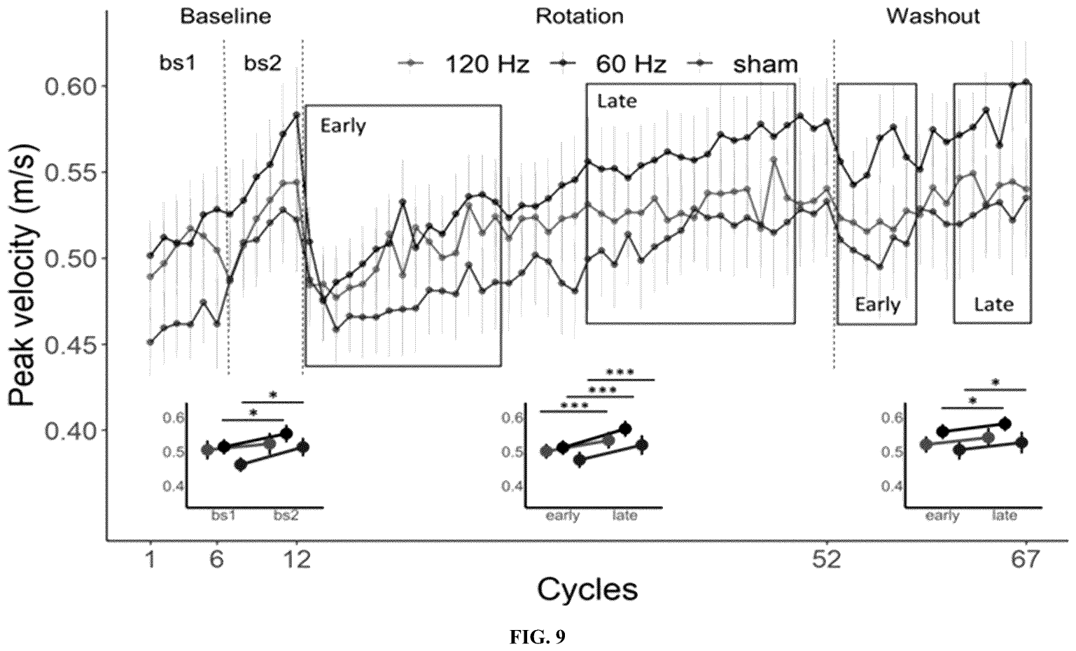

FIG. 9 shows the peak velocities for all groups across the experiment. During baseline 1, the mean PV per group was as follows: 120 Hz (0.50±0.13 m/s), 60 Hz (0.51±0.1 m/s), and sham (0.46±0.098 m/s). In baseline 2, peak velocities tend to increase for all groups. The average PV was distributed as follows: 120 Hz (0.52±0.15 m/s), 60 Hz (0.55±0.12 m/s), and sham (0.51±0.13 m/s). A two-factor mixed ANOVA showed no statistically significant main effects of groups on mean PV (F=0.8, p=0.452), and there was a statistically significant main effect of pre/post-stimulation on mean PV (F=21.99, p<0.001). A pairwise comparison shows that the mean PV of the 60 Hz (p=0.005) and sham (p=0.005) groups were statistically significant between pre/post-stimulation. This two-factor ANOVA also revealed that there was not a statistically significant interaction between groups and pre/poststimulation (F=1.585, p=0.213).

The mean PV for all groups observed an increasing trend from early to late adaptation. During the early phase of the rotation block, the mean PV for each group was as follows: 120 Hz (0.50±0.1 m/s), 60 Hz (0.51±0.1 m/s), and sham (0.48±0.1 m/s). In the late adaptation phase, the mean PV increased for all groups: 120 Hz (0.53±0.1 m/s), 60 Hz (0.57±0.1 m/s), and sham (0.52±0.1 m/s). A two-factor mixed ANOVA showed no statistically significant (F=0.78, p=0.463) main effect of groups on PV. There was a statistically significant effect of early/late adaptation on PV (F=39.57, p<0.001). The pairwise comparison revealed that the mean PV of all groups were statistically significant between early/late adaptation (120 Hz: p=0.006; 60 Hz: p 0.001; sham: p=0.024). The two-factor revealed no interaction between groups and early/late adaptation (F=0.92, p=0.401).

Lastly, in the early deadaptation, the mean PV was as follows: 120 Hz (0.52±0.11 m/s), 60 Hz (0.56±0.10 m/s), and sham (0.51±0.13 m/s). For the late deadaptation, the mean PV per group was the following: 120 Hz (0.54±0.13 m/s), 60 Hz (0.58±0.10 m/s), and sham (0.53±0.15 m/s). Two-factor mixed ANOVA showed no statistically significant main effect of groups on PV (F=1.15, p=0.32). There was a statistically significant main effect of early/late deadaptation on PV (F=13.54, p<0.001). Pairwise comparison showed a statistically significant difference in PV for 60 Hz (p=0.038) and sham (p=0.025) groups between early and late deadaptation. No interaction was found between group and early/late deadaptation (F=0.019, F=0.98).

iii. Directional Error

Directional error during baseline 1 were close to 0° for all groups (120 Hz: 0.10±1.3, 60 Hz: −0.09°±1.3, Sham: −0.1°±1.3) and during baseline 2 (post-stimulation) DEs were also negligible (120 Hz: 0.3°±1.6, 60 Hz: −0.09°±1.6, Sham: −0.1°±1.3). A two-factor mixed ANOVA was used to assess the effects of groups and pre/post-stimulation on DE. This analysis showed that neither of groups (F=0.36, p=0.697) nor pre/poststimulation (F=0.29, p=0.595) had statistically significant effects on DE. Also, there was not statistically significant interaction between groups and pre/poststimulation (F=0.08, p=0.926).

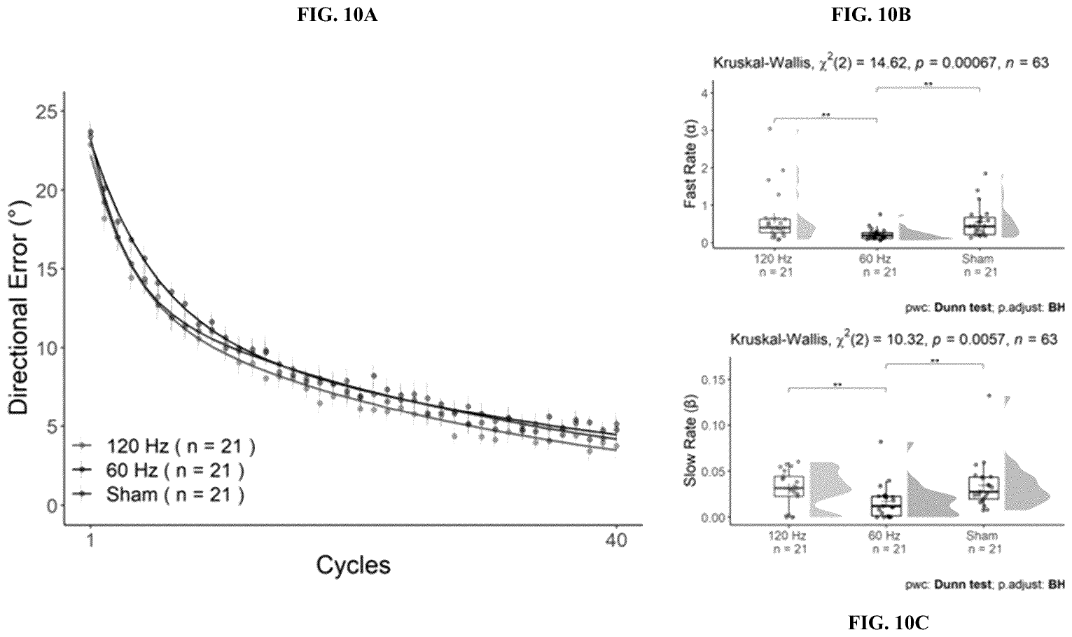

Visuomotor performance (i.e., DE) throughout the rotation block for all groups is illustrated in the form of learning curves in FIG. 10A. Visual inspection of these curves suggests that the 60 Hz group (black bold line) had the slowest adaptation. Analysis of the corresponding learning rates (FIGS. 10B and 10C) revealed a significant difference between groups when fast and slow rates obtained from the double exponential model were analyzed (Kruskal-Wallis test; α: χ2=14.62, df=2, p=0.00067; β: χ2=10.32, df=2, p=0.0057). A post hoc pairwise comparison using the Dunn test revealed that both the fast and slow rates from the 60 Hz group were significantly different from those of the 120 Hz (α: p=0.0019, β: p=0.00065) and sham groups (α: p=0.00049, β: p=0.00045). On the other hand, as shown in FIG. 10A, learning curves were initially similar between the 120 Hz (red bold line) and sham (blue bold line) groups but gradually diverged, with the 120 Hz group exhibiting slightly smaller DEs than sham later in the adaptation block. However, the fast and slow rates comparison showed no statistically significant differences between 120 Hz and Sham (α: p=0.705, β: p=0.906).

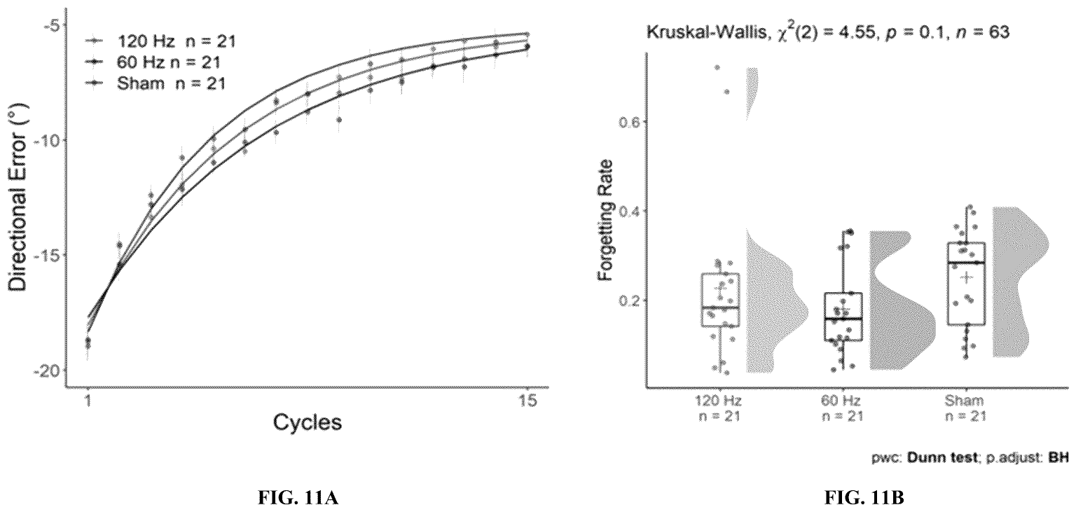

Lastly, FIG. 11A shows the directional error during the washout block. All groups experienced aftereffects, i.e., DEs in the opposite direction reflecting participants learning. A visual inspection of FIG. 11B suggests that the sham group demonstrated faster forgetting rates. However, rate coefficients were highly variable across subjects and as results the rate at which the DEs decayed was similar between groups (KruskalWallis test, χ2=4.55, df=2, p=0.1).

iv. Sham Analysis

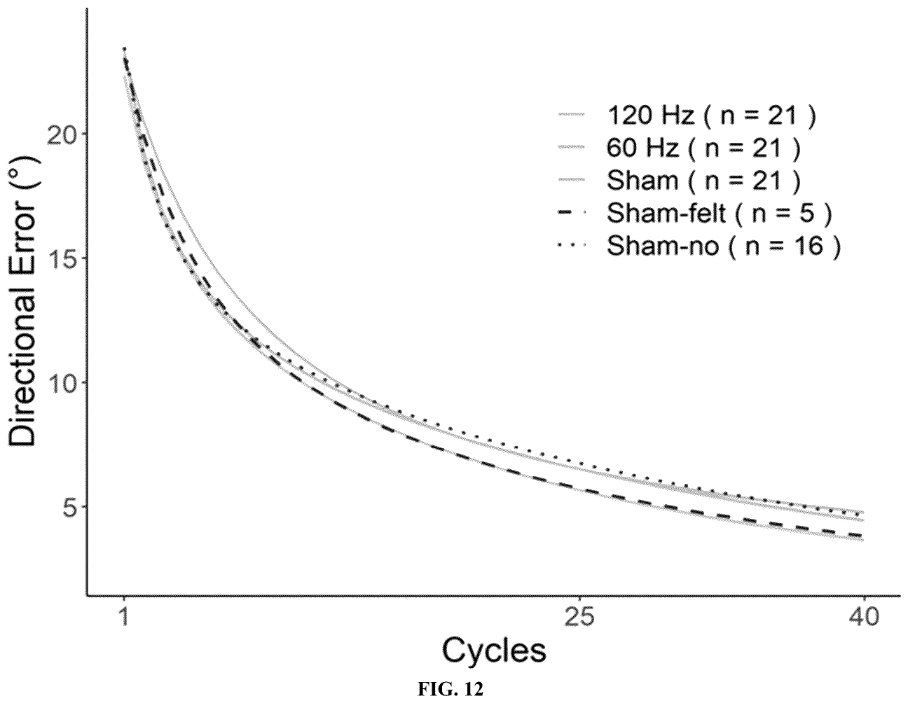

For the sham group, electrodes were placed in the same location as active groups, but no current was applied. After the sham stimulation session, participants were asked to indicate on a diagram of the face where they perceived sensations. Five (5) out of twenty-one (21) subjects from the sham group reported having experienced sensations consistent with the stimulation. As a result, we explored how the perception of received stimulation affected learning rates. To this end, the sham group was split into subjects who perceived the stimulation (Sham-felt; n=5) and who did not (Sham-no; n=16). Interestingly, as shown in FIG. 12, the Sham-felt subjects learned the rotation at a similar rate as those receiving 120 Hz stimulation, which trended toward faster learning. On the contrary, the Sham-no subjects initially performed in a manner similar to the 120 Hz subjects but later converged toward learning rates consistent with 60 Hz stimulation.

v. Post Study Survey

Sixty (60) out of 63 subjects completed the post-study survey. The level of attention reported suggests that all groups were engaged in the task (120 Hz: 7.8/10±2.2, 60 Hz: 8/10±1.8, sham: 7.8/10±2.3). In the 120 Hz, a positive correlation was observed between the attention level and the α coefficient (R=0.48, p=0.032). No other statistically significant correlations between attention level reported and the learning rates were found.

A small group of participants, specifically 10% (6 out of 60) experienced some level of discomfort during the experiment. On average, the discomfort was rated as mild, with an average score of 3.8±2.2 on a scale of 1-10, where 1 represents barely noticeable discomfort and 10 signifies unbearable pain. When examining the individual groups, at least one participant in each experimental group experienced discomfort (120 Hz: n=1, score=4/10; 60 Hz: n=3, score=3/10±1; sham: n=2, score=5/10±4.2).

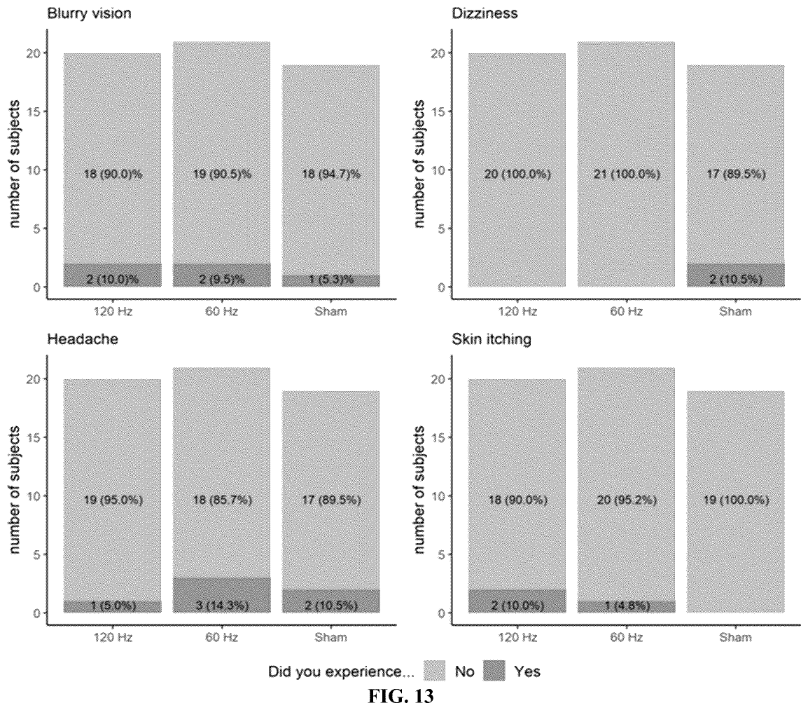

FIG. 13 shows the distribution among groups when consulted for specific side effects they might have experienced during the experiment. In each group of participants, 10% or fewer participants in reported blurred vision (120 Hz: n=2, score=5.5/10±3.5; 60 Hz: n=2, score=3.5/10±1.4; sham: n=1, score=4/10), and approximately 15% or fewer experienced a headache (120 Hz: n=1, score=3/10; 60 Hz: n=3, score=4.3/10±2.5; sham: n=2, score=5/10±1.4). Only 10% of subjects in the sham group (but no other group) reported dizziness (sham: n=2, score=4.5/10±0.7). Finally, skin itching at the electrode site occurred in fewer than 10%, of participants in the stimulation groups (120 Hz: n=2, score=3/10±1.4; 60 Hz: n=1, score=6/10), and in zero participants in the sham group.

### 2. Experiment 1B: Offline TNS Using 3 kHz

A different cohort of forty-three (43) healthy participants was recruited for this study. Subjects were self-reported and classified as right-handed by the Edinburgh Handedness Inventory (score>62.5). Data from one subject was discarded because she was not capable to complete the experiment due to light-headiness after receiving 3 minutes of stimulation. She had fainted once in the past and had a family history of fainting which was not reported, even though the consent form and eligibility criteria survey explicitly asked for it. The participant recovered fully after stopping the stimulation. The remaining 42 subjects (18-40 y/o; 23.4±4.6 y/o; 10 female and 32 male) were randomly assigned to one of two different groups: 3 kHz stimulation and sham.

a. Data Analysis
i. Analyses of Baseline Performance

A two-factor mixed ANOVA was conducted to examine the effects of the experimental groups (between-subjects factor) and pre/poststimulation (within-subjects factor) on baseline performance. For each subject's data set, the mean Reaction Time (RT), Peak Velocity (PV), and Directional Error (DE) across cycles were calculated separately for baseline 1 and 2 and analyzed statistically.

ii. Analyses of Adaptation and Post-Adaptation Performance

During the rotation and washout blocks, RT and PV were compared between early and late adaptation and deadaptation phase using a two-factor mixed ANOVA.

Changes in DE during the rotation and washout block involved different statistical tests for each experiment. For instance, in experiment 1A, α, β, and γ coefficients were not normally distributed (Shapiro-Wilk test, p<0.05). For this reason, the nonparametric Kruskal-Wallis test was used. Pairwise multiple comparisons using the Dunn's test were performed if significant results were found. P-values were adjusted using the Benjamini-Hochberg correction.

The distribution of the α and γ coefficients did not meet the criteria for normality based on the Shapiro-Wilk test (p<0.05). As a result, the Wilcoxon rank-sum test was used for these coefficients. On the other hand, β coefficients were not different from a normal distribution (Shapiro-Wilk test, p<0.05). Hence, a T-test was used.

b. Results

Participants in the 3 kHz group received a mean current of 8.4±1.8 mA during 20 minutes of stimulation. Men (n=17, 8.5±1.9 mA) and women (n=4, 7.9±1.5 mA) within this group were able to tolerate similar intensity levels (Welch test, t=−0.68, df=5.85, p=0.52).

i. Reaction Time

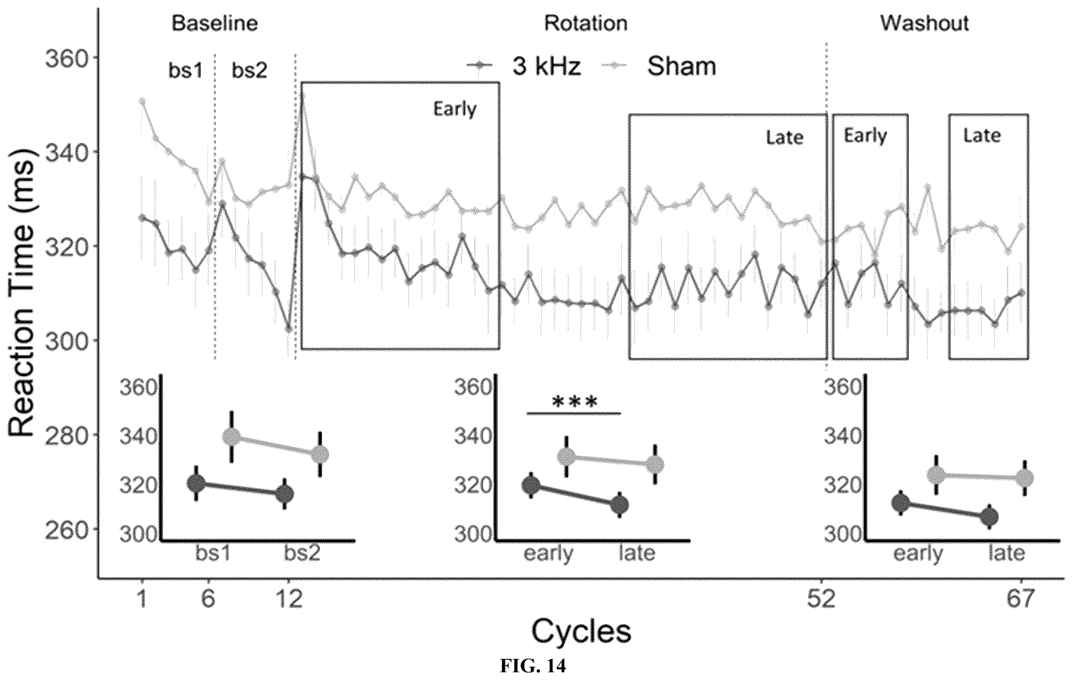

FIG. 14 shows the RT for the 3 kHz and sham group across the experiment. The 3 kHz group showed slightly lower RT than sham in all blocks. During baseline 1, the mean RT was 320.4±33.5 ms and 339.5±48.9 ms for the 3 kHz and sham groups, respectively. In baseline 2, the average for each group was 316.1±29.2 ms for the 3 kHz and 332.3±42.7 ms for the sham. The two-factor mixed ANOVA showed no significant main effects of groups on RT (F=2.21, p=0.145). Although there was a statistically significant main effect of pre/post-stimulation on RT (F=4.53, p=0.039), the p-values corrected by Bonferroni during the pairwise comparison were not statistically significant for the 3 kHz (p=0.233) and sham (p=0.096). Additionally, the analysis showed that there was not a statistically significant interaction between groups and pre/post-stimulation (F=0.285, p=0.596).

During the early phase of the adaptation, participants in the 3 kHz group showed a mean reaction time of 319.6±24.7 ms. For the sham group, the mean RT was 331.2±38.9 ms. In the late phase, the 3 kHz and the sham groups presented a mean RT of 311.6±24.7 ms and 328.1±37.4 ms, respectively. A two-factor mixed ANOVA showed no statistically significant main effect of groups on RT (F=2.09, p=0.156). There was a statistically significant main effect of pre/poststimulation on RT (F=8.13, p=0.007). A post hoc test revealed a statistically significant difference between the early and late adaptation phases for the 3 kHz group (p<0.001) but not for the sham (p=0.373). Also, there was no statistically significant interaction between groups and early/late adaptation (F=1.52, p=0.225).

Finally, mean RT during the early phase of the washout block were 312.4±23.8 ms for the 3 kHz group and 323.8±37.4 ms for the sham group. For the late phase, the mean RT was 306.7±123.6 ms for 3 kHz, and 322.5±33.6 ms for sham. A two-factor mixed ANOVA showed no statistically significant main effects of groups (F=2.24, p=0.143) or early/late deadaptation (F=2.69, p=0.109) on mean RT. This analysis also showed no statistically significant interaction between group and early/late deadaptation.

ii. Peak Velocity

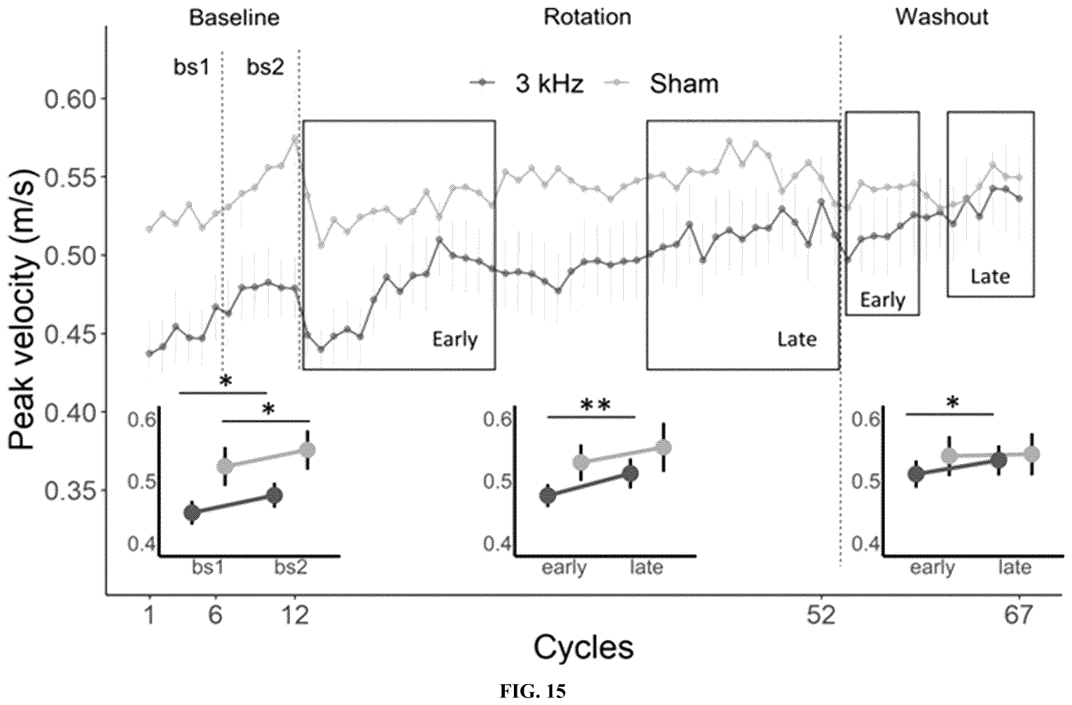

FIG. 15, shows the peak velocity for each group throughout the experiment. It is possible to observe that the PV for the 3 kHz group tended to increase as the experiment progressed. In baseline 1, the mean peak velocity was 0.45±0.09 m/s for the 3 kHz and 0.52±0.14 m/s for the sham. In baseline 2, the mean PV was 0.48±0.09 m/s and 0.55±0.15 m/s for the 3 kHz and sham, respectively. A two-factor mixed ANOVA showed no statistically significant main effects of groups (F=4.06, p=0.051) on PV. There was a statistically significant main effect of pre/post-stimulation (F=11.24, p=0.002) on PV. A post hoc test revealed a statistically significantly different between pre and post-stimulation for both, the 3 kHz (p=0.016) and sham group (p=0.043). Additionally, this analysis also revealed that there was not a statistically significant interaction between group and pre/post-stimulation (F=0.006, p=0.940).

Mean peak velocities during the early phase of the rotation were 0.48±0.08 m/s and 0.53±0.14 m/s for the 3 kHz and sham, respectively. For the late phase, the mean PV for the 3 kHz group was 0.53±0.14 m/s and 0.55±0.18 m/s for sham. The two-factor mixed ANOVA analysis showed no statistically significant main effect of groups on PV (F=1.4, p=0.244). On the contrary, there was a statistically significant main effect of early/late adaptation (F=11.28, p=0.002) on PV. A post hoc multiple comparison revealed a statistically significant difference between early and late adaptation for 3 kHz (p=0.004) but no for the sham (p=0.099). Also, this analysis showed that there was not a statistically significant interaction between groups and early/phase adaptation on PV (F=0.366, p=0.549).

Mean peak velocities for the early deadaptation phase were 0.51±0.10 m/s and 0.54±0.15 m/s for the 3 kHz and the sham, respectively. In the late phase, mean PV were 0.53±0.11 m/s and 0.54±0.16 m/s for the 3 kHz and sham groups, respectively. The two-factor mixed ANOVA showed no statistically significant main effect of groups on PV (F=0.24, p=0.630). Also, there was a statistically significant effect of early/late deadaptation on PV (F=4.84, p=0.034). A post hoc pairwise comparison revealed a statistically significant between early and late deadaptation for the 3 kHz group (p=0.026) but not for the sham group (p=0.655). Also, the analysis showed no statistically significant interaction between group and early/late deadaptation (F=2.735, p=0.106).

iii. Directional Error

Similar to experiment 1A, the DE among all groups was approximately zero during baseline 1 (3 kHz: 0.38°±1.3, Sham: 0.25°±1.3) and baseline 2 (3 kHz: 0.46°±1.4, Sham: 0.006°±1.4). A two-factor mixed ANOVA showed no statistically significant main effects of groups (F=0.56, p=0.458) or pre/post-stimulation (F=0.17, p=0.686) on DE.

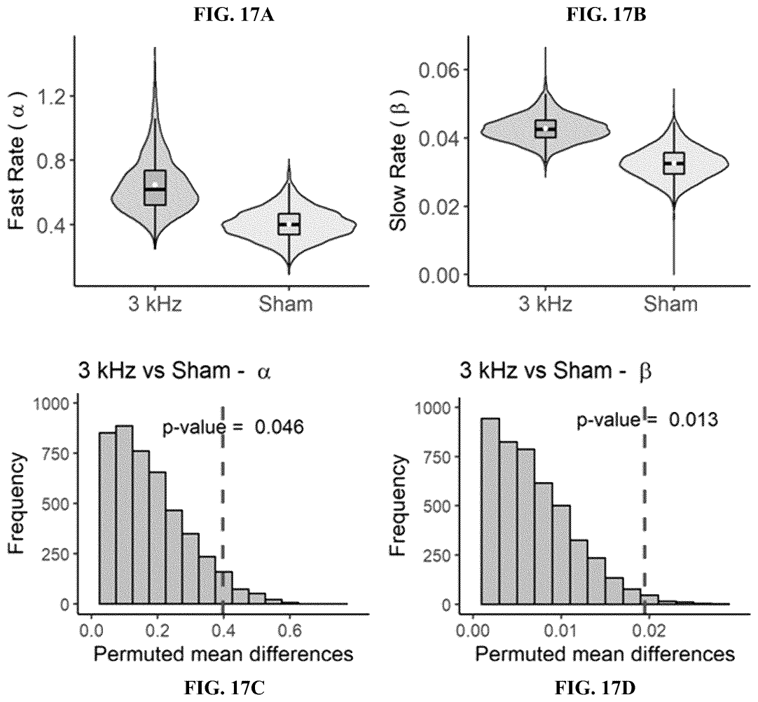

FIG. 17A shows the learning curves obtained after fitting a double exponential model to the average directional error for the 3 kHz (maroon bold line) and sham (orange bold line). The learning curve for the 3 kHz decayed faster than the sham, suggesting enhanced motor adaptation. FIG. 17B shows that the fast rate coefficients appeared to be greater for the 3 kHz than for the sham, with a tendency towards significance (Wilcoxon test, W=294, p=0.066). Similarly, FIG. 17C demonstrates that the slow rate coefficients were significantly greater for the 3 kHz compared to the sham (T-test, t=2.58, p=0.014). Analysis of learning rates were consistent with the learning curves shown in FIG. 17A. Additionally, these results were in line with those provided by the permutation test and bootstrap resampling performed on the fast and slow rate coefficients shown in FIGS. 17C and 17D.

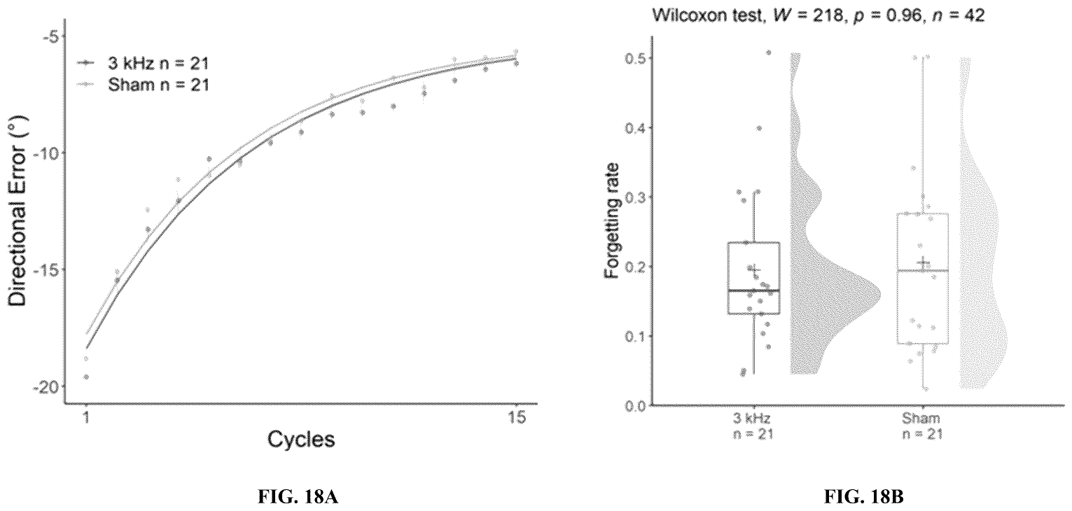

FIG. 18A shows the directional error fitted to a single exponential model during the washout block. Both groups experienced aftereffects, for example, DEs in the opposite direction, reflecting participants learning, but the rate at which the DEs decayed were similar between groups as shown in FIG. 18B (Wilcoxon rank-sum test, W=85, p=0.73).

iv. Sham Analysis

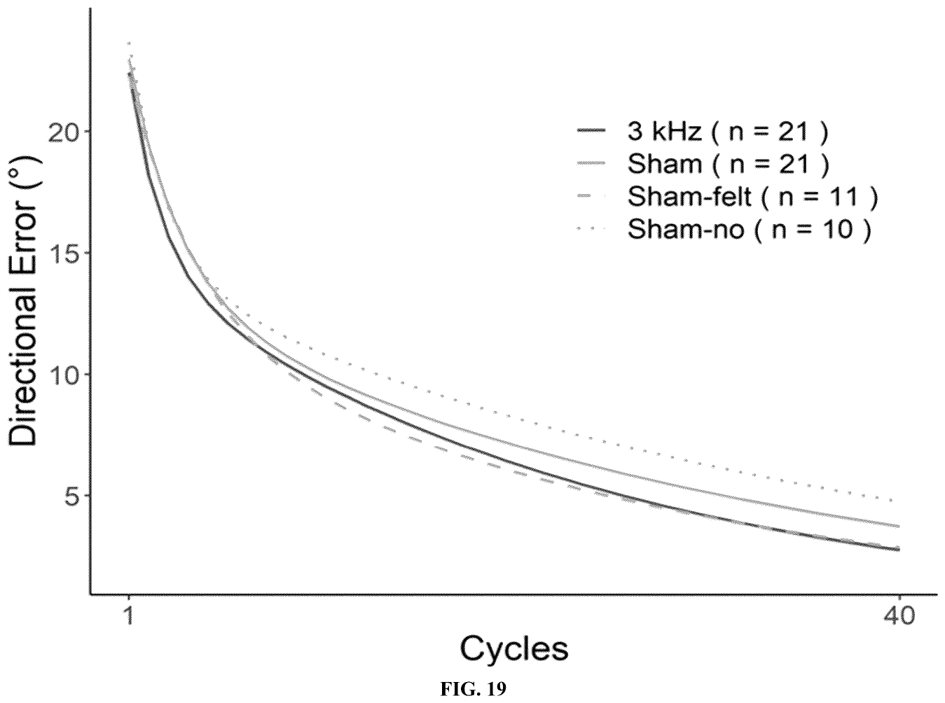

Eleven (11) participants (52%) in the sham group experienced the stimulation even though no current was applied to them. Similar to experiment 1A, new learning curves for the sham group were generated by dissociating the DE of subjects who felt the simulation (Sham-felt; n=11) and those who did not (Sham-no; n=10). FIG. 19 shows that participants who perceived the stimulation learned the rotation to a rate similar to the 3 kHz group. On the other hand, subjects who did not perceive the stimulation adapted slower compared to sham, showing worst visuomotor performance.

v. Post Study Survey

The post-study questionnaire was responded by 41 out of 42 subjects. Both groups reported high attention level during the task performance (3 kHz: 7.7/10±1.9, Sham: 7.7/10±2.3). No correlations between the attention level and the learning rates, and the current received were found.

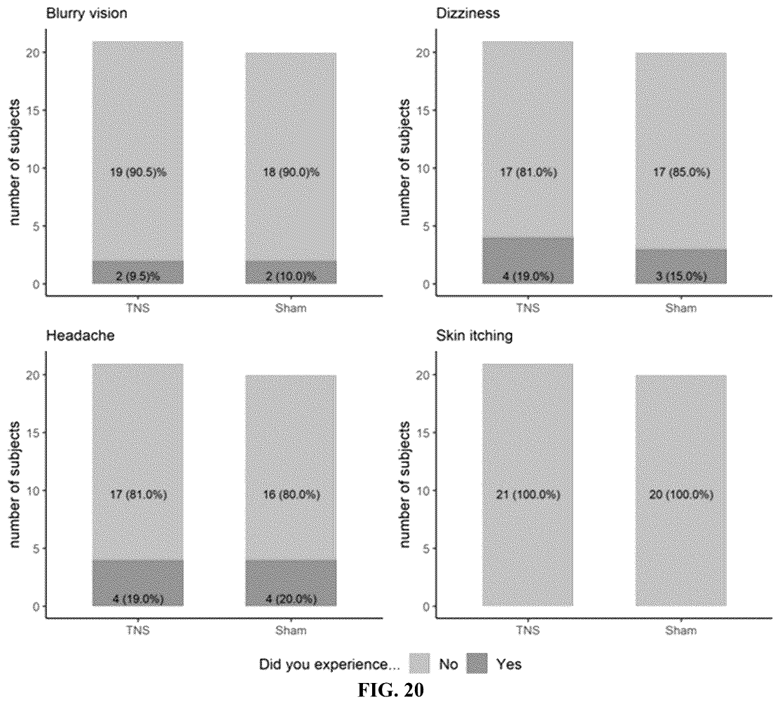

Also, most of the participants rated their comfort level during the experiment with an average of 8.4/10±1.9. However, when asked for some episodes of discomfort or pain, 37% (15 out of 41) reported to have experienced some level of discomfort. From this subgroup, 7 participants belonged to the 3 kHz group and reported an average discomfort level of 3.8/10±2.5. On the other hand, 8 subjects assigned to the sham group reported an average discomfort level of 4.5/10±2.3. Also, approximately 20% of participants or less from both groups experienced a headache (3 kHz: n=3, rate=5.3/10±2.2; Sham: n=3, rate=5.3/10±3.6) and dizziness (3 kHz: n=4, rate=2.8/10±2.2; Sham: n=3, rate=6/10±0) and less than 10% experienced blurry vision (3 kHz: n=2, rate=1.5/10±0.7; Sham: n=2, rate=5/10±4.2) during or after the experiment. Skin itching and other adverse events were not reported. FIG. 20 summarizes these findings.

Additionally, subjects who belonged to the active group reported tingling and pressure in the forehead during the stimulation. Discussion

### 3. Discussion of Results

a. Effects on Learning and Motor Performance

In experiment 1A, 60 and 120 Hz TNS were assessed with the expectation that these frequencies would have enhanced the learning rates during the adaptation block. However, that was not the case. Analysis of the learning curves showed that TNS at 60 Hz slowed the learning, which was more pronounced in the early adaptation phase. Both fast and slow coefficients were smaller and statistically significant compared to the 120 Hz and sham groups, which might suggest that TNS at 60 Hz had a detrimental effect on explicit and implicit mechanisms. On the other hand, the 120 Hz group ended up learning the perturbation slightly faster than the sham. However, changes in directional error were relatively modest and were not statistically significant.

In experiment 1B, TNS applied at 3 kHz was assessed. Examination of the learning curves during the rotation block suggested that the 3 kHz group learned the rotation faster than the sham group. This way, learning rate comparison suggests that 3 kHz might also affect both learning systems. Although only the slow rates were higher and statistically different from sham, the fast rates tended in the same direction (p=0.066). Further analysis using a permutation test showed that fast rates were also different from sham.

In addition, the 3 kHz group showed enhanced RT and PV post-stimulation. During the rotation and washout blocks, the 3 kHz group exhibited a statistically significant increase in PV between early and late adaptation, as well as between early and late deadaptation. Similarly, during the rotation block, the 3 kHz group displayed a reduction in RT between early and late deadaptation. Thus, the 3 kHz group not only showed faster learning rates but also a more substantial reduction in RT and an increase in PV compared with the sham group, suggesting that using 3 kHz may be a superior option compared to the 120 and 60 Hz protocols for future experiments the motor domain.

In both experiments, analysis of participants' directional errors and reaction time revealed that the stimulation did not affect visuomotor performance during the baseline blocks, which involved only veridical visual feedback. On the other hand, we observed in both experiments an increase in the peak velocity after the stimulation/sham session for all groups. The increase in peak velocity in baseline 2 may be attributed to practice. Participants moved faster as they got used to the task and felt more comfortable with the 3D environment. Although having a post-stimulation baseline was a necessary control to determine whether TNS affects motor performance in unperturbed conditions, it might have risked some neuromodulatory effects during those approximately 5 minutes of baseline trials, which could have been used during the early phase of the rotation block. Besides this, we observed effects using 60 Hz and 3 kHz stimulation that altered the rate of learning.

Although the mechanisms behind these effects remain unknown, the Aston-Jones model may provide a possible explanation. This model relates task performance with Locus Coeruleus (LC) activity, similar to the Yerkes-Dodson law that relates performance with arousal using an inverted U shape. Assuming that both frequencies were able to alter LC activity, 60 Hz TNS may disrupt the balance of the tonic firing, resulting in very low or high levels of activity at the LC, which has been linked to poor performance. On the other hand, the 3 kHz protocol may be more likely to induce moderate tonic firing and facilitate phasic firing, which is associated with optimal performance. LC sends projections to several areas of the cortex, including the Prefrontal Cortex (PFC), associated with explicit learning, and the cerebellum with implicit learning. The ability of these stimulation protocols to alter LC activity during a visuomotor rotation task might activate these cortical areas, affecting both learning mechanisms. This perspective offers a potential explanation for the observed effects. However, further investigation needs to be done.

b. Sham Groups Might be Affected by Placebo Effects

In both studies, some participants in the sham group (24% in Exp. 1A and 52% in 1B) perceived the stimulation even though no current was applied. Interestingly, examining the directional error throughout the adaptation block of this sham subgroup, we observed they learned the rotation at a faster pace, similar to the best-performers groups (120 Hz and 3 kHz), suggesting a potential placebo effect. Previous studies in the brain stimulation field have reported strong placebo effects, which have been attributed to various factors such as the use of bulky equipment, buzzing, and noise produced by the equipment, electrode placement, among others. In the protocol used for these examples, subjects in the sham group used the same electrode placement, the stimulator made the same clicking sound every time it was supposed to be active, and the experimenter mimicked the same protocol used for the active groups to look for the submaximal tolerable threshold. All these factors may have contributed to a placebo effect in part of the sham group. On the other hand, participants who did not perceive the stimulation presented the slowest learning among groups. The expectation of receiving a treatment, i.e., stimulation or a drug, has been shown to be relevant in placebo/sham-controlled studies. The fact that this subgroup of the sham did not feel the stimulation might have lowered their expectations of the stimulation, potentially affecting their performance.

c. Stimulation Tolerability

10% of participants in experiment 1A and 37% in 1B reported to have experienced some discomfort during the experiments. Although TNS might have produced some discomfort, it is unlikely because participants were encouraged to select a comfortable intensity but also, they could ask to reduce or stop the stimulation at any time. Additionally, subjects in the sham group also reported discomfort, even though they did not receive any current. Thus, the discomfort is likely the result of arm fatigue due to the multiple arm movements. To clarify the source of the discomfort participants can be asked where the discomfort is located, if any.

During experiment 1B, one participant felt lightheaded after three minutes of TNS at 3 kHz. Unfortunately, the participant did not disclose their history of fainting episodes. Syncope and dizziness has been reported in the VNS literature. Considering the similarity in the mechanisms of action between TNS and VNS, individuals who have experienced fainting or vasovagal syncope were excluded to minimize the risk of such adverse events during the experiment. Despite this event, TNS was well tolerated during the stimulation session in both experiments. Participants in the 3 kHz groups were able to tolerate higher currents than the 120 and 60 Hz groups, which was in line with other studies where frequencies in the kilohertz range were used. Additionally, men tended to choose higher intensities when stimulation was delivered at frequencies of 60 and 120 Hz. This was not observed in experiment 1B, however, gender-wise, the sample was unbalanced with only 4 females compared to 15 males in the 3 kHz group, which makes it difficult to draw conclusions regarding a gender-intensity relationship.

Although the majority of participants did not report any serious adverse events when specifically consulted for headaches, dizziness, blurry vision, and skin itching at the electrode site, a small percentage reported having experienced them. Although headaches and skin itching have been associated with TNS in other studies (J. J. McGough, A. Sturm, J. Cowen, K. Tung, G. C. Salgari, A. F. Leuchter, I. A. Cook, C. A. Sugar, and S. K. Loo, “Double-blind, sham-controlled, pilot study of trigeminal nerve stimulation for attention-deficit/hyperactivity disor-der.” Journal of the American Academy of Child and Adolescent Psychiatry, vol. 58, no. 4, pp. 403-411, 2019; C. M. DeGiorgio, J. Soss, I. A. Cook, D. Markovic, J. Gornbein, D. Mur-ray, S. Oviedo, S. Gordon, G. Corralle-Leyva, C. P. Kealey, and C. N. Heck, “Randomized controlled trial of trigeminal nerve stimulation for drug-resistant epilepsy,” Neurology, vol. 80, no. 9, pp. 786-791, 2013), in these experiments is difficult to associate them exclusively as a side effect of the stimulation because subjects in the sham group also experienced them, even though no current was applied. Other factors such as the experiment duration (2 h) and the fact that the paradigm was performed in a semi-immerse VR environment might have contributed to these adverse events. For example, it is known that VR may produce cybersickness which considers dizziness, blurry vision, and headache as part of the symptoms. This way, the VR environment might have contributed to the appearance of these adverse events.

### 4. Conclusion

Results from both experiments suggest that TNS may exhibit stimulation-dependent effects on visuomotor adaptation, which provides new information on the potential use of TNS in motor applications. Specifically, when TNS was delivered offline for 20 minutes at 60 Hz and 3 kHz, it was found to have the capability of altering the learning rate of a visuomotor rotation task. Interestingly, in one instance, the learning rate was reduced, while in the other, it was enhanced. A potential explanation of these effects can be found in the Aston-Jones model that relates LC activity with task performance. 60 Hz might disrupt tonic firing in the LC which is linked to poor performance. On the contrary, 3 kHz may induce moderate tonic firing and facilitate phasic firing, which is linked to optimal performance. Determining whether these effects were influenced by the frequency or the current intensity of TNS requires further investigation.

### Example 3 Effects of Online TNS on Visuomotor Learning

Example 2 showed that 60 Hz Trigeminal Nerve Stimulation (TNS) had a detrimental effect on learning, while 120 Hz stimulation tended to result in slightly faster learning than sham. In an effort to potentially improve upon previous outcomes and provide a complete characterization of the timing effects of TNS, this Example assesses TNS using clinically tested frequencies (120 and 60 Hz) delivered online during performance of the same visuomotor learning task (designated as Experiment 2). A comparison is made with the results obtained from the offline study in Example 2.

### 5. Participants

Sixty-five (65) subjects were recruited for Experiment 2. All subjects self-reported themselves to be right-handed and were also classified as right-handed by the Edinburgh Handedness Inventory (score>62.5). Two data sets were discarded: one participant complained about eye strain and abandoned the experiment, while an-other participant, assigned to one of the stimulation groups, was stimulated with a different type of surface electrodes that were not consistent with the ones used in this study. This way, 63 subjects (18-32 y/o; 23.2±3.9 y/o; 12 female and 51 male) were considered for analysis. The experimental groups were the same as experiment 1A: 120 Hz, 60 Hz, and sham.

### 6. Study Procedure

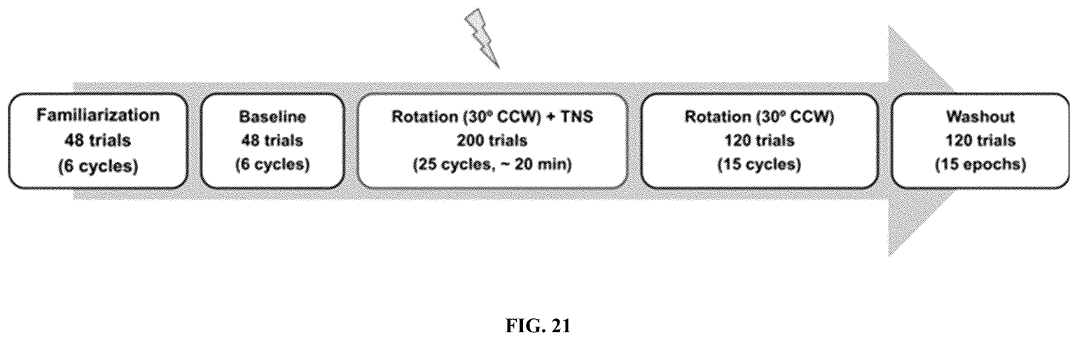

Like experiment 1A and 1B of Example 2, participants begun with a familiarization block fol-lowed by a baseline block of 48 trials to assess their performance under unperturbed conditions. After the baseline block, subjects experienced an adaptation block of 200 trials involving a 30° CCW rotation of hand visual feedback and concurrent TNS or sham. Once this block was completed, subjects were asked if they felt something and, if so, to sketch on a diagram of the face anatomy where they felt experienced sensations. Then, participants performed another 120 trials with rotated feedback but no stimulation. The experiment ended with a washout block of 120 trials, where the rotation was removed, and no stimulation was applied. FIG. 21 shows the experimental design.

### 7. Stimulation Protocol

The same stimulation protocol described in Example 1 was used. The only difference was that TNS was delivered online, concurrently with the task and the stimulation stopped when 200 trials (25 cycles) of the rotation block were completed. Based on pilot data, participants could complete 200 trials in approximately 20 minutes. This way, the dose duration is expected to be comparable to that of experiment 1A.

### 8. Data Analysis

a. Analyses of Baseline Performance

A one-way ANOVA was conducted to compare Directional Error (DE), Reaction Time (RT), and Peak Velocity (PV) between groups during the baseline block. Prior to the analysis, Shapiro-Wilk test confirmed that the distribution of the data was not different from normal distribution (p>0.05), thus satisfying the normality requirements for the ANOVA test.

b. Analyses of Adaptation and Post-Adaptation Performance

DE, RT, and PV during the rotation and washout block were analyzed based on the methods described in Example 1.

Similar to experiment 1A, Kruskal-Wallis test was used to compare learning and forgetting rates because they were not normally distributed (Shapiro-Wilk test p>0.05). Learning curves and rates from experiment 1A, where TNS was applied offline, were compared with the results of this study. To this end, Wilcoxon test was used.

### 9. Results

The stimulation groups self-selected similar electrical currents (60 Hz: 3.3±0.8 mA; 120 Hz: 3.1±0.8 mA). A t-test showed that there was no statistically significant difference between these currents (t=−0.67, df=40, p=0.51). The duration of the stimulation session was approximately 20 minutes (120 Hz: 21±4.2 min, 60 Hz: 22±4.1 min, sham: 21.8±3 min), similar to the offline protocol used in experiment 1A. Additionally, there was no statistically significant differences (T-test, t=−0.35, df=16.45, p=0.73) in the intensity level tolerated by men (n=34, 3.2±0.8 mA) and women (n=8, 3.1±0.5 mA).

On average, participants reported in the post-study survey to have been attentive during the task performance (120 Hz: 8.1/10±1.8, 60 Hz: 8.5/10±1.3, sham: 8.2/10±1.8). When assessing potential correlations between the attention level, the current applied, and the learning rates, no relationships were found.

a. Reaction Time

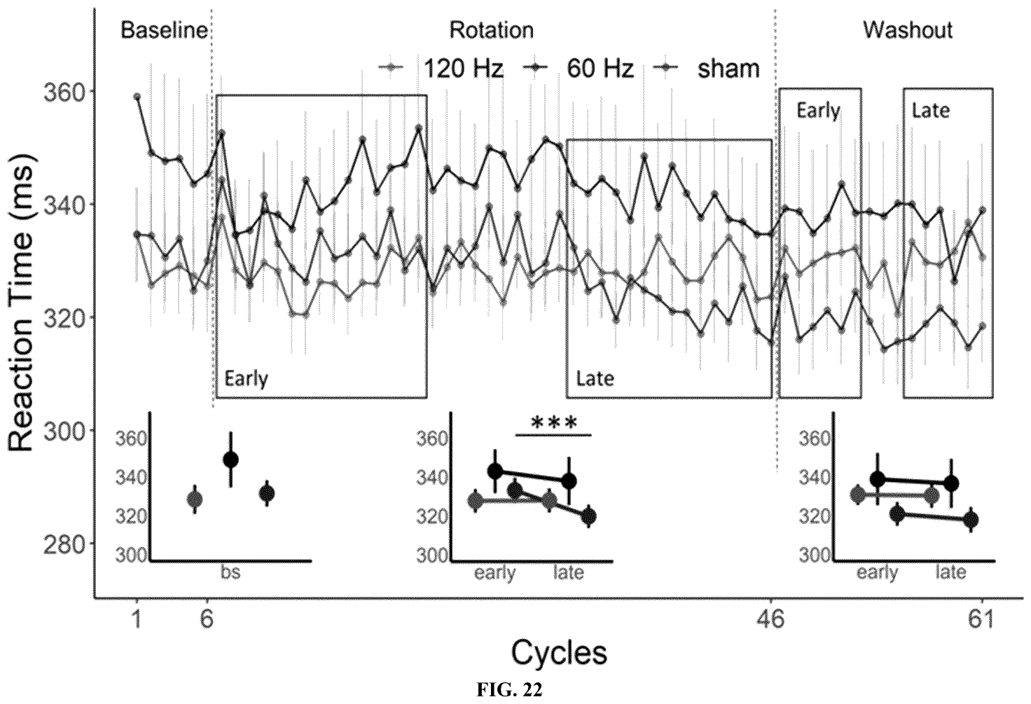

FIG. 22 shows the reaction time during the baseline block. The specific reaction times per group were as follows: 120 Hz (328.3±34.6 ms), 60 Hz (348.8±66.0 ms), and sham (331.4±31.5 ms). A one-way ANOVA revealed no statistically significant differences among groups (F=1.174, p=0.316).

In the early phase of the adaptation, the reaction time reached the following mean values per group: 120 Hz (327.7±28.0 ms), 60 Hz (342.9±51.7 ms), and sham (333.0±30.0 ms). In the late phase, RT remained stable except for the sham group where we observed a reduction in RT: 120 Hz (327.9±28.6 ms), 60 Hz (337.9±56.9 ms), and sham (319.8±27.8 ms). The two-factor mixed ANOVA showed no statistically significant main effect of groups on RT (F=0.86, p=0.427). However, it showed statistically significant main effect of early/late adaptation on RT (F=6.62, p=0.013). A post hoc test revealed a statistically significant difference between early and late adaptation for sham (p<0.001), but no 120 Hz (p=0.959) nor 60 Hz (p=0.268). Also, this analysis showed that there was not an interaction between groups and early/late phase adaptation (F=2.81, p=0.068).

During the washout block, in the early phase, the reaction time per group was as follows: 120 Hz (330.7±24.4 ms), 60 Hz (338.7±61.9 ms), and sham (320.9±28.2 ms). During the late phase, RT remained approximately constant: 120 Hz (330.3±29.1 ms), 60 Hz (336.5±58.1 ms), and sham (317.8±30.6 ms). A two-factor mixed ANOVA showed no statistically significant main effects of groups (F=1.07, p=0.349) or early/late deadaptation (F=0.86, p=0.357). In addition, this analysis showed no interaction be-tween group and early/late deadaptation (F=0.141, p=0.868).

b. Peak velocities

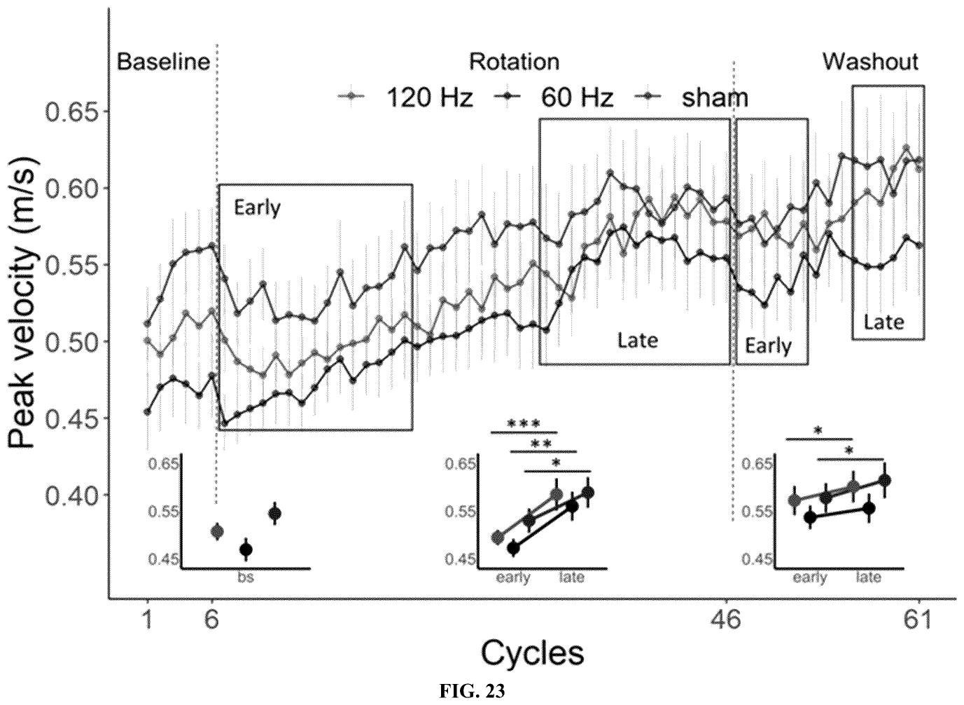

FIG. 23 shows the peak velocities per group during the experiment. During base-line, peak velocities were approximately 0.5 m/s (120 Hz: 0.51±0.086 m/s, 60 Hz: 0.47±0.11 m/s, sham: 0.54±0.11 m/s). A one-way ANOVA showed no statistically significant among groups (F=2.717, p=0.074).

During the rotation block, peak velocities in the early phase were distributed as follows: 120 Hz (0.49±0.07 m/s), 60 Hz (0.47±0.09 m/s), and sham (0.53±0.12 m/s). In the late phase, all groups experienced an increase in PV: 120 Hz (0.58±0.16 m/s), 60 Hz (0.56±0.14 m/s), and sham (0.59±0.15 m/s). A two-factor mixed ANOVA showed no statistically significant main effect of groups on PV (F=0.77, p=0.465). There was a statistically significant main effect of early/late adaptation on PV (F=34.04, p<0.001). A post hoc test revealed that there was a statistically significant between early and late phase of adaptation for all groups (120 Hz: p=0.001, 60 Hz: p=0.002, sham: p=0.017). Finally, the analysis showed that there was not a statistically significant interaction between groups and early/late adaptation (F=0.548, p=0.581).

Lastly, for the washout block, the specific PV per group was the following during the early phase: 120 Hz (0.57±0.14 m/s), 60 Hz (0.54±0.11 m/s), and sham (0.58±0.14 m/s). In the late phase, PV per group slightly increased: 120 Hz (0.60±0.15 m/s), 60 Hz (0.56±0.14 m/s), and sham (0.61±0.17 m/s). A two-factor mixed ANOVA showed no statistically significant main effect of groups on PV (F=0.73, p=0.488). In contrast, there was a statistically significant main effect of early/late phase deadaptation on PV F=13.24, p<0.001). A post hoc comparison revealed a statistically significant difference between early and late adaptation for 120 Hz (p=0.048) and sham (p=0.023) but no 60 Hz (p=0.111). Finally, the analysis showed no statistically significant interaction between groups and early/late deadaptation (F=0.43, p=0.65).

c. Directional error

All groups showed an average directional error close to zero during the baseline block: 120 Hz (−0.25°±1.1), 60 Hz (0.48°±1.57) and Sham (0.09°±1). A one-way ANOVA showed no statistically significant differences among groups (F=1.78, p=0.177).

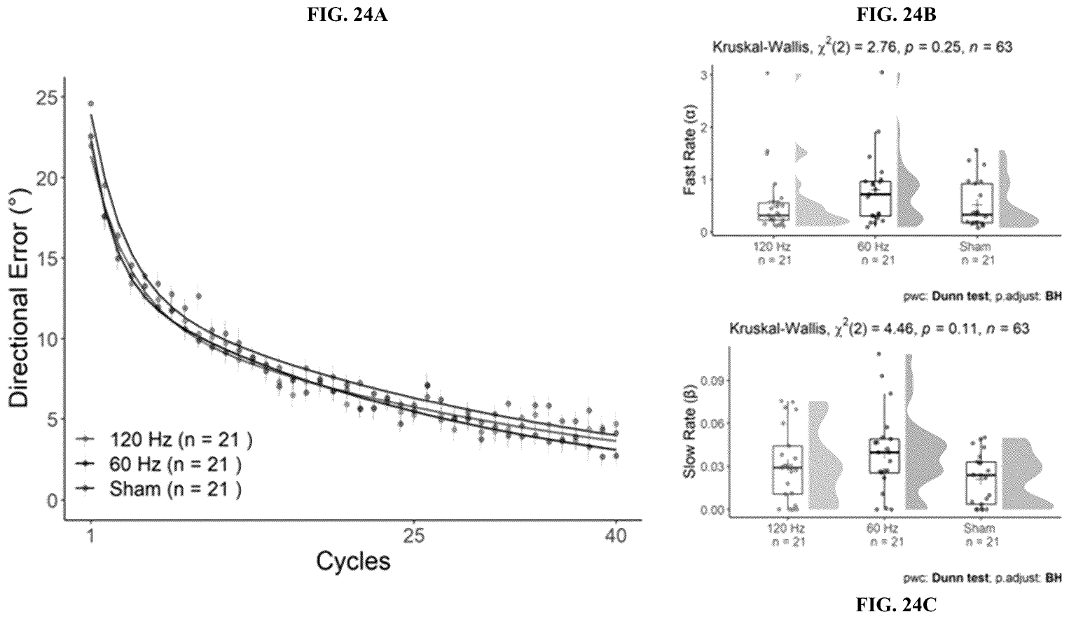

During the rotation block, learning curves in FIG. 24A shows that stimulation groups learned slightly faster than sham. However, as shown in FIGS. 24B and 24C, no statistically significant differences were found among groups for fast (Kruskal-Wallis test; χ2=2.76, df=2, p=0.25) and slow rates (Kruskal-Wallis test; χ2=4.46, df=2, p=0.11).

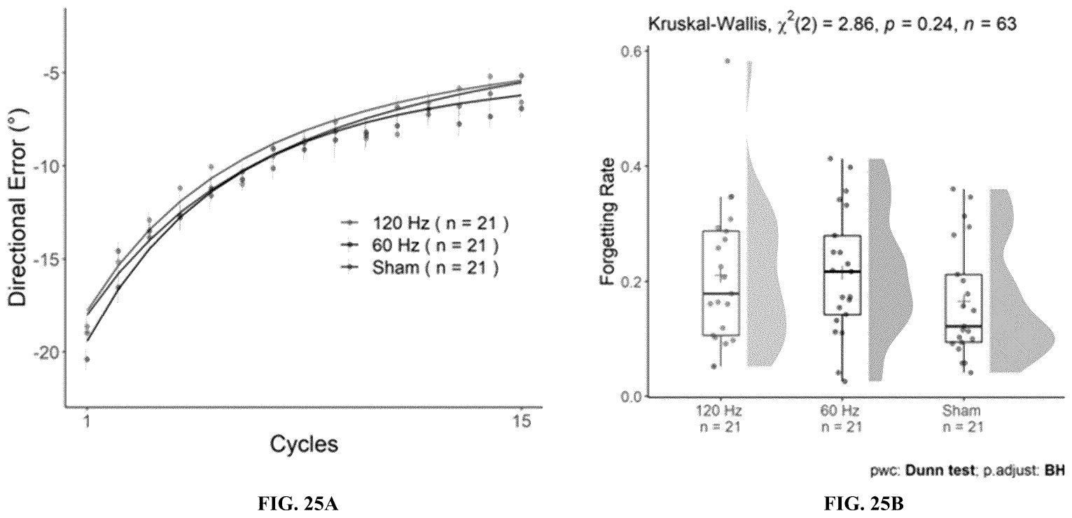

For the washout block, FIGS. 25A and 25B show that DEs followed the characteristic error pattern in the opposite direction but no difference in the rate at which performance returned to baseline levels between groups was found (Kruskal-Wallis test; χ2=2.86, df=2, p=0.24).

d. Post Study Survey

Out of sixty-one (61) subjects who completed the post-study survey, 14 individuals (23%) experienced discomfort or pain during the experiment. On average, they rated the discomfort at 3.4/10±1.4, indicating a mild level. Distributed by groups, two subjects in the 120 Hz group reported discomfort (rate=2.5/10±0.7), five in the 60 Hz group (rate=3.4/10±2.6), and seven in the sham group (rate=3.6/10±1).

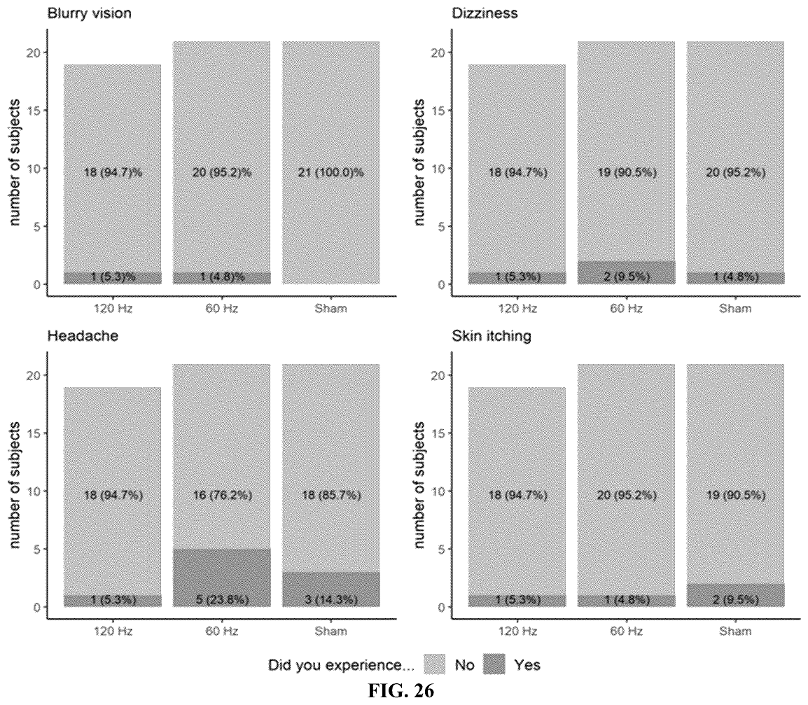

Additionally, consulted for headaches, dizziness, blurry vision, and skin itching, a small percentage of participants across all the groups reported having experienced at least one of them. For instance, only one participant in each active group reported blurred vision (120 Hz: n=1, rate=1/10; 60 Hz: n=1, rate=5/10). On the other hand, the 60 Hz group experienced 5 events (23.8%, rate=4.6/10±1.8) of headaches, followed by the sham group with 3 cases (14.3%, rate=5.3/10±3.5) and only one case in the 120 Hz group (5%, rate=2/10). Dizziness was reported in less than 10% of each group (120 Hz: n=1, rate=7/10; 60 Hz: n=2, rate=3/10±0; sham: n=1, rate=6/10), similar to skin itching at the electrode site (120 Hz: n=1, rate=3/10; 60 Hz: n=1, rate=4/10; sham: n=2, rate=5/10±4.2). FIG. 26 summarizes the side effects reported.

e. Sham Analysis

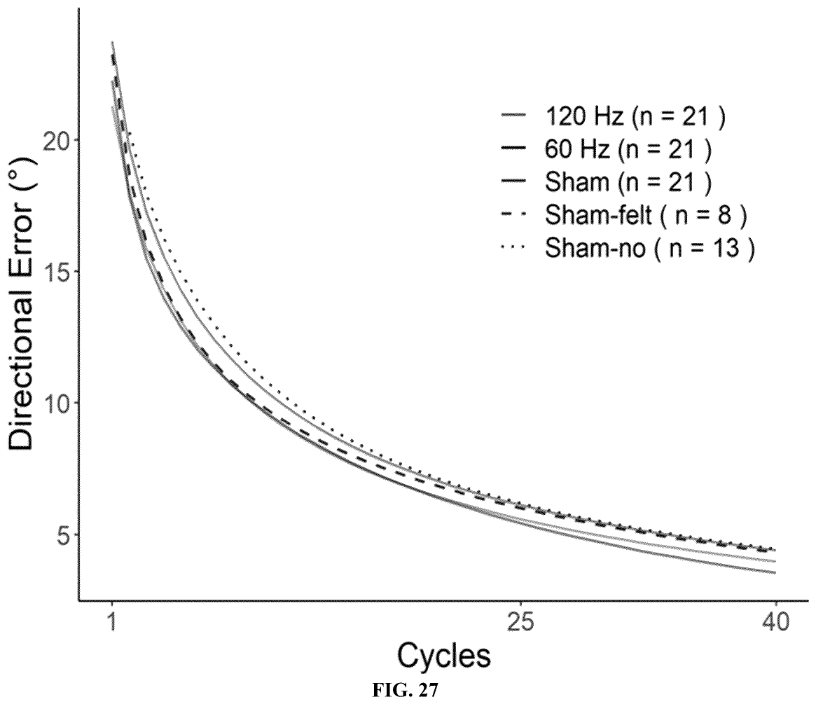

Similar to experiments 1A and 1B, 8 out of 21 subjects (38%) from the sham group reported to have perceived the stimulation even though no current was applied to them. FIG. 27 illustrates the learning curves for the sham group, divided into those who perceived TNS (Sham-felt; n=8) and those did not (Sham-no; n=13), as well as the active groups. It is possible to observe that the subgroup Sham-felt (blue dashed line) demonstrated a similar learning rate compared to the active groups, during the initial phase of learning. Then, it diverged tending to the sham overall performance. In contrast, the subgroup Sham-no (dotted blue-line) exhibited the slowest learning in the early phase of the adaptation but eventually converged to sham levels by the end of the rotation block.

f. Comparison Between Offline Vs Online 120 Hz and 60 Hz Studies

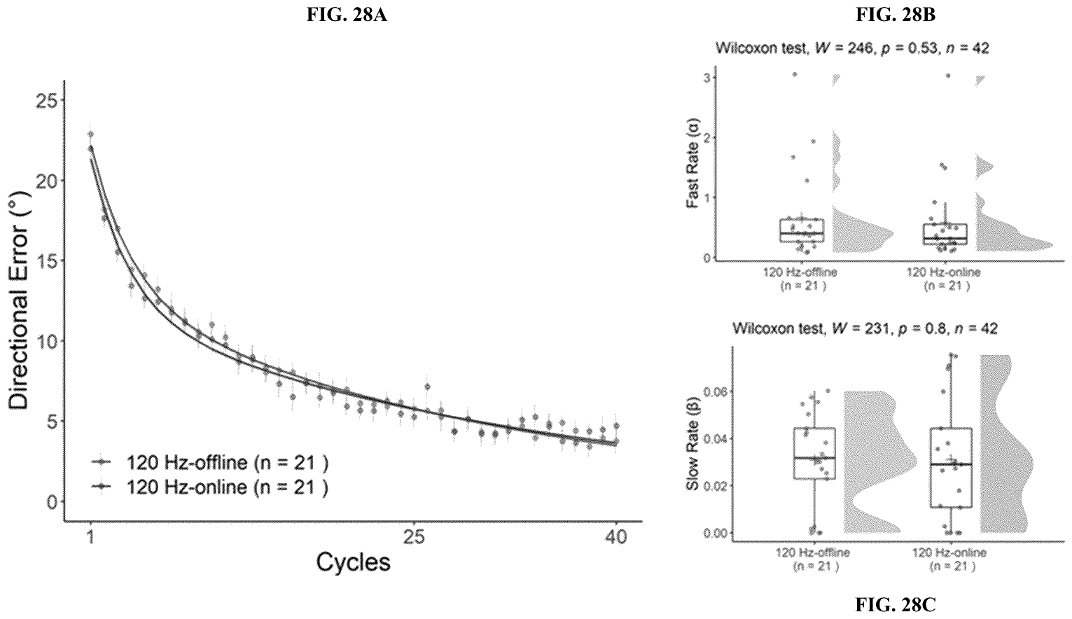

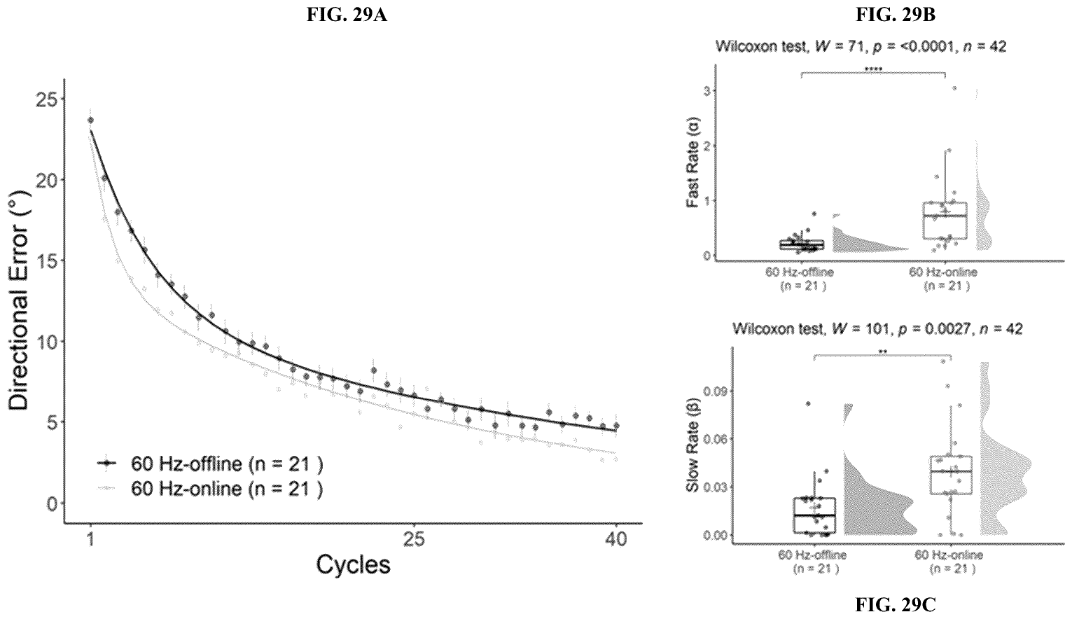

FIG. 28A shows the learning curves obtained from two different cohorts of subjects who received TNS at 120 Hz, applied online (maroon-bold line) and offline (red-bold line). As suggested by the overlapped learning curves, learning rates (FIGS. 28B and 28C) with online 120 Hz were similar to those previously observed with offline 120 Hz TNS (Wilcoxon test, α: W=246, p=0.53; β: W=231, p=0.8). On the other hand, learning curves showed in FIG. 29A suggests that 60 Hz online was markedly faster than those observed previously with 60 Hz-offline. This observation was confirmed by comparing the learning rates (Wilcoxon test, α: W=71, p<0.001; β: W=101, p=0.0027).

### 10. Discussion

a. Effects on Learning Rates and Comparison with Offline TNS

Experiment 2 explored the effects of 120 and 60 Hz TNS protocols delivered online on visuomotor learning. Given the results obtained in experiment 1A, which did not show learning improvement, the same 120 and 60 Hz protocols was assessed but applied TNS during task performance (online). More pronounced effects of TNS was expected, given the encouraging results observed using online stimulation in tACS, tDCS, and taVNS. However, even though learning curves from active groups showed slightly faster adaptation under perturbed conditions, learning rates analysis showed no statistically significant differences compared to sham.

Interestingly, when results from this experiment were compared directly with those observed in experiment 1A (offline TNS), significantly faster learning rates were observed for the online 60 Hz protocol. This result suggests that TNS-dependent effects on visuomotor adaptation depend on the timing of stimulation relative to task performance, at least for 60 Hz stimulation. In contrast, the online 120 Hz group maintained a similar learning rate as when this stimulation frequency was applied offline, showing no differences in learning between studies.

The online effects of transcranial stimulation have been attributed to the modulation of cortical excitability through the alteration of membrane properties by weak electric currents. On the other hand, TNS effects are thought to be produced by the activation of Locus Coeruleus (LC), subsequent release of Norepinephrine (NE) across the brain, and resulting modulation of synaptic plasticity. Because neurotransmitter release is a slow process, this mechanism might be more suitable for offline stimulation rather than online. However, the timing effects observed suggest another mechanism of action. Simulation studies have suggested that supraorbital TNS might also generate transcranial effects, activating frontal areas. Explicit learning has been associated with the activation of the frontal cortex, particularly with the PFC, a brain region involved in high-level cognition, decision-making and strategic learning. As depicted in FIG. 29, online TNS at 60 Hz showed relatively rapid learning compared to offline TNS, an effect that was more pronounced in the early adaptation phase. This suggests active involvement of explicit learning mechanisms, which are thought to be mediated by frontal areas.

b. Sham Groups Performance

In experiment 2, 38% of the sham group participants experienced sensations consistent with stimulation, similar to experiments 1A and 1B. It is worth noting that no electrical current was applied in any of these studies, despite the electrodes being placed on the forehead as in the active groups. Consistent with the observations in experiments 1A and 1B, individuals who reported feeling TNS initially showed rapid learning at a similar rate as active groups (blue dashed line, sham-felt). However, after approximately 15 cycles DEs deviated slightly, converging towards the levels in the sham-no group, which exhibited the slowest learning throughout the rotation block. This observation suggests that part of the sham might have been influenced by a placebo effect which could be masking stronger TNS effects.

c. Stimulation Tolerability

In line with findings in experiments 1A and 1B, 23% of all participants experienced some level of discomfort. Interestingly, the sham group reported the highest number of discomfort cases (7 cases). This observation suggests that the discomfort experienced may not be primarily caused by TNS. Instead, it likely comes from arm fatigue related to the repetitive nature of the reaching task. This issue is studied in Example 4.

Furthermore, participants were inquired specifically about headaches, blurry vision, skin itching, and dizziness. Headaches were reported by the highest number of participants with 5 (23.8%) in the 60 Hz group, followed by 3 (14.3%) in the sham group. These findings align with the results of experiment 1A, where 3 (14.3%) cases were reported in the 60 Hz group and 2 (10.5%) cases in the sham. In both cases, 120 Hz reported fewer than 10% of headache cases. While headaches have been associated with potential side effects in a small percentage of cases, it is noteworthy they were also reported in the sham group. This suggests that some of these headaches may be related to the experimental conditions rather than TNS itself, as discussed in Example 3. Factors such as the duration of the experiment, and prolonged screen exposure in VR settings can contribute to cybersickness which manifests as headaches and eyestrain, among other side effects. In fact, one participant discontinued the experiment due to eyestrain.

### 11. Conclusion

This study aimed to evaluate the effects of online TNS using clinically used frequencies on visuomotor learning. Although we observed a slight improvement in learning for the active groups, no statistically significant differences were found com-pared to sham.

Interestingly, in comparison to the findings from the offline study (experiment 1A), the 60 Frequency, which previously resulted in slow learning, demonstrated improved performance when delivered online. TNS effects are associated to the release of NE to the cortex by the LC, which could be a slow process, more suitable for offline stimulation. The unexpected outcome with online stimulation might suggests an alternative mechanism to exert its effects, related to transcranial effects and the ability to generate cortical excitability like tDCs. However, further investigation is required to fully understand and explore this possibility.

Although the 60 Hz protocol has shown the ability to modulate learning behavior and consequently, generate plasticity-related changes, it did not significantly accelerate learning compared to the sham group. As a result, the next chapter will focus on further investigating motor learning enhancement using a 3 kHz offline protocol, which showed the most promising results thus far.

### Example 4 Effects of TNS and ONS Delivered Independently and Concurrently on Visuomotor Learning

Combined Transcranial Direct Current Stimulation (tDCS) and Transcutaneous Auricular Vagus Nerve Stimulation (taVNS) applied during performance of a working memory task showed a tendency for larger improvements than expected based on results using these modalities individually, suggesting a potential synergistic effect. Similarly, another recent study that combined Trigeminal Nerve Stimulation (TNS) with Occipital Nerve Stimulation (ONS) showed strong therapeutic effects on migraine. Thus, TNS was combined with another modality to enhance the beneficial effects observed using the 3 kHz offline TNS protocol. ONS presents itself as a promising candidate for such joint stimulation, due to its convergence onto similar brainstem structures as the trigeminal nerve. More specifically, ONS has the potential to activate brain circuits responsible for endogenous neuromodulation of memory circuits via Locus Coeruleus (LC) mediated Norepinephrine (NE) release, similar to Vagus Nerve Stimulation (VNS) and TNS. Supporting this notion, a recent paper reported that ONS enhanced associative memory in in older adults. Furthermore, a new hypothesis suggests that the trigeminal and occipital nerve projections to the LC may play a role in the beneficial effects attributed to tDCS, which has been used in various contexts to study behavior and cognition, as well as in clinical settings for psychiatric disorders.

In this study, the feasibility of stimulating the trigeminal and occipital nerves both independently and simultaneously were examined and assessed to determine which neuromodulation approach provides the strongest effects on motor learning. To this end, the best performing protocol from previous experiments (1A, 1B and 2) was used (3 kHz offline) to assess the effects of each approach on a visuomotor learning task.

### 1. Participants

Eighty-eight (88) subjects participated in this single-blind sham-controlled study. All participants described themselves as right-handed. One subject was classified as mixed-handed according to the Edinburgh Handedness Inventory (score=50) but was also considered for the study. Four (4) subjects' data sets were discarded for the following reasons: technical issues (1 subject), participation in a previous TNS study from our lab (1 subject), data with more than 10% of movements removed during the data cleaning process (1 subject), and inability to complete the experiment due to lightheadedness (1 subject). In this last case, similar to the case reported in Example 2, the participant felt dizzy and lightheaded after less than 2 minutes of receiving stimulation. After his recovery, he commented that he has a fear of needles and always experienced the feeling of fainting during blood draw. This information was not disclosed during eligibility questionnaire questions and the consent form signing, even though these procedures specifically asked about history of vaso-vagal syncope and fainting episodes. He fully recovered after the stimulation stopped. The remaining eighty-four (84) subjects (18-34 y/o; 22.9±3.2 y/o; 27 female and 57 male) were randomly assigned one of 4 groups: TNS, ONS, TNS+ONS, and sham.

### 2. Apparatus and Behavioral Task

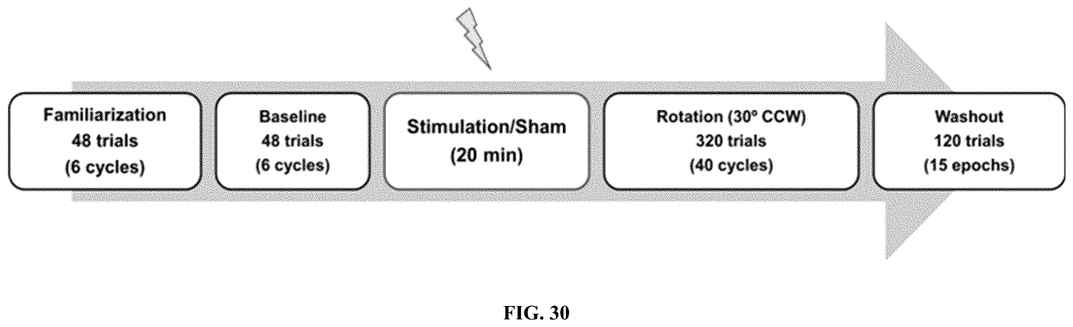

The same apparatus and behavioral task as described in Example 2 was used in this study. However, the experimental design of the visuomotor rotation task was modified by removing the post-stimulation baseline block used in previous offline TNS studies (experiments 1A and 1B), which reduced the time between the end of the stimulation session and the beginning of the rotation block. This was done to mitigate concerns that having the subjects perform an extra 5-minute baseline might jeopardize the window for potential neuromodulatory effects. As a result, participants experienced the rotation of their hand visual feedback immediately after receiving the stimulation, maximizing the probability that neuromodulation could influence motor adaptation. FIG. 30 shows the experimental design.

### 3. Stimulation Protocol

a. Active Groups Intervention

Similar to the experiments described in previous Examples, TNS was delivered by using two round 1.25 in (3.2 cm) diameter surface electrodes (Axelgaard Manufacturing Co., Ltd.) placed over the forehead, thereby targeting supraorbital branches of the trigeminal nerve. ONS was delivered using two round 1.5 in (3.81 cm) diameter carbon conductive rubber electrodes (Caputron Medical Products LLC.) combined with soaked-saline sponges. Rubber electrodes were placed 2 cm laterally from the occipital protuberance (Inion) targeting the bilateral branches of the greater occipital nerve and C2/C3 dermatomes. Each sponge was soaked with 6 ml of 0.9% sodium chloride solution (saline). Electrodes were replaced every 21 participants and sponges were used only once per subject.

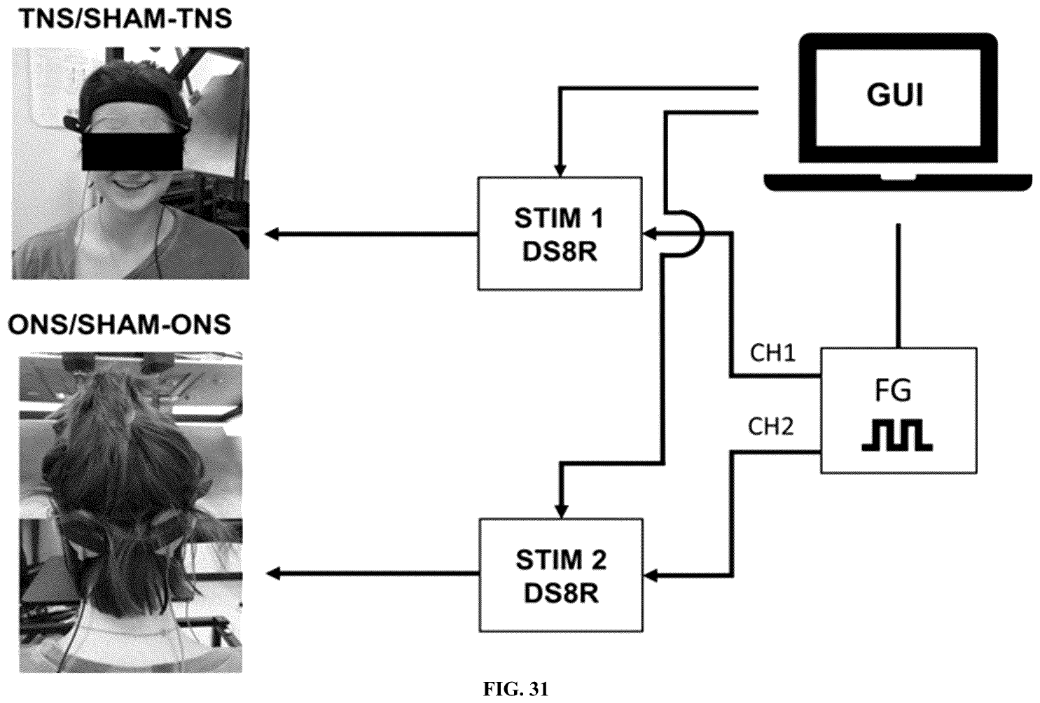

FIG. 31 shows a schematic diagram of the TNS/ONS setup up that allowed delivery of stimulation to the forehead and the back of the head either independently or concurrently. To this end, two (2) DS8R (Digitimer Ltd.) stimulators triggered by a dual-function generator (AFG 3022B, Tektronix Ltd.) with zero delay were used. All devices were connected to a laptop and controlled by a custom GUI written in MATLAB that allowed the experimenter to deliver either TNS, ONS or TNS+ONS with the same parameters used in experiment 1B. Thus, TNS and ONS were delivered at 3 kHz, using a pulse width of 50 μs, and an interphase interval of 1 μs for 20 minutes in cycles of 30 seconds ON and 30 seconds OFF. Similar to previous stimulation protocols, the current was ramped up/down over 5 seconds to make the ON/OFF transitions more comfortable.

TNS and ONS electrodes were placed on all participants. However, depending on the group assignment, either TNS, ONS, or both (TNS+ONS) were activated during the stimulation block. Despite this, all subjects, including those in the sham group, were informed the stimulation would be applied on the forehead and the back of the head concurrently using small currents, and because of that, they might or might not feel it. Also, they were told that a comfortable intensity threshold needed to be determined separately for each location before delivering concurrent stimulation. Finally, similar to previous studies, they were told that they should not feel any pain or discomfort and they could reduce the current anytime.

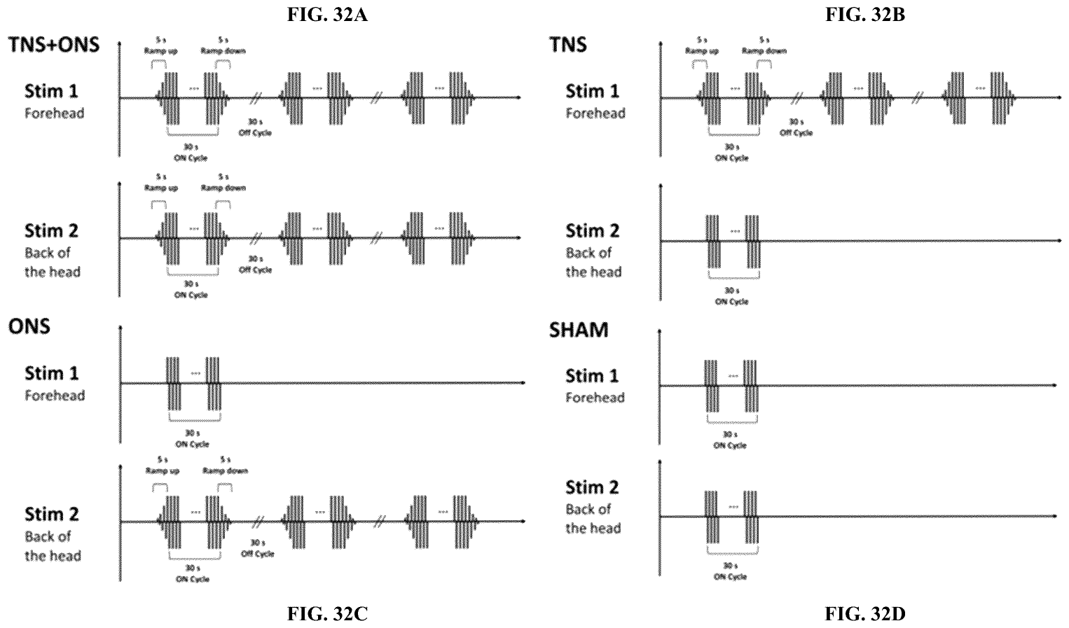

For concurrent stimulation (TNS+ONS group), the forehead and back of the head were stimulated with a submaximal tolerable current (see FIG. 32A). This required the experimenter to perform a threshold search for each location separately. Here the current increased as long as participants were comfortable, the participant requested to stop at a particular level, or the current reached the safety threshold. The threshold search began with ONS at 2 mA and increased in 1 mA steps until the perceptual threshold level or 7 mA was reached. Then, the increment changed to 0.1 mA, and the current increased until the submaximal tolerable threshold or 16 mA (safety threshold for ONS) was reached. After the intensity was found, ONS was turned off, and the same procedure was performed for TNS. In this case, the current went from 0 to the perceptual threshold in steps of 1 mA or 5 mA. Then, the increment also changed to 0.1 mA, and the intensity increased until the submaximal tolerable threshold or 13 mA (safety threshold for TNS) was reached. Finally, TNS and ONS were delivered concurrently using the current levels determined in the individual threshold searches. The experimenter assessed the participant's comfort, and currents were adjusted if necessary. Once the threshold search was completed, the stimulation was delivered for 20 min, and subjects were instructed to watch the same sequence of neutral images used in experiments 1A and 1B.

For independent stimulation (TNS and ONS groups), the individual threshold search was performed only for the active location. However, in order to follow the same protocol described above, the inactive location received sham stimulation. Then, both locations were stimulated concurrently during the first 30 seconds cycle, and then only the assigned stimulation location was active for the remainder of the stimulation block (see FIGS. 32B and 32C).

Once participants completed the stimulation block, the electrodes were removed and they were asked to indicate whether they felt the stimulation. If so, they sketched where they felt it using a diagram of the face and the back of the head anatomy. Following this, they immediately performed the rotation block.

b. Sham Intervention

Unlike experiments 1A, 1B, and 2, in which the sham group did not receive stimulation, this study used an active-sham approach to reinforce subject blindness. To achieve this, participants received 60 seconds of fixed current at each location, divided into two doses of equal duration. The first dose was administered during the threshold search, while the second dose was given during the initial 30-second cycle of the stimulation session. The currents used for sham-TNS and sham-ONS were 6 mA and 9 mA, respectively, resulting in approximately equivalent current densities (ONS: 0.789 mA/cm², TNS: 0.746 mA/cm²). These intensities attempted to evoke the perception of the stimulation using the lowest current possible. Other parameters such as frequency and pulse width were the same as those in the active groups. Thus, the experimenter delivered the fixed level for 30 seconds and then pretended to increase the current approximately up to one minute after the stimulation was turned off. If the subject did not request to stop at that time, the experimenter indicated that the safety threshold was reached. This procedure was performed first for sham-ONS and then for sham-TNS. If either one or both fixed currents produced discomfort, they were reduced to a well-tolerated level. Finally, both TNS and ONS were delivered concurrently for 30 seconds and then turned off for the remainder of the stimulation block (see FIG. 32D).

c. Post Study Survey

Following the standard protocol used in other studies disclosed herein, participants were requested to complete an online post-study survey upon the conclusion of the experiment. In conjunction with the inquiries outlined in Example 1, three additional questions were included to enhance the evaluation of previously identified trends discussed in earlier chapters. For instance, in experiments 1A, 1B, and 2, between 10% and 37% of participants reported experiencing pain or discomfort but it was uncertain whether the discomfort reported was produced by arm fatigue due to the multiple arm movements performed or the applied stimulation. Therefore, a question was added to determine the source of the discomfort. Consequently, participants were inquired where the discomfort was localized, providing alternatives such as the forehead, back of the head, or the right arm.

Furthermore, a question was added to evaluate the effectiveness of the sham intervention. In this regard, participants were asked if they felt they had received stimulation or not and, if so, where they thought the stimulation had been applied. Additionally, to gain deeper insights into possible placebo effects, individuals who did not receive stimulation but perceived that they did, were asked if they noticed any enhancement in their motor performance during the experiments.

### 4. Data Analysis

a. Analyses of Baseline Performance

After checking normality using the Shapiro-Wilk test, Directional Error (DE), Peak Velocity (PV) and Reaction Time (RT) comparison between groups during the baseline block were tested using a one-way ANOVA.

b. Analyses of Adaptation and Post-Adaptation Performance

DE, PV, and RT during the rotation and washout block were analyzed based on the methods described in Example 1. In addition, similar to experiment 1A, 1B, and 2, Kruskal-Wallis test was used to compare learning and forgetting rates because they were not normally distributed (Shapiro-Wilk test p>0.05).

### 5. Results

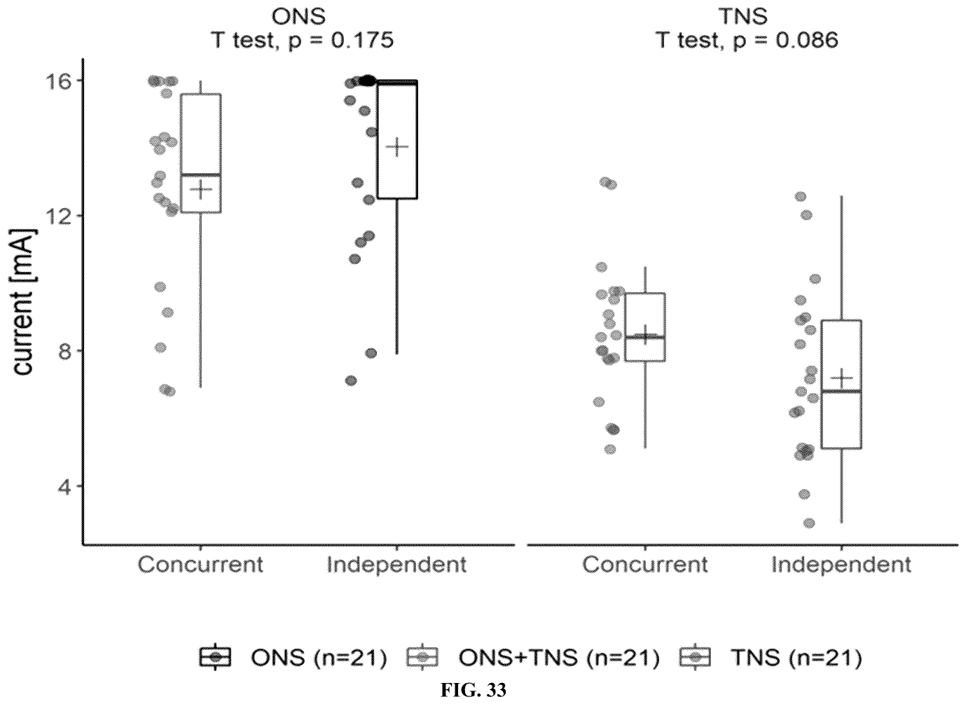

On average, participants who received TNS and ONS independently were able to tolerate 7.2±2.6 mA and 14.0±2.8 mA, respectively. On the other hand, subjects who received TNS and ONS concurrently received 8.5±2.1 mA (for TNS) and 12.8±3.0 mA (for ONS), on average. No significant differences were found between intensities levels delivered to the same location, depending on whether the stimulation was de-livered concurrently or independently (T-test, ONS: t(39.8)=−1.38, p=0.175; TNS: t(38.7)=1.76, p=0.086). FIG. 33 shows the distribution of intensities tolerated by participants per group.

a. Reaction Time

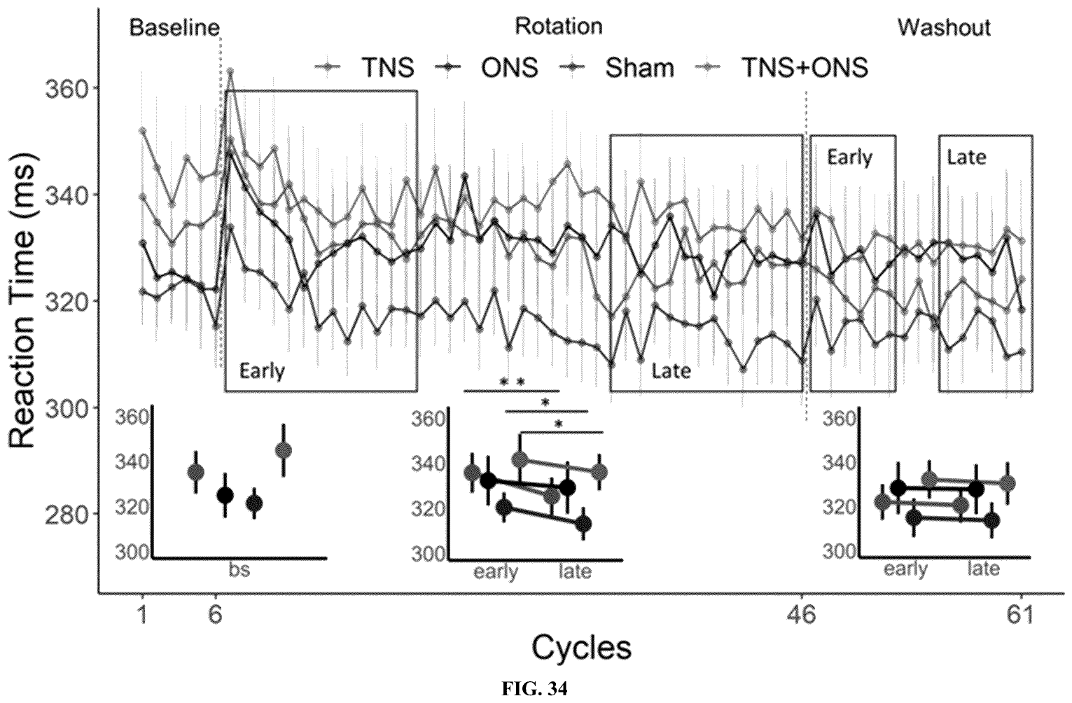

FIG. 34 shows the reaction time throughout the experiment for all groups. In the baseline, a one-way ANOVA revealed that there were no statistically significant differences in reaction time between groups (F=1.191, p=0.319). Specifically for each group, the mean RT was as follows: TNS (335.0±43.6 ms), ONS (324.9±45.8 ms), TNS+ONS (344.8±54.4 ms), and sham (321.3±32.0 ms).

In the early phase of the adaptation, the mean RT for each group was as follows: TNS (335.7±41.0 ms), ONS (332.5±50.6 ms), TNS+ONS (341.5±53.7 ms), and sham (320.3±30.8 ms). All groups experience a slight reduction of the reaction time in the late deadaptation: TNS (325.3±38.1 ms), ONS (329.1±53.6 ms), TNS+ONS (336.0±37.0 ms), and sham (313.1±33.9 ms). A two-factor mixed ANOVA showed no statistically significant effect of groups on RT (F=0.88, p=0.456). However, it showed a statistically significant main effect of early/late phase on RT (F=17.68, p<0.001). A post hoc pairwise comparison showed that there was a statistically significant difference between early and late adaptation for TNS (p=0.005), TNS+ONS (p=0.021) and sham (p=0.021) but not for ONS (p=0.457). Moreover, no statistically significant interaction between groups and early/late phase was found (F=0.796, p=0.5).

Finally, the reaction time reached a plateau during the washout block, the mean reaction time for the early adaptation phase for each group was as follows: TNS (322.0±37.1 ms), ONS (328.3±54.2 ms), TNS+ONS (332.1±40.0 ms), and sham (314.9±40.2 ms). In the last phase, the mean RT was the following: TNS (320.5±36.3 ms), ONS (327.7±51.9 ms), TNS+ONS (330.3±44.5 ms), and sham (313.7±37.5 ms). A two-factor mixed ANOVA showed no statistically significant main effect of groups (F=0.52, p=0.668) or (F=0.45, p=0.504) early/late phase on RT. Also, there was no interaction between groups and early/late deadaptation (F=0.014, p=0.998).

b. Peak Velocity

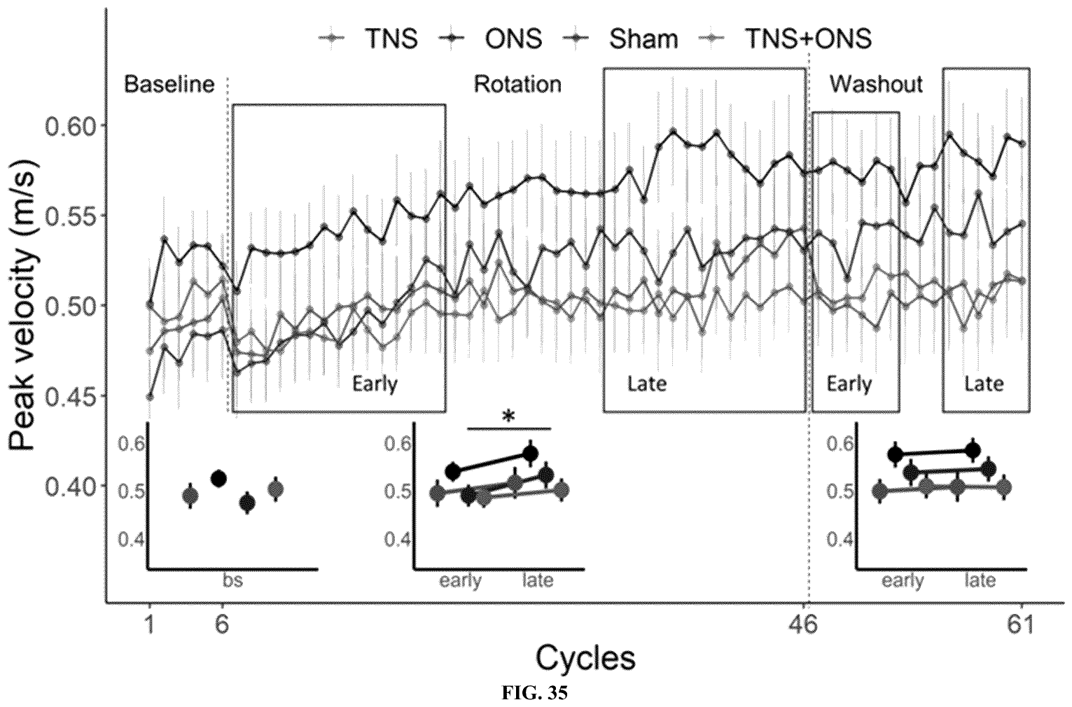

FIG. 35 shows the peak velocity data for all groups. The figure indicates that mean PV for all groups tended to increase across the experiment. During the baseline block, average PV for each group were the following: TNS (0.49±0.12 m/s), ONS (0.53±0.09 m/s), TNS+ONS (0.5±0.12 m/s), and sham (0.47±0.11 m/s). No significant differences were found between groups (F=0.786, p=0.505).

During the rotation block, all groups saw their peak velocity slightly increase as the experiment progressed. During the early phase of the adaptation, the mean PV for each group was as follows: TNS (0.49±0.13 m/s), ONS (0.54±0.10 m/s), TNS+ONS (0.49±0.10 m/s), and sham (0.49±0.11 m/s). For the late phase, the mean PV was as follows: TNS (0.52±0.15 m/s), ONS (0.58±0.13 m/s), TNS+ONS (0.50±0.11 m/s), and sham (0.53±0.13 m/s). A two-factor mixed ANOVA showed no statistically significant main effect of groups (F=1.30, p=0.28) on PV. However, there was a statistically significant effect of early/late phases (F=12.48, p<0.001). A post-hoc comparison revealed a statistically significant difference between early and late phases for sham (p=0.027) but not for ONS (p=0.094), TNS (p=0.07), and TNS+ONS (p=0.283). Additionally, the analysis also revealed that there was not a statistically significant interaction between groups and early/late adaptation phases (F=0.6, p=0.62).

During the washout, particularly in the early phase, the mean PV was as follows: TNS (0.50±0.12 m/s), ONS (0.58±0.13 m/s), TNS+ONS (0.51±0.12 m/s), and Sham (0.54±0.13 m/s). In the late phase, participants maintained the mean PV: TNS (0.51±0.14 m/s), ONS (0.58±0.12 m/s), TNS+ONS (0.51±0.12 m/s) and sham (0.55±0.12 m/s). A two-factor mixed ANOVA showed no statistically significant main effects of groups (F=1.75, p=0.163) or early/late phases (F=0.94, p=0.334) on PV. Also, this analysis showed no statistically significant interaction between groups and early/late deadaptation (F=0.601, p=0.62).

c. Directional Error

The mean directional error during baseline for all groups was −0.17±0.13° and the average for each group was the following: TNS (−0.09±0.97°), ONS (−0.037±1.42°), TNS+ONS (−0.053±1.3°) and sham (−0.48±1.4°). A one-way ANOVA showed no statistically significant differences among groups (F=0.575, p=0.633).

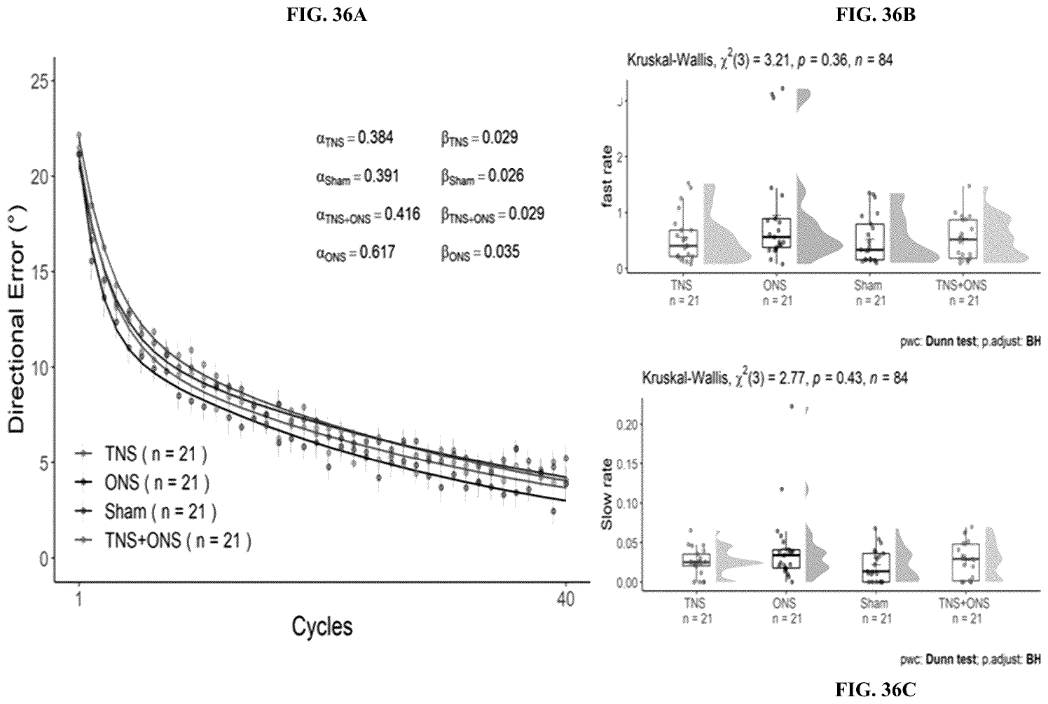

FIG. 36A illustrates the directional error across the rotation block and the respective learning curves for each group. ONS exhibited the fastest learning rates. The next group with the fastest learning curve was TNS+ONS, followed by the sham group. Interestingly, the TNS group demonstrated the slowest decay in the initial deadaptation phase and converging to sham level in the late phase. This observation contrast with the results observed in experiment 1B where TNS at 3 kHz showed faster learning compared to sham. Despite the observed trends in the learning curves, the analysis of learning rates (FIGS. 36B and 36C) revealed no statistically significant difference in fast (Kruskal-Wallis test; χ2=3.21, df=3, p=0.36) and slow learning rates (Kruskal-Wallis test; χ2=2.77, df=3, p=0.43).

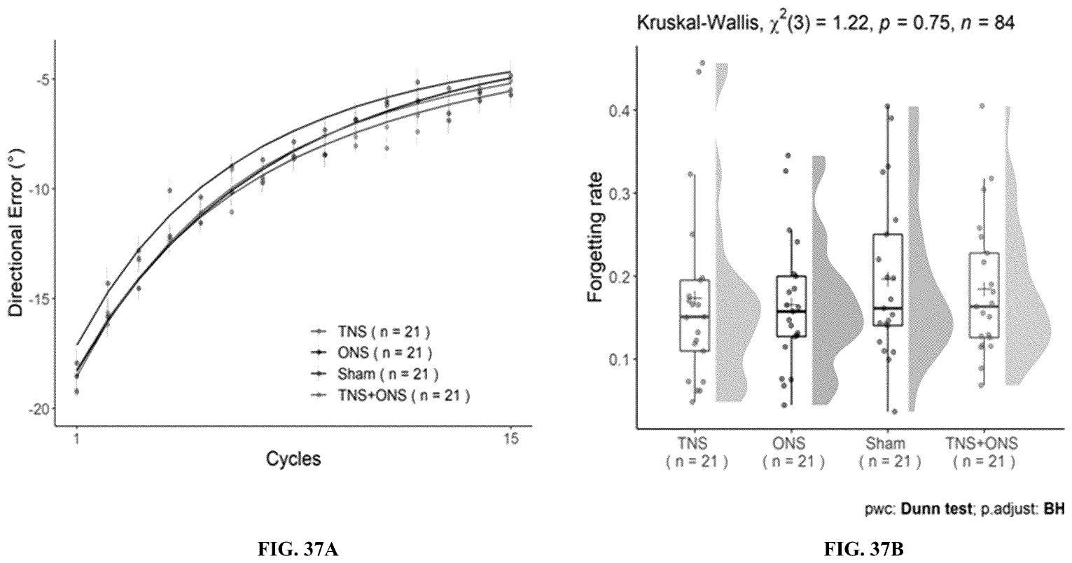

Finally, forgetting curves fitted to the DE data in the washout block are shown in FIG. 37A. The sham group tended to return to baseline levels faster than the active groups. However, rates analysis (FIG. 37B) did not reveal statistically significant differences among them (Kruskal-Wallis test; χ2=1.22, df=3, p=0.75).

d. Post Study Survey

The post-study survey was responded by 81 out of 84 participants, and 17.3% (14 out of 81) of participants reported experiencing discomfort or pain during the study. The level of discomfort or pain by the subjects were rated with 4.3±1.4 within a 1-10 scale. When consulted for the location of the discomfort, 78.6% (11 out of 14 subject) indicated the right arm and shoulder, 14.3% (2 out of 14 subject) the forehead, and 7.1% (1 out of 14 subject) reported both. The three participants who experienced discomfort in the forehead belonged to ONS+TNS (2 subjects) and ONS (1 subject). The ONS+TNS participants received 9.1 mA and 9.8 mA, and gave a discomfort level of 1 and 5, respectively. On the other hand, the ONS participant complained during the sham-TNS period. She immediately reported discomfort when the stimulation was on. Thus, the fixed threshold of 6 mA was reduced to 5.1 mA for the remainder of the sham-TNS (60 seconds), which was well-tolerated.

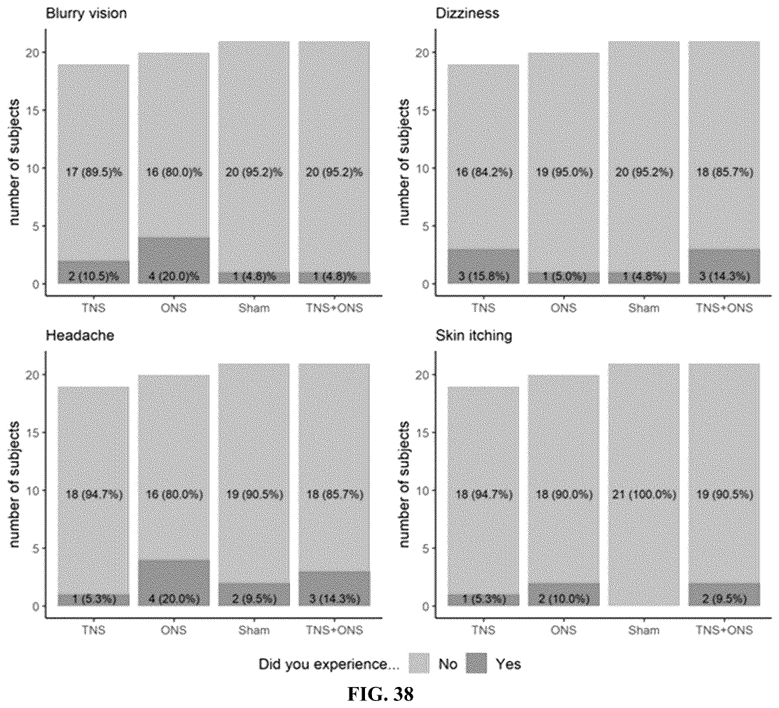

Additionally, subjects who belonged to the active groups reported tingling and pressure in the forehead and back of the head during the stimulation. Also, a small percentage of participants reported to have experienced a headache (TNS: n=1, rate=4/10; ONS: n=4, rate=5/10±2.4; TNS+ONS: n=3, rate=3.7/10±2.5; Sham: n=2, rate=3.5/10±0.7), dizziness (TNS: n=3, rate=4.7/10±0.6, ONS: n=1, rate=2/10; TNS+ONS: n=3, 4.3/10±3.2; Sham: n=1, rate=3/10), blurry vision (TNS: n=2, rate=3.5/10±0.7; ONS: n=4, rate=5.3/10±2.1; TNS+ONS: n=1, rate=1/10; Sham: n=1, rate=2/10) and skin irritation at the electrode site (TNS: n=1, rate=3/10; ONS: n=2, 4.5/10±0.7; TNS+ONS: n=2, rate=2/10±0; Sham: n=0) during or after the experiment. FIG. 38 summarize these results.

e. Sham Analysis

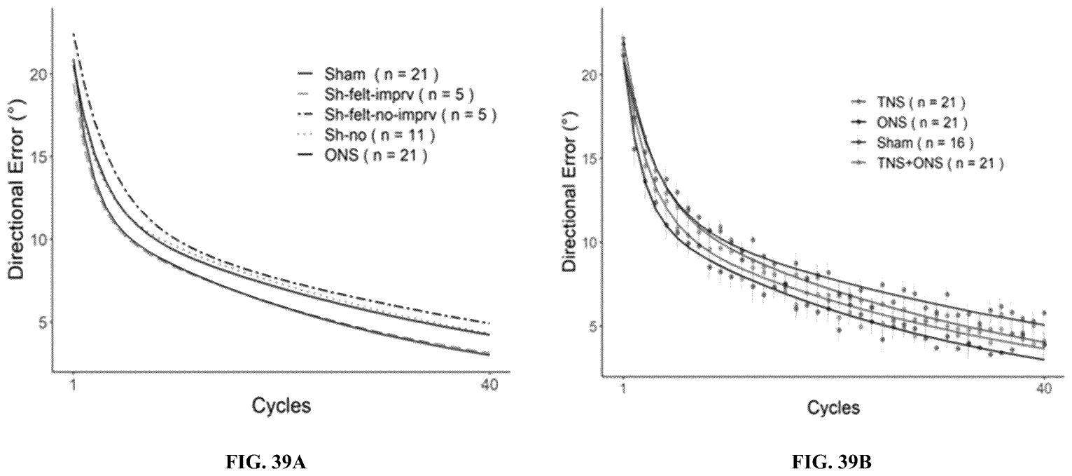

Within the sham group, 47.6% (10 out of 21) of the participants believed they received stimulation. The sham approach involved 60 seconds fixed current, which was different from experiments 1A, 1B, and 2, where no current was delivered. From this subgroup, 50.0% (5 out of 10) believed their motor performance improved after the stimulation. The learning curves for these subgroups of the sham group were examined to assess for potential placebo effects. The sham group was split into three subgroups: (1) participants who felt the stimulation and perceived motor improvement (Sh-felt-imprv, n=5), (2) participants who did not feel improvement despite feeling the simulation (Sh-felt-no-imprv, n=5), and (3) participants who did not feel the stimulation at all (Sh-no, n=11). FIG. 39A illustrates the learning curves for the sham and its subgroups, as well as the ONS group, which had the fastest improvement. It is possible to observe that individuals who experienced the stimulation and perceived improvement exhibited rapid learning at a similar rate to those in the ONS group. On the other hand, participants who did not perceive any improvement showed the slowest learning. Those who did not feel the stimulation showed a similar rate of learning than the overall sham group.

Considering that Sh-felt-imprv group's improvement can be attributed to a placebo effect, we excluded this subgroup from the sham group. Consequently, recalculating the learning curves (FIG. 39B) resulted in all the active groups outperforming the sham group.

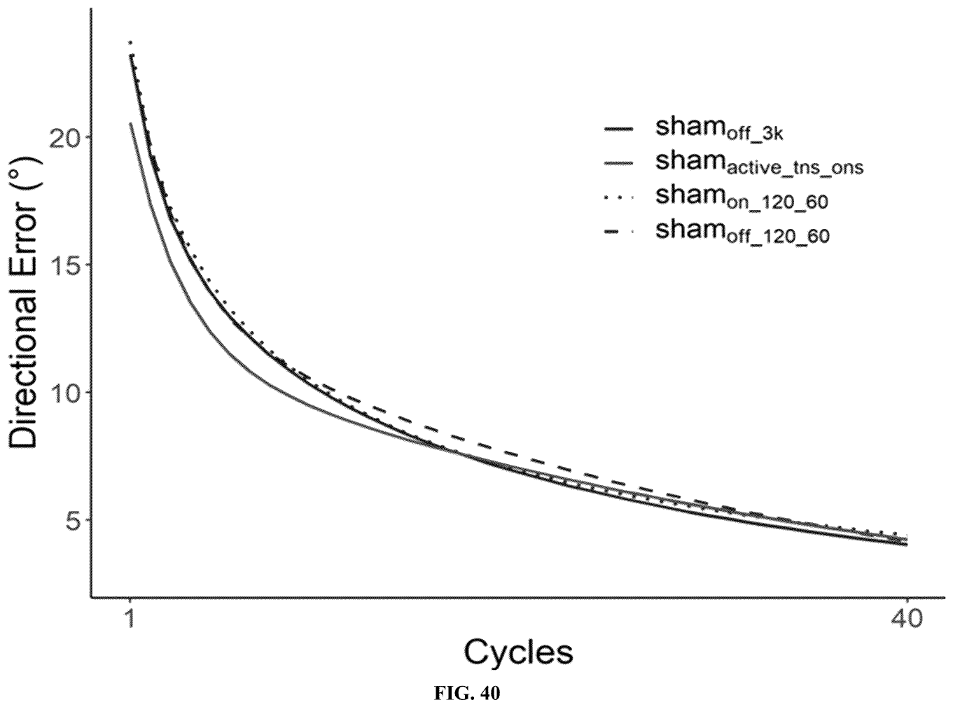

Furthermore, the active sham learning curve was compared with the passive sham from previous experiments. FIG. 40 shows that the learning curves for sham group in experiments 1A, 1B, and 2 (blue lines) overlap, suggesting a similar learning rate for all of them. On the other hand, the active sham group from experiment 3 adapted to the rotation faster than the passive sham during the early part of the adaptation but then converged with the passive sham in the late part of the adaptation. This comparison suggests a potentially stronger placebo effect in the active sham group than the passive sham group.

### 6. Discussion

a. Effects on Learning Rates

Analyses of learning curves revealed that the groups exposed to ONS demonstrated the fastest learning rates among groups. However, learning coefficients comparison did not confirm the effect. Surprisingly, the learning rates for TNS did not appear to be substantially faster than those of the sham group, in contrast to the results presented in Example 2, where it showed rapid learning rates compared to a passive sham (no current delivered). Moreover, subjects who received concurrent stimulation learned faster than the sham and TNS groups but slower than the ONS alone, suggesting that there are no synergistic effects using this protocol as initially hypothesized. These results suggest that ONS may have more beneficial effects on motor learning than TNS.

There are some factors that might explain the differences between modalities. For instance, the electrode size. ONS was delivered using soaked-saline sponges that allows stimulation through the hair as usually done in tDCS studies. These sponges had a larger round shape with a diameter of 3.81 cm, whereas the TNS electrodes had a diameter of 3.2 cm. This difference in size might have influenced the participant's ability to tolerate current. On average, the ONS group tolerated higher intensities (14.0±2.8 mA) than TNS (7.2±2.6 mA), resulting in higher current densities for ONS (1.228 mA/cm²) than TNS (0.895 mA/cm²). The higher amount of current and density delivered for ONS might have contributed to the observed effects.

Furthermore, larger electrodes can result in less focalized effects, potentially leading to the spread of current to other areas such as the cerebellum. It is well known the importance of the cerebellum in learning, and studies using cerebellar tDCS have shown affecting learning rates in a visuomotor learning task. While this study focused on the ability of ONS to activate bottom-up mechanisms, the possibility that the cerebellum was indirectly stimulated through a transcranial mechanism cannot be dismissed. In a published study, they investigate the effects of tDCS over the occipital nerve on memory improvement. To determine whether the observed effects were mediated by transcranial or transcutaneous mechanisms, they blocked the nerve using a skin anesthetic. The published study observed that the anesthetized group still show improvements compared to sham, although not to the same extent as without anesthesia. This suggests that some current might reach the brain and produce a cortical effect. In tDCS studies, the current is approximately 2 mA, significantly less than the average used in our study. Although, we used biphasic pulses with a 50 μs pulse width, instead of direct current, the use of higher intensities might still contribute to transcranial effects.

b. Sham Groups Might be Affected by Placebo Effects

Based on the findings from experiments 1A, 1B, and 2, it was consistently observed that subjects from the sham group who perceived the stimulation performed at a rate similar to the faster performers. This experiment was not the exception. Participants who reported feeling and perceiving motor improvement due to the stimulation learned the rotation at a similar rate compared to ONS. However, individuals who felt the stimulation but did not perceive any improvement presented impaired learning.

Thus, assuming the overall performance of the sham group was affected by a placebo effect, its learning curve was recalculated by excluding subjects who felt the stimulation and perceived motor improvement. When these participants were removed, all active groups, including TNS, performed faster than the sham. This approach allows controlling factors such as perception of receiving the stimulation and improvement of performance which might influence learning behavior.

In this experiment, an active approach was used, where participants received 60 seconds of stimulation to reinforce blinding which has been used in tDCS studies. Interestingly, when comparing the learning profile of the active sham group with the passive sham approach used in experiments 1A, 1B, and 2, it was observed that the active sham exhibited faster learning in the initial part of the adaptation compared to the passive sham. One possible explanation for this improvement in the active sham group could be attributed to a stronger placebo effect resulting from the combination of sensations produced by the brief stimulation and the more extensive setup used for this study. The experimental setup included the placement of electrodes on the forehead and at the back of the head, along with a rubber band to hold the electrodes in the back. Subjects described sensations of pulsation, wetness (due to the saline), and tightness due to the presence of the rubber band and electrodes in the back even before the stimulation began. The combination of these sensations, coupled with the 60-second stimulation may have intensified the overall placebo effect observed in the previous studies.

Alternatively, although it is unlikely, another possible explanation for the improvement observed is that the brief stimulation might have triggered some neurobiological changes that modulate learning. This is something that has been discussed in the tDCS field, where placebo effects have been reported. For instance, both sham and actual tDCS were able to modulate EEG parameters such as P3 associated with working memory, suggesting that sham-tDCS might also alter neural processes. Therefore, despite the small duration of 60 seconds compared to the active group, the potential effects of sham stimulation cannot be dismissed, and further research is necessary to assess their effects.

This series of observations suggest that perception of stimulation and improvement of performance might play a role in learning. Consequently, unmasking the true effects of stimulation interventions becomes a challenge. For this reason, it is crucial to control these intrinsic factors. This way, researchers can have more tools to identify the effects of stimulation.

c. Stimulation Tolerability

Inquiring the subjects about the location of the discomfort allowed us to determine whether the discomfort was produced by the stimulation or fatigue in the arm due to the repetitive task. In this experiment, 17.3% of all participants (14 out of 81) reported discomfort. From this subgroup, 78.6% of the cases were due to pain in the arm or shoulder. As suspected, the cause of discomfort during the experiment was associated with fatigue in the arm and shoulder due to the multiple reaching movements performed for almost two hours. However, three participants (21.4%) experienced discomfort in the forehead associated with TNS, which was resolved by reducing the stimulation intensity. It is important to note that participants selected a threshold that they considered comfortable and could reduce the current at any time. Thus, extrapolating these findings to experiments 1A, 1B, and 2, it was likely the reported discomfort was associated with arm fatigue.

In general, participants tolerated well the stimulation. There was only one case of lightheadedness after two minutes of TNS, similar to the case described in 1B, where 3 kHz TNS was also used. This subject also did not disclose their history of fainting episodes. Additionally, participants were consulted specifically for side effects such as blurry vision, dizziness, headaches, and skin itching. For ONS, the most commonly reported was headache (4 out of 20, 20%) and blurry vision (4 out of 20, 20%). TNS as well as TNS+ONS groups reported dizziness in three cases, equivalent to approximately 15% of participants of each group. The occurrence of these adverse events was similar to experiment 1B and in line with what has been reported in the VNS and TNS studies.

### 7. Conclusion

Pronounced learning with ONS during the rotation block of a visuomotor learning task was observed; however, no statistically significant differences were found. Concurrently applying ONS and TNS did not reveal any synergistic effects on learning. Despite this, we observed some interesting trends. For instance, the ONS group had rapid learning among groups. Factors that might have contributed to these effects were the higher intensities tolerated compared to TNS and potential transcranial effects that might have activated cerebellar regions, which is well known for their influence on learning mechanisms.

We also observed a potential placebo effect in our sham group when we analyzed a subgroup of subjects who reported having experienced the stimulation and perceived motor improvement. This subgroup presented a learning rate similar to the ONS, which was the fastest curve. Recalculating the learning curves without these participants it was shown that all the active groups were faster than the sham. Interestingly, when TNS was compared with the overall sham, it did not replicate the results shown in experiment 1B, but when compared with the recalculated sham, shows similar results as our previous experiment. Thus, finding better ways to control placebo effects which have been described as strong in studies that consider devices like in the brain stimulation field will help to clear the true effect of these stimulation protocols.

### Example 5 Effects of Online and Offline Trigeminal Nerve Stimulation on Visuomotor Learning

Although much is known about the clinical effects of trigeminal nerve stimulation (TNS), effects on sensorimotor and cognitive functions such as learning have received less attention, despite their potential impact on neurorehabilitation. One-hundred and thirty-two (132) right-handed individuals without prior history of neurological or psychiatric disorders participated in two experiments to assess the effects of single-session TNS on motor learning in healthy adults.

For Experiment A, 67 participants were recruited. Data sets from four participants were discarded: one participant withdrew in the middle of the experiment due to an unforeseen conflict in her schedule, while the other three participants were excluded due to noisy kinematic data on more than 10% of trials (See Data Analysis). Data from the remaining 63 participants (18-36 y/o; 22.75±4.6; 32 female and 31 men) were analyzed. For Experiment B, 65 participants were recruited. Data sets from two participants were discarded: one due to complaints about eye strain which resulted in abandonment of the experiment and the other due to a deviation from the protocol that was discovered post hoc. Thus, data from 63 participants (18-32 y/o; 23.2±3.9 y/o; 12 female and 51 male) were analyzed.

Written consent was obtained from all participants in accordance with the Declaration of Helsinki. All subjects declared themselves as right-handed, but their handedness was also assessed using the Edinburgh Handedness Inventory (short form). All subjects reported having normal or corrected-to-normal vision. Eligible individuals participated in only one experiment and the Arizona State University Institutional Review Board approved both experimental protocols.

Both studies were designed as single-blind, randomized, sham-controlled experiments. Block randomization was used to ensure equal sample size across groups using Sealed Enveloped. In both experiments, subjects were assigned to one of three different groups defined by the frequency of the stimulation: 120 Hz, 60 Hz, and sham.

### 1. Apparatus

Participants performed visually guided reaching movements within a semi-immersive 3D virtual reality (VR) environment. An active LED motion tracking system (Visualeyez VZ-3000; Phoenix Technologies, Inc.) was used to track arm movements. Visual images rendered in Vizard 3.0 (WorldViz Inc.) were displayed on a stereoscopic 3D monitor (Dimension Technologies Inc.) and projected onto a mirror that was embedded within a metal plate oriented at a 45° angle with respect to the monitor. During the experiment, participants were seated with their head positioned on a chin rest and with their eyes aligned with the center of the mirror. To prevent the arm from being directly viewed, movements were performed behind the metal plate, but visual feedback of the fingertip was provided to the participants in the form of a virtual cursor that was projected onto the mirror.

### 2. Behavioral Task

Participants performed an upper extremity visuomotor rotation task, which has been commonly used to investigate motor adaptation, a component of motor learning. This task engages processes involved in both low-level motor execution and high level of cognition and has previously been used to explore the effects of other neuromodulation techniques such as transcranial direct current stimulation (tDCS) and transcranial magnetic stimulation on motor learning. The task required participants to gradually adapt to an imposed discrepancy between hand motion and corresponding visual feedback. To this end, center-out reaching movements were made to eight targets located 10 cm away from the starting position and 45° apart within a vertical plane. Subjects were instructed to move as fast and as accurately as possible, and they received online visual feedback of their movements as well as terminal auditory feedback for correctly hitting the target. To ensure that participants used approximately the same range of movement velocities, they also received terminal visual feedback about their peak movement velocity and were encouraged to achieve PVs greater than or equal to 0.5 m/s. Feedback about movement accuracy was also provided using a point-based scoring system, which was designed to make the task more engaging. In addition, a message encouraging subjects to take a break was displayed every 25 trials to reduce the fatigue in the arm though subjects could also rest as needed.

Experiment A—Offline Stimulation: Participants began with a familiarization block, where they were instructed on how to perform the task with veridical visual feedback and no stimulation. This was followed by one experimental baseline block of 48 trials, also performed with veridical visual feedback and no stimulation (‘baseline 1’). After baseline 1, participants received either 120 Hz-TNS, 60 Hz-TNS or sham-TNS for 20 min under resting conditions. During this period, they viewed images of neutral valence and arousal obtained from the International Affective Picture System (IAPS; 131 images with mean valence scores: 5.17±0.4, mean arousal scores: 3.36±0.76). This was done in order to minimize variations in emotional states across participants. Immediately after the stimulation block, a second baseline block of 48 trials with veridical feedback and no stimulation was performed (‘baseline 2’) to determine whether the stimulation affected baseline motor performance. After baseline 2, a single rotation block of 320 trials involving a 30° counterclockwise (CCW) rotation of hand visual feedback performed without stimulation was performed. The perturbation was applied only in the vertical plane containing the targets even though participants were able to move freely within a 3D space. The experiment ended with a washout block of 120 trials, where the rotation was removed, and no stimulation was applied. The experimental design corresponds to that shown in FIG. 7.

Experiment B—Online Stimulation: Like Experiment A, Experiment B began with a familiarization block followed by a baseline block of 48 trials to assess participant performance. Following this, participants experienced a 30° CCW rotation of the hand visual feedback and concurrent TNS for 200 trials. After “the TNS+rotation” block was completed, participants performed another 120 trials with rotated feedback but no stimulation. The experiment ended with a washout block of 120 trials, where the rotation was removed, and no stimulation was applied. The experimental design corresponds to that shown in FIG. 21.

In both experiments, participants were given no prior knowledge regarding the introduction and removal of the rotated visual feedback and no instructions regarding strategies to counteract the rotation. Also, immediately after either receiving TNS or sham stimulation, subjects were asked to mark on a diagram of the face anatomy whether they had felt the stimulation and the location of any sensations perceived. Finally, once the experiment was completed, subjects filled out a survey to determine potential side effects regarding the stimulation as well as their perceived level of attention during the task.

### 3. Stimulation Protocol

TNS was applied using two, round, 3.2 cm diameter, surface electrodes (Axelgaard Manufacturing Co., Ltd.) placed on either side of the forehead to bilaterally stimulate the supraorbital branches of the trigeminal nerve. Before the electrodes' placement, the forehead was cleaned with an alcohol prep pad to reduce the impedance between the electrode and the skin. FIG. 4 shows the stimulation protocol. A biphasic symmetric square waveform was delivered using a peripheral nerve stimulator approved for human research (DS8R, Digitimer Ltd.). The stimulator was triggered using a function generator (AFG 3022B, Tektronix Ltd.), controlled by a custom graphical user interphase written in MATLAB (The MathWorks, Inc.) that also allowed setting stimulation parameters such as the frequency, current amplitude, and pulse width. TNS was delivered in cycles of 30 s ON and 30 s OFF. During the ON periods, the current was ramped up/down over 5 s to make the ON/OFF transitions more comfortable. Other parameters such as the pulse width (250 μs) and interphase interval (1 μs) were the same for both groups.

For Experiment A, TNS was delivered prior to task performance (offline) for 20 min. Therefore, due to the ON/OFF periods, participants received 10 min of effective TNS. On the other hand, for Experiment B, TNS was delivered online, i.e., concurrent with task performance, and the stimulation time varied depending on how long subjects took to perform the task. For this reason, TNS was delivered only during the first 200 trials (25 cycles) of the rotation block, which were completed in ˜20 min on average. This way, the stimulation doses received in both experiments were comparable.

Before the stimulation session began, participants were told that a comfortable level of stimulation would be selected and that they may or may not feel the stimulation. The experimenter then gradually increased the stimulation current until it reached a tolerable level that was greater than 0 mA but less than the established maximum safety level of 6 mA. Once the intensity level was determined, it was used throughout the experiment, but participants could still request to reduce or stop the stimulation at any time. The intensity setting procedure was also applied to sham participants even though no stimulation was provided. Thus, electrodes were placed in the same location as the experimental groups and the stimulator still generated the same sounds as when it was delivering current, which helped to reinforce the sham. In both experiments, immediately after the stimulation session was completed (either active or sham) subjects were asked whether they had felt the stimulation as well as the location of any sensations.

### 4. Data Analysis

Kinematic analyses were performed in MATLAB. Hand movements were sampled at 125 Hz, filtered using a low-pass 2nd order Butterworth filter with a cutoff frequency of 6.25 Hz, and differentiated to obtain hand movement velocities. Velocity profiles were used to obtain movement and behavioral parameters such as movement onset (MO), peak velocity (PV), and reaction time (RT). MO was estimated as the first point in time at which the movement exceeded 10% of the PV. RT was measured from the moment the visual target was presented until MO. To determine PV, RT, and MO, a semi-automatic script written in MATLAB was used. Each trial was reviewed, and if MO was incorrectly detected, it was adjusted manually. Additionally, if a velocity profile exhibited multiple peaks or the movement trajectory did not follow an initially straight path, the trial was deleted.

Visuomotor performance was quantified by the directional error (DE), defined as the angular difference between the hand position at PV and the target direction. Individual DEs during the rotation and washout blocks were corrected by removing any intrinsic directional biases observed during baseline blocks (M. F. Ghilardi, J. Gordon, and C. Ghez, “Learning a visuomotor transformation in a local area of work space produces directional biases in other areas,” Journal of Neurophysiology, vol. 73, no. 6, pp. 2535-2539, 1995). More specifically, the mean directional error for each target direction was first calculated during each baseline block. For Experiment A, data from baseline 1 (pre-stimulation) was used instead of baseline 2 (post-stimulation) to estimate the intrinsic bias, in case the latter was affected in some way by the stimulation. Subsequently, these biases were subtracted from the directional error on each trial of the rotation and washout blocks.

Cycles were defined as eight consecutive trials (one per target direction). Within the cycles, any trial DE, PV, or RT exceeding two standard deviations from the mean was deleted. Subsequent analyses were performed on the binned data from the remaining trials. Overall, datasets with more than 10% of the total movements deleted were not analyzed further.

Statistical analyses were performed in R (R Core Team, 2014). Following the convention of several previous studies, (participant's mean DEs during the rotation block were fitted to a double exponential model (see Eq. 1). This allowed for quantification of the fast and slow learning processes that drive motor adaptation. To this end, a nonlinear least squares procedure based on the Levenberg-Marquardt algorithm was used (nlsLM function in R) to fit the following model:

$$= {C}_{1} {e}^{- α i} + {C}_{2} {e}^{- β i} \qquad ( 1 )$$

where [symbol] is the estimated directional error during the rotation block, α and β represent the fast and slow learning rates, C₁ and C₂ are the magnitudes of each exponential and i is the i-th cycle during the rotation block. It is assumed that α and β>0, α>β and C₁ and C₂>0.

On the other hand, mean DEs during the washout block were fitted to a single exponential model:

$$= {Ae}^{- γ i} + C \qquad ( 2 )$$

where [symbol] is the estimated directional error during the washout block, γ represents the forgetting rate, A is the magnitude of the exponential, i is i-th cycle during the washout block and C is a constant. We assumed that A<0 (error in the opposite direction) and γ>0.

Analyses of baseline performance: For Experiment A, two-factor mixed ANOVAs were conducted to examine the effects of the stimulation groups (between-subjects factor: stim group) and pre/post stimulation (within-subjects factor: epoch) on motor performance (mean RT, PV, and DE across cycles between baselines 1 and 2). For Experiment B, one-way ANOVAs were conducted to compare RT, PV, and DE between stimulation groups during the baseline block. For both experiments, Shapiro-Wilk tests were performed prior to the ANOVA tests to confirm normality.

Analyses of adaptation and post-adaptation performance: In both experiments, learning rate coefficients were not normally distributed, as assessed by the Shapiro-Wilk test and via examination of QQ plots. As a result, to compare the learning and forgetting rates across different groups, the non-parametric Kruskal-Wallis test was used. Pairwise-multiple comparisons using Dunn's test were performed if significant results were found and p-values were adjusted using the Benjamin-Hochberg correction.

Changes in RT, PV, and DE were also compared between the early and late phases of the adaptation block. To estimate early adaptation performance, the average RT, PV, and DE across the first 15 cycles were calculated for each subject's data set. Similarly, to estimate late adaptation performance, the mean values across the last 15 cycles were computed. Comparisons were performed using two-factor mixed ANOVAs (between-subject factor: stim group; within-subject factor: epoch). A similar approach was used for the de-adaptation performance. The early phase of the de-adaptation was estimated using the average of the first six cycles, while the late phase was estimated based on the mean of the last six cycles. Two-factor mixed ANOVAs were used on the averaged data (between-subjects factor: stim group; within-subjects factor: epoch). If significant results were found, p-values were adjusted using the Bonferroni correction.

Finally, cross study comparisons between learning curves and learning rates obtained in Experiments A and B were performed using the non-parametric Wilcoxon rank-sum test.

### 5. Results

a. Experiment A: Offline Stimulation

Average stimulation intensities were similar between active stimulation groups but varied somewhat between male and female participants. On average, participants from the 120 Hz and 60 Hz groups received 3.1±0.7 mA and 3.4±0.8 mA of TNS, respectively. No statistically significant differences in tolerated stimulation intensities between groups were found (t-test, t=−1.24, df=39.68, p=0.22). Average intensities for male participants (n=18, 3.7±0.7 mA) were generally higher than those of female participants (n=24, 2.9±0.7 mA; t-test, t=−3.6, df=37.7, p<0.001). Since effects of intensity on learning rates or reported attention levels were generally equivocal (see Other Effects of TNS), these differences were not examined further.

i. Directional Error (DE)

DEs were generally small during the baseline blocks and did not appear to be influenced by TNS delivery. During baseline 1, DEs were close to 0° for all groups (120 Hz: 0.1°±1.3, 60 Hz: −0.09°±1.3, Sham: −0.1°±1.3) and during baseline 2, DEs were also negligible (120 Hz: 0.3°±1.6, 60 Hz: −0.09°±1.6, Sham: −0.1°±1.3). A two-factor mixed ANOVA was used to assess the effects of group and pre/post-stimulation on baseline DEs. This analysis showed no statistically significant effects of group (F=0.36, p=0.696) or pre/post stimulation (F=0.12, p=0.732) on DE. Also, no statistically significant interaction between group and epoch (pre/post-stimulation) was found (F=0.36, p=0.697).

Analyses of DEs showed evidence of frequency dependent effects of TNS during the rotation block. DEs for all groups throughout the rotation block are illustrated in the form of learning curves. As expected, DEs decayed exponentially from approximately 25° at the beginning of the block, (which was close to the perturbation angle of 30°) to nearly 0° at the end, indicating that participants gradually adapted to the rotated environment. A two-factor mixed ANOVA conducted on the DEs obtained during the early and late phases of this block confirmed this decay (Table 1) but failed to capture the more subtle differences that could be observed between groups. As a result, we also analyzed the learning rate coefficients that were obtained by fitting the DEs from the entire block to double exponential models. Visual inspection of these curves suggests that the 60 Hz group exhibited a slower rate of adaptation than the sham and 120 Hz groups during the early adaptation phase (cycles 1-15). On the other hand, learning curves were initially similar between the 120 Hz and sham groups but gradually diverged, with the 120 Hz group exhibiting slightly smaller DEs than the sham and 60 Hz groups in the late adaptation block (cycles 35-40). Analysis of the corresponding learning rates revealed significant differences among groups for both fast and slow rates (Kruskal-Wallis test; α: χ2=14.62, df=2, p<0.001; p: χ2=10.32, df=2, p<0.001). Post hoc pairwise comparisons using the Dunn test revealed that the fast and slow rates from the 60 Hz group were significantly different from those of the 120 Hz (α: p<0.001, β: p<0.001) and sham groups (α: p<0.001, β: p<0.001). However, the fast and slow rates comparison showed no statistically significant differences between 120 Hz and Sham (α: p=0.705, β: p=0.906).

No frequency dependent effects of TNS were found during the washout block. During this block, all groups experienced aftereffects, i.e., DEs in the opposite direction that decayed with time, and which is thought to reflect a ‘forgetting’ of the perturbation. As in the rotation block, a two-factor mixed ANOVA conducted on the DEs obtained during the early and late phases confirmed this decay (Table 1). However, learning rate coefficients were highly variable across subjects during this block and as a result the rates at which the DEs decayed did not vary across groups (Kruskal-Wallis test, χ2=4.55, df=2, p=0.1).

TABLE 1
Results of two-factor mixed ANOVAs for RT, PV, and DE in Experiment A

| | | | Reaction | | Peak | | Directional | |
|---|---|---|---|---|---|---|---|---|
| | | | Time (RT) | | Velocity (PV) | | Error (DE) | |
| | | df | F | p | F | p | F | p |
| Baselines | Groups | (2, 60) | 0.21 | 0.810 | 0.80 | 0.452 | 0.36 | 0.696 |
| (b1 and b2) | pre/post | (1, 60) | 3.82 | 0.055 | 21.99 | p < 0.001 | 0.12 | 0.732 |
| | Interaction | (2, 60) | 0.36 | 0.701 | 1.59 | 0.213 | 0.36 | 0.697 |
| Rotation | Groups | (2, 60) | 0.61 | 0.545 | 0.78 | 0.463 | 0.65 | 0.524 |
| | Early/late | (1, 60) | 3.41 | 0.070 | 39.57 | p < 0.001 | 1365.86 | p < 0.001 |
| | Interaction | (2, 60) | 0.34 | 0.716 | 0.92 | 0.405 | 0.99 | 0.378 |
| Washout | Groups | (2, 60) | 0.85 | 0.432 | 1.15 | 0.322 | 0.54 | 0.583 |
| | Early/late | (1, 60) | 0.27 | 0.602 | 13.54 | p < 0.001 | 727.96 | p < 0.001 |
| | Interaction | (2, 60) | 0.75 | 0.476 | 0.02 | 0.981 | 0.92 | 0.402 |

ii. Reaction Time (RT) and Peak Velocity (PV)

Statistical analyses did not point to strong effects of offline TNS on RT and PV. Table 1 reports the results from two-factor mixed ANOVAs conducted on the mean RTs and PVs obtained in the different analysis epochs. Mean RTs for all groups fluctuated between 320 and 360 ms and did not appear to increase or decrease within or across blocks. This was confirmed by the ANOVA which showed no statistically significant main effects of stimulation group or epoch on RT in the baseline (pre/post), rotation (early/late), or washout (early/late) blocks. In addition, no statistically significant interaction effects between group and epoch were found.

Mean PVs for all groups fluctuated between 0.45 and 0.6 m/s throughout the experiment. Despite the absence of clear differences between groups, there was a notable trend within each group, showing a progressive increase from the beginning to the end of each block. For instance, PV increased significantly from the pre- to post-stimulation baseline (F=21.99, p<0.001), with post hoc comparisons indicating this effect was attributed to both the 60 Hz (p=0.005) and sham (p=0.005) groups. During the early adaptation phase, all groups experienced a reduction in PV compared to baseline levels, which coincided with the introduction of the perturbation. However, PV increased again towards the latter phase of the adaptation (F=39.57, p<0.001). Post hoc multiple comparisons identified significant differences within all groups between early and late adaptation (120 Hz: p=0.006; 60 Hz: p<0.001; sham: p=0.024). Finally, during the washout block, there was a significant increase in PV from the early phase to the late phase (F=13.54, p<0.001). Pairwise comparisons indicated that this effect was pronounced within the 60 Hz (p=0.038) and sham (p=0.025) groups. Here again however, no statistically significant interactions between stimulation groups and epoch were found. The absence of significance interaction effects between groups and epoch on RT and PV suggests that active TNS had little to no effect on these behavioral and kinematic variables.

b. Experiment B: Online Stimulation

In Experiment B, average stimulation intensities were similar between active stimulation groups and between male and female participants. Participants in the stimulation groups self-selected similar current levels (60 Hz: 3.3±0.8 mA; 120 Hz: 3.1±0.8 mA). A t-test showed that there was no statistically significant difference between these currents (t=−0.67, df=40, p=0.51). In contrast to Experiment A however, there was no statistically significant difference (t-test, t=−0.35, df=16.45, p=0.73) in the intensity levels tolerated by male subjects (n=34, 3.2±0.8 mA) and female subjects (n=8, 3.1±0.5 mA).

In contrast to Experiment A, where the stimulation time was fixed at 20 min, in Experiment B, the stimulation time depended on how long participants took to complete the first 25 cycles. Despite that, the average duration of the stimulation sessions was approximately 20 min (120 Hz: 21±4.2 min, 60 Hz: 22±4.1 min, sham: 21.8±3 min), similar to Experiment A. This similarity in stimulation duration facilitated comparisons between the two experiments, as described below.

i. Directional Error (DE)

In contrast to offline TNS, no evidence of frequency dependent effects of online TNS on DE was found. All groups showed average DEs that were close to zero during the baseline block (120 Hz: −0.25°±1.1; 60 Hz: 0.480±1.57; Sham: 0.09°±1) and a one-way ANOVA showed no statistically significant differences in baseline DEs among groups during this block (F=1.78, p=0.177). DEs decreased dramatically between the early and late phases of the rotation block, a difference that was confirmed statistically (Table 2). During the rotation block, learning rates for the stimulation groups appeared to be slightly faster than those of the sham group. However, no statistically significant differences were found among groups for either the fast rate (Kruskal-Wallis test; χ2=2.76, df=2, p=0.25) or slow rate coefficients (Kruskal-Wallis test; χ2=4.46, df=2, p=0.11). For the washout block, DEs followed the characteristic reduction in DEs over blocks but no difference in the rates at which performance returned to baseline levels among groups was found (Kruskal-Wallis test; χ2=2.86, df=2, p=0.24).

TABLE 2
Results of one-way (baseline block) and two-factor mixed ANOVAs
(rotation and washout blocks) for RT, PV, and DE in Experiment B

| | | | Reaction | | Peak | | Directional | |
|---|---|---|---|---|---|---|---|---|
| | | | Time (RT) | | Velocity (PV) | | Error (DE) | |
| | | df | F | p | F | p | F | p |
| Baseline | Groups | (2, 60) | 1.17 | 0.316 | 2.72 | 0.074 | 1.31 | 0.277 |
| Rotation | Groups | (2, 60) | 0.86 | 0.427 | 0.77 | 0.465 | 1.57 | 0.217 |
| | Early/late | (1, 60) | 6.62 | 0.013 | 34.04 | p < 0.001 | 684.78 | p < 0.001 |
| | Interaction | (2, 60) | 2.81 | 0.068 | 0.55 | 0.581 | 0.76 | 0.473 |
| Washout | Groups | (2, 60) | 1.07 | 0.349 | 0.73 | 0.488 | 0.93 | 0.399 |
| | Early/late | (1, 60) | 0.86 | 0.357 | 13.24 | p < 0.001 | 570.34 | p < 0.001 |
| | Interaction | (2, 60) | 0.14 | 0.868 | 0.43 | 0.653 | 0.37 | 0.695 |

ii. Reaction Time (RT) and Peak Velocity (PV)

Statistical analyses did not reveal strong effects of online TNS on RT and PV. Table 2 reports the results of these analyses. A one-way ANOVA was used to compare RTs and PVs across groups during the baseline block and a two-factor mixed ANOVA was used to analyze performance during the rotation and washout blocks. Mean RTs fluctuated between 320 and 360 ms, similar to Experiment A. During the rotation block, a significant reduction in RT was noted as the experiment progressed, reflected by a significant main effect of epoch (F=6.62, p=0.013). Post hoc multiple comparisons attributed this effect to the sham group (p<0.001). No other statistically significant main effects of stimulation group or epoch, nor any significant interaction effects were found for any of the blocks.

Mean PVs fluctuated between 0.45 to 0.65 m/s. No statistically significant main effects of stimulation group were found during baseline. During the rotation block, all groups increased their PV as the study progressed. A statistically significant main effect of epoch (early/late) on PV was found for this block (F=34.04, p<0.001) and post hoc testing revealed that the effect was evident in all groups (120 Hz: p=0.001; 60 Hz: p=0.002; sham: p=0.017). During the washout, there was also a statistically significant main effect of epoch (F=13.24, p<0.001) with post hoc comparisons indicating statistically significant differences between the early and late phases for the 120 Hz (p=0.048) and sham groups (p=0.023) but not for the 60 Hz group (p=0.111). Again, similar to Experiment A, the absence of significant interaction effects in this experiment suggests that online TNS had little to no effect on PV and RT.

c. Cross Study Comparison

A post-hoc direct comparison of DEs obtained with offline and online TNS emphasized the importance of stimulation timing on performance. Learning rates with online 120 Hz were similar to those observed with offline 120 Hz TNS, a result that was confirmed statistically (Wilcoxon rank-sum test, α: W=246, p=0.53; β: W=231, p=0.8). On the other hand, the learning curves suggest that 60 Hz online TNS resulted in markedly faster learning than what was observed with 60 Hz-offline TNS. This observation was confirmed by a statistical analysis that directly compared the learning rates (Wilcoxon rank-sum test: α: W=71, p<0.001; β: W=101, p=0.0027).

d. Sham Condition

Interestingly, sham subjects appeared to perform differently depending on whether they believed they had received stimulation or not. In Experiment A, five out of 21 subjects in the sham group reported having perceived the stimulation even though no current was applied to participants in this group. In Experiment B, this occurred in eight out of 21 subjects. As a result, the perception of received stimulation affected learning rates.

To this end, the sham group from each experiment was split into subjects who perceived the stimulation (‘Sham-felt’) and those who did not (‘Sham-no’). Interestingly, in Experiment A, the Sham-felt subjects learned the rotation at a similar rate as those receiving 120 Hz TNS, which trended toward faster learning. On the contrary, the Sham-no subjects initially performed in a manner similar to the 120 Hz subjects but later converged toward learning rates consistent with 60 Hz TNS, which was associated with the slowest learning rates. A similar behavior was observed in Experiment B where the Sham-felt subgroup (n=8) demonstrated a similar learning rate compared to the active groups during the initial phase of learning but later converged toward the overall sham performance. In contrast, the Sham-no subgroup (n=13) exhibited the slowest learning in the early phase of the adaptation but eventually converged to rates consistent with the overall sham levels by the end of the rotation block. The small numbers of subjects in each subgroup precluded statistical analyses of differences in learning rates between them. Nevertheless, these observations could have important implications for TNS applications and neuromodulation in general, as discussed below.

e. Other Effects of TNS

In Experiments A and B, 60/63 (˜95%) and 61/63 (˜97%) participants respectively completed the post-study survey. Table 3 summarizes these results. In both experiments, average levels of reported attention were higher than 7.8/10 across all groups, indicating that participants considered themselves to be engaged in the task. No statistically significant differences in reported attention levels among groups were found in either experiment (Kruskal-Wallis test, Exp A: χ2=0.096, df=2, p<0.953; Exp 2: χ2=0.253, df=2, p<0.881). Nevertheless, potential correlations between reported attention levels and the two learning rates was still explored for each group. In Experiment A, the 120 Hz group showed a moderately strong, positive correlation between attention level and the α coefficient (Pearson's r=0.48, p=0.032). However, no other statistically significant correlations between reported attention level and learning rates were found in either experiment. In addition, potential correlations between reported attention levels and stimulation intensities was also explored. In Experiment A, the 60 Hz group showed a moderate, positive correlation between reported attention level and tolerated current intensity (Pearson's r=0.53 p=0.014). No other statistically significant correlations were found in either experiment.

TABLE 3
Post-study survey results for Experiment A (Exp 1) and Experiment
B (Exp 2). Mean scores are reported on a scale of 1-10. For headaches,
blurry vision, dizziness, itching and discomfort, 1 represents
barely noticeable discomfort, and 10 represents unbearable pain.
For the attention level, 1 represents not paying attention, and
10 indicates being engaged and anticipating stimuli.

| | | 120 Hz | | | 60 Hz | | | Sham | | |
|---|---|---|---|---|---|---|---|---|---|---|
| | | mean | SD | n | mean | SD | n | mean | SD | n |
| Attention | Exp 1 | 7.8 | 2.2 | 20 | 8 | 1.8 | 21 | 7.8 | 2.3 | 19 |
| Level | Exp 2 | 8.1 | 1.8 | 19 | 8.5 | 1.3 | 21 | 8.2 | 1.8 | 21 |
| Discomfort | Exp 1 | 4 | 0 | 1 | 3 | 1 | 3 | 5 | 4.2 | 2 |
| | Exp 2 | 2.5 | 0.7 | 2 | 3.4 | 2.6 | 5 | 3.6 | 1 | 7 |
| Headache | Exp 1 | 3 | 0 | 1 | 4.3 | 2.5 | 3 | 5 | 1.4 | 2 |
| | Exp 2 | 2 | 0 | 1 | 4.6 | 1.8 | 5 | 5.3 | 3.5 | 3 |
| Blurry vision | Exp 1 | 5.5 | 3.5 | 2 | 3.5 | 1.4 | 2 | 4 | 0 | 1 |
| | Exp 2 | 1 | 0 | 1 | 5 | 0 | 1 | 0 | 0 | 0 |
| Skin itching | Exp 1 | 3 | 1.4 | 2 | 6 | 0 | 1 | 0 | 0 | 0 |
| | Exp 2 | 3 | 0 | 1 | 4 | 0 | 1 | 5 | 4.2 | 2 |
| Dizziness | Exp 1 | 0 | 0 | 0 | 0 | 0 | 0 | 4.5 | 0.7 | 2 |
| | Exp 2 | 7 | 0 | 1 | 3 | 0 | 2 | 6 | 0 | 1 |

In Experiment A, 10% of all participants (6/60) reported mild discomfort (3.8/10±2.2). When examined by groups, at least one participant per group reported discomfort. On the other hand, in Experiment B, 23% of participants (14/61) reported mild discomfort (3.4/10±1.4). Interestingly, the greatest number of participants reporting discomfort was in the sham group (n=7, score=3.6/10±1). When consulted specifically for side effects such as headaches, blurry vision, dizziness, and skin itching at the electrode site, similar results were found. In Experiment A, 15% or fewer participants in each group experienced a headache and 10% or fewer participants reported blurred vision. Only 10% of subjects in the sham group (but no other group) reported dizziness (n=2, score=4.5/10±0.7) and skin itching occurred in fewer than 10% of participants and only in the stimulation groups. On the other hand, in Experiment B, headaches were more commonly reported, with the 60 Hz group reporting five events, followed by the sham group with three, and the 120 Hz group with one. Only one participant in each active stimulation group reported blurred vision. Dizziness was reported in less than 10% of each group, similar to skin itching.

### 6. Discussion

This example explored the effects of single-session TNS on visuomotor adaptation in healthy adults. These experiments were initiated with the long-term goal of evaluating the feasibility of using this neuromodulatory technique as an adjuvant to conventional neurorehabilitation of sensorimotor dysfunction. TNS was delivered online and offline in separate experiments, using stimulation parameters derived from clinical applications. Evidence for both frequency- and timing-dependent effects of TNS on visuomotor adaptation was found. The results serve as a useful starting point for efforts aimed at developing optimal TNS parameters and protocols for augmenting conventional neurorehabilitation and enhancing human sensorimotor performance in industrial, athletic, military, and performing arts settings.

Experiment A assessed the effects of 60 and 120 Hz TNS on adaptation and found that neither frequencies enhance learning rates during the adaptation block. Quantification of the learning rates associated with visuomotor adaptation showed that TNS at 60 Hz slowed the learning, an effect that was more pronounced in the early adaptation phase. More specifically, both the fast and slow learning rate coefficients were significantly smaller than those in both the 120 Hz and sham groups, suggesting that TNS at 60 Hz had a detrimental effect on learning mechanisms. On the other hand, although the 120 Hz group showed some evidence of enhanced learning relative to sham, changes in directional error were modest and were not statistically significant.

Experiment B assessed the same 120 and 60 Hz frequencies with TNS applied during task performance (online). Even though learning curves associated with the active stimulation groups showed evidence of slightly faster adaptation, learning rates analyses showed no statistically significant differences between the active groups rates and those of the sham group. Interestingly, when results from this experiment were compared post hoc with those observed in Experiment A (offline TNS), significantly faster learning rates were observed for the online 60 Hz protocol compared to offline. This indicates that the effects of TNS on visuomotor adaptation depend not only on the frequency of stimulation but also on timing relative to task performance, i.e., online vs offline, at least for 60 Hz stimulation.

The Aston-Jones model of locus coeruleus-norepinephrine (LC-NE) system function may provide a possible explanation for the observed frequency-dependent effects. TNS effects are thought to be exerted via activation of the LC, with subsequent release of norepinephrine (NE) across the brain and resulting enhancement of synaptic plasticity. Similar to the Yerkes-Dodson law that relates performance with arousal, the Aston-Jones model relates changes in task performance resulting from neuromodulation to changes in LC activity using an inverted U-shaped curve. That is, poor performance is observed when either low or high tonic activity is present and more optimal performance occurs when moderate tonic and predominant phasic activity is present. In this scenario, the slower rates of learning observed with 60 Hz TNS relative to sham may have resulted from a shift in tonic LC firing, resulting in either very low or very high levels of tonic activity, both of which have been linked to poor performance. Along the same lines, learning rates for offline 120 Hz TNS, though not significantly different from sham rates in Experiment A, were still among the highest observed and were significantly different from those obtained with offline 60 Hz TNS. Moreover, fast learning rates resulting from 120 Hz offline TNS were also moderately correlated with reported attention levels. This suggests that higher frequencies of stimulation might lead to more optimal levels of LC firing and enhanced arousal and learning.

The observed timing-dependent effects may reflect a difference in the timescales of neural mechanisms that are believed to support endogenous neuromodulation. For example, online effects of transcranial stimulation have been attributed to the modulation of cortical excitability through the alteration of neuronal membrane properties by weak electric currents, a mechanism which acts on very short timescales. On the other hand, effects of transcutaneous cranial nerve stimulation are believed to occur via activation of brain stem nuclei, resulting in subsequent changes in cortical synaptic plasticity that happen on longer timescales. As a result, TNS modulation might be expected to be more amenable to offline rather than online stimulation, as it would allow more time for such changes to take hold. However, the opposite was observed here, at least with 60 Hz TNS, i.e., faster learning was observed with the online protocol compared to the offline protocol. It is possible then that TNS effects are mediated by both transcranial and transcutaneous mechanisms. Transcranial mechanisms might be invoked due to the location of the electrodes, which would allow some current to reach frontal cortical areas, a scenario supported by current flow models. To determine whether transcranial, transcutaneous or both mechanisms underlie TNS effects, future studies might consider blocking the trigeminal nerve using skin anesthetics to eliminate or reduce contributions from peripheral cutaneous receptors. This general approach was recently used to probe the source of behavioral effects when tDCS was applied to the posterior cervical region. Residual behavioral effects of stimulation were observed even after blocking the occipital nerve, suggesting that some effects of stimulation in this region result via transcranial mechanisms, which could be same for TNS.

a. Sham Groups Performance

In both experiments, a subset of participants in the sham group (24% in Experiment A and 38% in Experiment B) reported perceiving the stimulation even though no current was applied to them. Interestingly, these participants learned the rotation at a rate similar to the best performers of each experiment. Thus, in Experiment 1, the sham-felt subgroup tracked the learning behavior of the 120 Hz group while in Experiment 2, the sham-felt tracked the 120 and 60 Hz groups early during the adaptation and then diverged in the late stage, ending up with higher directional error. On the other hand, participants who did not report perceiving stimulation (sham-no) generally demonstrated the slowest learning rates. Placebo effects are thought to result from a combination of factors such as the environment, expectations about the treatment, as well as physiological effects. Environmental factors appear to play a key role in transcranial neuromodulation studies, where strong placebo effects have been reported. The presence of equipment sounds generated by this equipment, the placement of electrodes on the head, and interactions with the experimenter, among other factors, can potentially provide cues to participants about the treatment, contributing to placebo effects. In the tested protocol, subjects in the sham groups used the same electrode placement as the active groups, the stimulator made the same clicking sound every time it was supposed to be active, and the experimenter mimicked the same protocol used for the active groups to look for submaximal tolerable thresholds, but no current was delivered. Nevertheless, some participants still performed in a manner suggestive of a placebo effect. Furthermore, the expectation of receiving a treatment, i.e., stimulation or a drug, has also been shown to be a relevant factor in placebo/sham-controlled studies and may have contributed to the apparent placebo effects observed here. Although participants' expectations were not probed in the survey, those who perceived the stimulation might have had a different expectation from those who did not perceive it. That is, not feeling the stimulation might have lowered expectations in these participants, potentially affecting their performance. In contrast, feeling the stimulation might have affected the performance of those participants positively.

b. Side Effects

Ten percent (10%) of participants in Experiment A reported experiencing some discomfort during the experiment, while 23% reported discomfort in Experiment B. This is surprising given that participants were instructed to select a comfortable intensity and were told that they could ask to reduce or stop the stimulation at any time. Additionally, subjects in the sham group also reported experiencing discomfort, even though they did not receive any current. Importantly, our questionnaire did not probe the source of the reported discomfort, thus it is possible that this perception was unrelated to the stimulation. For instance, several participants across all groups complained about arm fatigue, which could have been source of this discomfort due to the highly repetitive nature of the reaching task that was used.

Participants were also asked specifically about whether they experienced headaches, blurry vision, skin itching, and dizziness in both experiments. In Experiment A, headaches were reported by the largest number of participants, with five (23.8%) in the 60 Hz group, followed by three (14.3%) in the sham group and only one in the 120 Hz group (5.3%). These findings align with the results of Experiment A, where three (14.3%) instances were reported in the 60 Hz group, two (10.5%) in the sham group and one in the 120 Hz group (5%). Although headaches and skin itching have been associated with TNS in other studies, in these experiments it is difficult to attribute them exclusively to the stimulation because subjects in the sham group also reported experiencing them, even though no current was applied. This suggests that some of these headaches may have resulted from experimental factors other than the stimulation. For example, prolonged exposure to VR environments is known to contribute to cybersickness, which manifests as headaches and eyestrain, among other side effects. Given the relatively long duration of our experiments (2 hours), it is possible that our VR environment, and not the TNS, was the root cause of some or most reports of headache. In support of this idea, it is noteworthy that one participant discontinued the experiment due to eyestrain.

c. Stimulation Parameters and Protocol

In these experiments, several stimulation parameters were tested, which to our judgment, provided reasonable starting points to assess the feasibility of the effects of TNS on motor learning. However, these parameters are clearly a minimal sample of the overall stimulation parameter space. For example, the effects of a single session of approximately 10 min of effective stimulation, which, as described above, produced modest effects. However, in previous studies, therapeutic effects of TNS have been shown to require weeks or months of treatment. Therefore, increasing the session length and/or holding multiple sessions across several days might enhance the effects of TNS on motor learning. Moreover, cycled stimulation (30 s ON/OFF) was utilized because it has been commonly used clinically to address the symptoms of ADHD and DRE. However, continuous TNS has been shown to have beneficial effects on migraine, which warrants further investigation into this mode of application in studies of TNS efficacy. Finally, future work should consider pairing the stimulation with movements, which have shown to be effective for rehabilitation using VNS.

In both experiments, the stimulation intensity was determined by each participant's self-selected submaximal tolerable threshold, which offered participants more control over the dose they received. Although mean currents did not differ significantly across groups in either experiment, current levels within each group were highly variable, which might have contributed to the modest differences among groups reported here. Given that learning rates within each group were also somewhat variable within each group, potential associations between learning rates and intensities were explored. Statistically significant correlations were only found for the 60 Hz group in Experiment A. An alternative approach would have involved using a fixed current for all subjects, similar to the approach used in tDCS studies, where a fixed intensity of 2 mA is typically employed.

d. Behavioral Task

Here, a visuomotor rotation task was used to study motor adaptation, a form of motor learning where previously learned movements are adapted following a transient perturbation in the environment. This task was chosen because it has been extensively studied both as a means to understand motor learning in general and also as a means to quantify the effects of neuromodulation on motor learning. Despite the evidence provided here suggesting that TNS affects motor learning behavior, the magnitudes of these effects were modest. Results obtained using offline 120 Hz stimulation, which trended toward faster learning rates, but which were not statistically different from sham, may represent a floor effect on visuomotor learning in healthy young adults. However, recent studies using older adults suggest that the degree of visuomotor adaptation is reduced and more variable in this population. Thus, an older population may gain greater visuomotor learning benefits.

What is claimed:

1. A method of improving motor learning in a subject comprising delivering an electrical current to the subject's trigeminal nerves.

2. The method of claim 1, wherein the electrical current to the subject's trigeminal nerves is 2.0-4.5 mA.

3. The method of claim 1, wherein the electrical current delivered to the subject's trigeminal nerves has a frequency of 50-150 Hz or about 3 kHz.

4. The method of claim 1, wherein the electrical current is delivered as a biphasic symmetric square waveform in 30 s intervals.

5. The method of claim 4, wherein the electrical current is delivered a pulse width 250 μs and an interphase interval is 1 μs.

6. The method of claim 4, wherein the electrical current is delivered to the left and right supraorbital branches of the subject's trigeminal nerve.

7. The method of claim 1, wherein the subject's visuomotor adaptation is improved.

8. The method of claim 1, wherein the subject does not have a brain injury or has not been diagnosed with a brain disease or disorder.

9. The method of claim 8, wherein the subject is a healthy subject.

10. The method of claim 1, wherein the subject is recovering from a stroke.

11. A method of improving motor learning in a subject comprising delivering an electrical current to the subject's occipital nerves.

12. The method of claim 11, wherein the electrical current to the subject's trigeminal nerves is 9.5-17 mA.

13. The method of claim 11, wherein the electrical current delivered to the subject's trigeminal nerves has a frequency of 1-5 Hz or about 3 kHz.

14. The method of claim 11, wherein the electrical current is delivered as a biphasic symmetric square waveform in 30 s intervals.

15. The method of claim 12, wherein the electrical current is delivered a pulse width 250 μs and an interphase interval is 1 μs.

16. The method of claim 15, wherein the electrical current is delivered to target the subject's C2/C3 dermatomes.

17. The method of claim 11, wherein the subject's visuomotor adaptation is improved.

18. The method of claim 11, wherein the subject does not have a brain injury or has not been diagnosed with a brain disease or disorder.

19. The method of claim 18, wherein the subject is a healthy subject.

20. The method of claim 11, wherein the subject is recovering from a stroke.
